(19) United States
(12) Patent Application Publication
(10) Pub. No.: US 2018/0256673 A1
(43) Pub. Date: Sep. 13, 2018

(54) METHODS FOR TREATING FUNGAL INFECTIONS

(71) Applicant: Cidara Therapeutics, Inc., San Diego, CA (US)

(72) Inventors: James Michael BALKOVEC, Martinsville, NJ (US); Kenneth BARTIZAL, La Jolla, CA (US); Jeffrey Brian LOCKE, La Jolla, CA (US); Voon ONG, San Diego, CA (US); Taylor SANDISON, Encinitas, CA (US); Dirk THYE, San Diego, CA (US); David S. PERLIN, New York, NY (US); Kenneth Duke JAMES, Jr., Mebane, NC (US)

(21) Appl. No.: 15/760,029

(22) PCT Filed: Sep. 16, 2016
(86) PCT No.: PCT/US2016/052159

§ 371 Date: Mar. 14, 2018

Related U.S. Application Data
(60) Provisional application No. 62/219,628, filed on Sep. 16, 2015.
(60) Provisional application No. 62/309,325, filed on Mar. 16, 2016.
(60) Provisional application No. 62/326,492, filed on Apr. 22, 2016.
(60) Provisional application No. 62/350,579, filed on Jun. 15, 2016.

(51) Int. Cl. A61K 38/12; A61P 31/10; A61K 9/00

(52) U.S. Cl. CPC A61K 38/12; A61K 9/0019; A61P 31/10

(57) ABSTRACT

The disclosure relates to methods of treating a fungal infection in a subject by administering to the subject a salt of Compound 1, or a neutral form thereof. The methods of the disclosure can be useful in subjects suffering from treatment resistant infections, useful in subjects who have previously failed treatment with an anti-fungal therapy, and useful for suppressing the emergence of resistant strains in infected subjects.

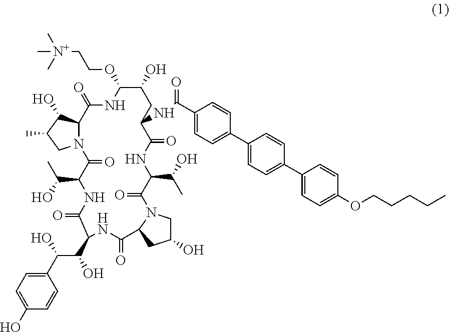 [SMILES: CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1]

### BACKGROUND

Echinocandins are members of a leading class of antifungal agents for the treatment of fungal infections. These compounds target the cell wall by preventing the production of 1,3-β-D-glucan through inhibition of the catalytic subunit of 1,3-β-D-glucan synthase enzyme complex. The three echinocandins approved by the Food and Drug Administration (FDA) for the treatment of invasive fungal infections (caspofungin, anidulafungin, and micafungin) are available only in intravenous formulations. The daily administration of these antifungal agents may contribute to the rise in reports of breakthrough fungal infections, e.g., Candida infections. There is a need in the art for improved methods of treatment for fungal infections.

### SUMMARY

The disclosure relates to methods of treating a drug-resistant fungal infection in a subject (e.g., a human) by administering to the subject a salt of Compound 1, or a neutral form thereof. In some embodiments, the subject and/or the fungal infection has failed treatment with an echinocandin therapy, a polyene therapy, flucytosine therapy, and/or an azole therapy. A salt of Compound 1, or a neutral form thereof, displays long-acting pharmacokinetics with a long half-life and slow clearance and strong activities against fungi (e.g., Candida, Aspergillus) having either wild-type or mutant 1,3-β-D-glucan synthase enzyme complex.

In a first aspect, the disclosure features a method of treating a drug-resistant fungal infection in a subject (e.g., a human) including administering a salt of Compound 1,

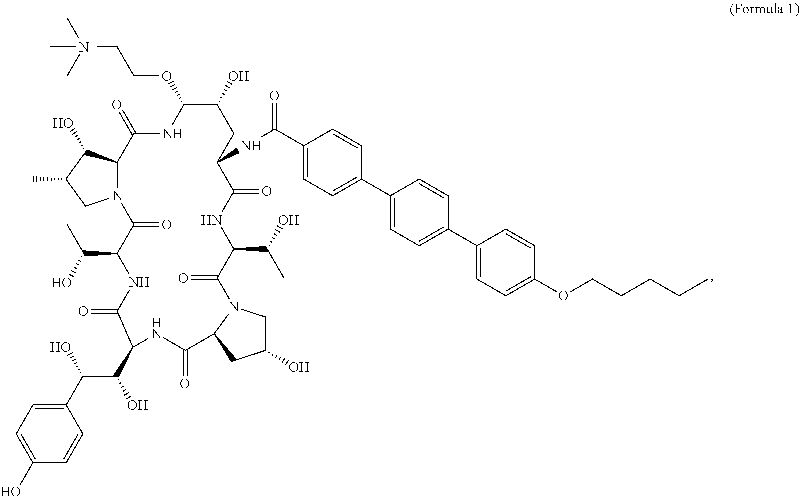 [SMILES: CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1]

or a neutral form thereof, to the subject in an amount and for a duration sufficient to treat the drug-resistant fungal infection.

In another aspect, the disclosure features a method of treating a fungal infection in a subject (e.g., a human) who has failed treatment with an antifungal therapy. The method includes administering a salt of Compound 1, or a neutral form thereof, to the subject in amounts and for a duration sufficient to treat the fungal infection.

In some embodiments of either of the above aspects, the fungal infection is caused by a fungus having a mutant 1,3-β-D-glucan synthase enzyme complex.

In another aspect, the disclosure features a method of treating a fungal infection in a subject including administering a salt of Compound 1, or a neutral form thereof, to the subject, in amounts and for a duration sufficient to treat the fungal infection, wherein the fungal infection is caused by a fungus having a mutant 1,3-β-D-glucan synthase enzyme complex. In some embodiments of any of the above aspects, the method includes administering two or more doses of a salt of Compound 1, or a neutral form thereof, to the subject in amounts and for a duration sufficient to treat the drug-resistant fungal infection.

In some embodiments of any of the above aspects, the administering step includes intravenously administering doses of about 150 mg to about 800 mg of a salt of Compound 1, or a neutral form thereof, (e.g., 200±50, 300±50, 400±50, 500±50, 600±50, 700±50, or 750±50 mg) to the subject. In some embodiments, two or more doses (e.g., 2 to 7, 2 to 3, 3 to 4, or 5 to 7 doses) are administered to the subject over a period of 1 to 4 weeks (e.g., over a period of less than 2 weeks, 3 weeks, or 4 weeks).

In another aspect, the disclosure features a method of treating a fungal infection in a subject (e.g., a human) including intravenously administering doses of about 550 mg to about 800 mg of a salt of Compound 1, or a neutral form thereof, (e.g., 600±50, 650±50, 700±50, or 750±50 mg) to the subject, wherein two or more doses (e.g., 2 to 7, 2 to 3, 3 to 4, or 5 to 7 doses) are administered to the subject over a period of 1 to 4 weeks.

In another aspect, the disclosure features a method of treating a fungal infection in a subject (e.g., a human) including intravenously administering doses of about 150 mg to about 800 mg of a salt of Compound 1, or a neutral form thereof, (e.g., 250±50, 300±50, 400±50, 500±50, 600±50, 700±50, or 750±50 mg) one to three times per week to the subject for 2 to 4 weeks (e.g., over a period of less than 2 weeks, 3 weeks, or 4 weeks).

In another aspect, the disclosure features a method of treating a fungal infection in a subject (e.g., a human) including intravenously administering two or more doses of a composition including about 150 mg to about 800 mg of a salt of Compound 1, or a neutral form thereof, (e.g., 250±50, 300±50, 400±50, 500±50, 600±50, 700±50, or 750±50 mg) to the subject.

In some embodiments, the administered amount maintains at least a mutant prevention concentration of Compound 1 in plasma for a period of at least 8 hours, 12 hours, 16 hours, 24 hours, 2 days, 4 days, or 1 week.

In some methods, Compound 1 in salt or neutral form is intravenously administered to a subject in two or more weekly doses (e.g., 2, 3, 4, 5, 6, 7, 8, 9, 10, 11, or 12 doses), wherein the first dose contains about 400 mg of Compound 1 in salt or neutral form and each of the subsequent doses contains about 200 mg of Compound 1 in salt or neutral form. In some embodiments, the first dose includes about 400 mg of Compound 1 in salt or neutral form, and each of the remaining doses includes about 200 mg of Compound 1 in salt or neutral form. In some embodiments, the dosing regimen consists of (a) intravenously administering a first dose of about 400 mg of Compound 1 in salt or neutral form, (b) intravenously administering a second dose of about 200 mg of Compound 1 in salt or neutral form, and (c) optionally intravenously administering a third dose of about 200 mg of Compound 1 in salt or neutral form, wherein the first dose is administered on day 1, the second dose is administered on day 8, and the third dose, if administered, is administered on day 15.

In some methods, Compound 1 in salt or neutral form is intravenously administered to a subject in two or more weekly doses (e.g., 2, 3, 4, 5, 6, 7, 8, 9, 10, 11, or 12 doses) of about 400 mg of Compound 1 in salt or neutral form. In some embodiments, the dosing regimen consists of (a) intravenously administering a first dose of about 400 mg of Compound 1 in salt or neutral form, (b) intravenously administering a second dose of about 400 mg of Compound 1 in salt or neutral form, and (c) optionally intravenously administering a third dose of about 400 mg of Compound 1 in salt or neutral form, wherein the first dose is administered on day 1, the second dose is administered on day 8, and the third dose, if administered, is administered on day 15.

As used herein, the amount in each dose refers to the amount of Compound 1 (structure of Formula I shown above) that does not include the negative counterion (e.g., an acetate) if Compound 1 is in its salt form. For example, a dose of about 400 mg or 200 mg of Compound 1 in salt or neutral form refers to 400 mg or 200 mg of Compound 1, not including the acetate ion if Compound 1 is in an acetate salt form.

In some embodiments, the third dose of about 200 mg of Compound 1 in salt or neutral form is administered if on day 15 mycological eradication and/or clinical cure is not achieved in the subject. In some embodiments, the third dose of about 400 mg of Compound 1 in salt or neutral form is administered if on day 15 mycological eradication and/or clinical cure is not achieved in the subject. In some embodiments, the mycological eradication is determined by two negative blood cultures drawn at 12 hours apart without intervening positive blood cultures.

In some embodiments, the third dose of about 200 mg of Compound 1 in salt or neutral form is administered if on day 15 the subject displays symptoms of a fungal infection. In some embodiments, the third dose of about 400 mg of Compound 1 in salt or neutral form is administered if on day 15 the subject displays symptoms of a fungal infection. In some embodiments, symptoms of the fungal infection includes fever, cough, shortness of breath, weight loss, and/or night sweats.

In some embodiments of the methods described herein, Compound 1 in salt or neutral form is administered for 2-12 doses (e.g., 2-3 doses). In some embodiments, Compound 1 in salt or neutral form is administered until mycological eradication and/or clinical cure is achieved as determined by a standard test known in the art. In some embodiments, mycological eradication is defined as two negative blood cultures drawn at 12 hours apart without intervening positive blood cultures and no change of antifungal therapy for the fungal infection. In some embodiments, Compound 1 in salt or neutral form is administered until the subject is free of symptoms of the fungal infection, such as fever, cough, shortness of breath, weight loss, and night sweats, as determined by a physician.

In some embodiments of any of the above aspects described herein, the fungal infection is an echinocandin-resistant, polyene-resistant, flucytosine-resistant, or azole-resistant fungal infection. In some embodiments, the fungal infection is an echinocandin-resistant infection. In some embodiments, the subject has failed treatment with an echinocandin therapy. In some embodiments, the subject has failed treatment with anidulafungin, micafungin, or caspofungin. In some embodiments, the subject has failed treatment with a polyene therapy, flucytosine therapy, or an azole therapy. In some embodiments, the subject has failed treatment with another antifungal drug and/or 1,3-β-D-glucan synthase inhibitor, such as enfumafungin and SCY-078.

In some embodiments of any of the above aspects described herein, the fungal infection is caused by a fungus having a mutant 1,3-β-D-glucan synthase enzyme complex including one or more mutations in FKS genes.

In some embodiments of any of the above aspects described herein, the fungal infection is a Candida infection. In some embodiments, the Candida is selected from the group consisting of Candida albicans, C. glabrata, C. dubliniensis, C. krusei, C. parapsilosis, C. tropicalis, C. orthopsilosis, C. guilliermondii, C. rugosa, and C. lusitaniae. In some embodiments, the Candida is Candida albicans. In some embodiments, the Candida is Candida glabrata.

In some embodiments of any of the above aspects described herein, the Candida infection is candidemia, oropharyngeal candidiasis, esophageal candidiasis, mucosal candidiasis, genital candidiasis, vulvovaginal candidiasis, gastrointestinal candidiasis, rectal candidiasis, hepatic candidiasis, renal candidiasis, pulmonary candidiasis, splenic candidiasis, cardiovascular candidiasis (e.g., endocarditis), or invasive candidiasis.

In some embodiments of any of the above aspects described herein, the fungal infection is an Aspergillus infection or a dermatophyte infection.

In some embodiments of any of the above aspects described herein, the administering step includes administering a salt of Compound 1, or a neutral form thereof, topically, intravaginally, intraorally, intravenously, intramuscularly, intradermally, intraarterially, subcutaneously, orally, or by inhalation.

In some embodiments of any of the above aspects described herein, the administering step includes administering a salt of Compound 1, or a neutral form thereof, intravenously. As used herein, the terms “intravenous administration” or “intravenously administering” refer to intravenous bolus injection or infusion of a drug to a subject.

In another aspect, the disclosure features a method of killing an echinocandin-resistant, polyene-resistant, flucytosine-resistant, or azole-resistant Candida including exposing the echinocandin-resistant, polyene-resistant, flucytosine-resistant, or azole-resistant Candida to a salt of Compound 1, or a neutral form thereof, in an amount and for a duration sufficient to kill the echinocandin-resistant, polyene-resistant, flucytosine-resistant, or azole-resistant Candida. In some embodiments, the Candida is selected from the group consisting of Candida albicans, C. glabrata, C. dubliniensis, C. krusei, C. parapsilosis, C. tropicalis, C. orthopsilosis, C. guilliermondii, C. rugosa, and C. lusitaniae. In some embodiments, the Candida is Candida albicans. In some embodiments, the Candida is Candida glabrata.

In another aspect, the disclosure features a method of treating an echinocandin-resistant fungal infection in a subject (e.g., a human) by intravenously administering a salt of Compound 1, or a neutral form thereof, to the subject in an amount sufficient to treat the echinocandin-resistant fungal infection, wherein the echinocandin-resistant fungal infection is caused by a fungus having a mutant 1,3-β-D-glucan synthase enzyme complex including one or more mutations in FKS genes.

In another aspect, the disclosure features a method of treating an echinocandin-resistant fungal infection in a subject (e.g., a human) by intravenously administering two or more doses of a salt of Compound 1, or a neutral form thereof, to the subject in an amount sufficient to treat the echinocandin-resistant fungal infection over a period of 1 to 4 weeks, wherein the echinocandin-resistant fungal infection is caused by a fungus having a mutant 1,3-β-D-glucan synthase enzyme complex including one or more mutations in FKS genes.

In another aspect, the disclosure features a method of treating a fungal infection in a subject (e.g., a human) by intravenously administering Compound 1 in salt or neutral form, to the subject in a dosing regimen that maintains at least a mutant prevention concentration of Compound 1 in the plasma of the subject for a period of at least 8 hours, 12 hours, 16 hours, 24 hours, 2 days, 4 days, or 1 week. In another aspect, the disclosure features a method of treating a fungal infection in a subject (e.g., a human) by intravenously administering two or more doses of about 150 mg to about 800 mg of Compound 1 in salt or neutral form, to the subject in a dosing regimen that maintains at least a mutant prevention concentration of Compound 1 in plasma for a period of at least 8 hours, 12 hours, 16 hours, 24 hours, 2 days, 4 days, or 1 week. In particular embodiments, doses of 200±50, 300±50, 400±50, 500±50, 600±50, 700±50, or 750±50 mg are administered to the subject in two or more times (e.g., 2 to 7, 2 to 3, 3 to 4, or 5 to 7 doses) over a period of 1 to 4 weeks (e.g., over a period of less than 2 weeks, or 3 weeks, or 4 weeks).

In another aspect, the disclosure features a method of administering Compound 1 to a subject (e.g., a human), wherein the method consisting of (a) intravenously administering a first dose including 400 mg of Compound 1 in salt or neutral form, (b) intravenously administering a second dose including 200 mg of Compound 1 in salt or neutral form, and (c) optionally intravenously administering a third dose including 200 mg of Compound 1 in salt or neutral form, wherein the first dose is administered on day 1, the second dose is administered on day 8, and the third dose, if administered, is administered on day 15.

In another aspect, the disclosure features a method of administering Compound 1 to a subject (e.g., a human), wherein the method consisting of (a) intravenously administering a first dose including 400 mg of Compound 1 in salt or neutral form, (b) intravenously administering a second dose including 400 mg of Compound 1 in salt or neutral form, and (c) optionally intravenously administering a third dose including 400 mg of Compound 1 in salt or neutral form, wherein the first dose is administered on day 1, the second dose is administered on day 8, and the third dose, if administered, is administered on day 15. In some embodiments of the aspects described herein, Compound 1 is administered over a time period of 30 to 180 minutes (e.g., over 30±5 minutes, 60±5 minutes, 90±5 minutes, 120±5 minutes, 150±5 minutes, or 180±5 minutes).

In some embodiments, Compound 1 is administered as an aqueous pharmaceutical composition. In some embodiments, the pharmaceutical composition has a pH of from 4.0 to 8. In some embodiments, Compound 1 salt is the acetate salt of Compound 1.

### Definitions

As used herein, the term “a salt of Compound 1” refers to a salt of the compound of Formula 1. Compound 1 has a structure (below) in which the tertiary ammonium ion positive charge of Compound 1 is balanced with a negative counterion (e.g., an acetate) in its salt form.

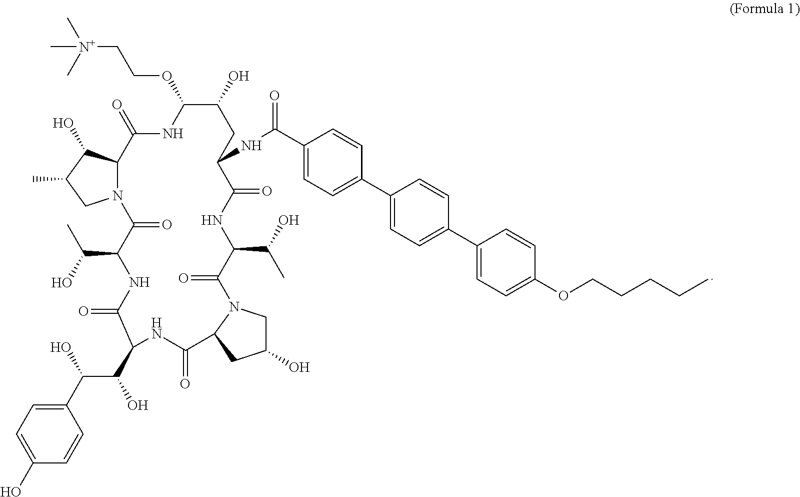 [SMILES: CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1]

As used herein, the term “a neutral form” includes to zwitterionic forms of Compound 1 in which the compound of Formula 1 has no net positive or negative charge. The zwitterion is present in a higher proportion in basic medium (e.g., pH 9) relative to Compound 1 or a salt of Compound 1. In some embodiments, the zwitterion may also be present in its salt form.

As used herein, the term “a drug-resistant fungal infection” refers to a fungal infection that is refractory to treatment with a drug, e.g., an antifungal drug. In such infections the fungus that causes the infection is resistant to treatment with one or more antifungal drugs (e.g., an antifungal drug-resistant strain of fungus (e.g., an antifungal drug-resistant strain of Candida spp.)). Antifungal drugs include, but are not limited to, echinocandins, polyene compounds, flucytosine, and azole compounds. Fungal infections may be caused by a fungus in the genus, e.g., Candida (e.g., Candida albicans, Candida glabrata) or Aspergillus (e.g., Aspergillus fumigatus). In some embodiments, a fungal infection may also be a dermatophyte infection.

As used herein, the term “Candida infection” refers to an infection caused by a fungus in the genus Candida. Examples of fungi in the genus Candida include, but are not limited to, Candida albicans, C. glabrata, C. dubliniensis, C. krusei, C. parapsilosis, C. tropicalis, C. orthopsilosis, C. guilliermondii, C. rugosa, and C. lusitaniae. Examples of Candida infections include, but are not limited to, candidemia, oropharyngeal candidiasis, esophageal candidiasis, mucosal candidiasis, genital candidiasis, vulvovaginal candidiasis, gastrointestinal candidiasis, rectal candidiasis, hepatic candidiasis, renal candidiasis, pulmonary candidiasis, splenic candidiasis, cardiovascular candidiasis (e.g., endocarditis), and invasive candidiasis.

As used herein, the term “Aspergillus infection” refers to an infection caused by a fungus in the genus Aspergillus. Examples of fungi in the genus Aspergillus include, but are not limited to, Aspergillus fumigatus, A. flavus, A. terreus. A. niger, A. candidus, A. clavatus, and A. ochraceus. Examples of Aspergillus infections include, but are not limited to, aspergillosis (e.g., invasive aspergillosis, central nervous system aspergillosis, or pulmonary aspergillosis).

As used herein, the term “dermatophyte infection” refers to an infection caused by dermatophytes, which are fungi that require keratin for growth. Dermatophytes are fungi in the genus Microsporum, Epidermophyton, and Trichophyton. These fungi can cause superficial infections of the skin, hair, and/or nails. Dermatophytes are spread by direct contact from other people (anthropophilic organisms), animals (zoophilic organisms), and soil (geophilic organisms), as well as indirectly from fomites.

As used herein, the term “echinocandin-resistant fungal infection” refers to a fungal infection that is refractory to treatment with an echinocandin. In such infections the fungus that causes the infection is resistant to treatment with one or more echinocandins. The one or more echinocandins are cyclic lipopeptides that inhibit the synthesis of glucan in the cell wall by inhibition of the 1,3-β-D-glucan synthase enzyme complex. The one or more echinocandins referred to in the term “echinocandin-resistant fungal infection” include micafungin, caspofungin, and anidulafungin, but does not include a salt of Compound 1, or a neutral form thereof. Thus, using the methods of the disclosure, a salt of Compound 1, or a neutral form thereof, can be used to treat micafungin-resistant, caspofungin-resistant, and/or anidulafungin-resistant fungal infections.

As used herein, the term “polyene-resistant fungal infection” refers to a fungal infection that is refractory to treatment with a polyene compound. In such infections, the fungus that causes the infection is resistant to treatment with one or more polyene compounds. Polyene compounds are compounds that insert into fungal membranes, bind to ergosterol and structurally related sterols in the fungal membrane, and disrupt membrane structure integrity, thus causing leakage of cellular components from a fungus that causes infection. Polyene compounds typically include large lactone rings with three to eight conjugated carbon-carbon double bonds and may also contain a sugar moiety and an aromatic moiety. Examples of polyene compounds include, but are not limited to, 67-121-A, 67-121-C, amphotericin B, arenomvcin B, aurenin, aureofungin A, aureotuscin, candidin, chinin, demethoxyrapamycin, dermostatin A, dermostatin B, DJ-400-B₁, DJ-400-B₂, elizabethin, eurocidin A, eurocidin B, filipin I, filipin II, filipin III, filipin IV, fungichromin, gannibamycin, hamycin, levorin A₂, lienomycin, lucensomycin, mycoheptin, mycoticin A, mycoticin B, natamycin, nystatin A, nystatin A₃, partricin A, partricin B, perimycin A, pimaricin, polifungin B, rapamycin, rectilavendomvcin, rimocidin, roflamycoin, tetramycin A, tetramycin B, tetrin A, and tetrin B.

As used herein, the term “flucytosine-resistant fungal infection” refers to a fungal infection that is refractory to treatment with the synthetic antifungal drug flucytosine. A brand name for flucytosine is Ancobon®.

As used herein, the term “azole-resistant fungal infection” refers to a fungal infection that is refractory to treatment with an azole compound. In such infections the fungus that causes the infection is resistant to treatment with one or more azole compounds. The azole compounds referred to in the term “azole-resistant fungal infection” are antifungal compounds that contain an azole group, which is a five-membered heterocyclic ring having at least one N and one or more heteroatoms selected from N, O, or S. Antifungal azole compounds function by binding to the enzyme 14α-demethylase and disrupt, inhibit, and/or prevent its natural function. The enzyme 14α-demethylase is a cytochrome P450 enzyme that catalyzes the removal of the C-14 α-methyl group from lanosterol before lanosterol is converted to ergosterol, an essential component in the fungal cell wall. Therefore, by inhibiting 14α-demethylase, the synthesis of ergosterol is inhibited. Examples of azole compounds include, but are not limited to, VT-1161, VT-1598, fluconazole, albaconazole, bifonazole, butoconazole, clotrimazole, econazole, efinaconazole, fenticonazole, isavuconazole, isoconazole, itraconazole, ketoconazole, luliconazole, miconazole, omoconazole, oxiconazole, posaconazole, pramiconazole, ravuconazole, sertaconazole, sulconazole, terconazole, tioconazole, and voriconazole.

As used herein, the term “antifungal therapy” refers to treatment of a fungal infection using an antifungal drug. Antifungal drugs used in an antifungal therapy include, but are not limited to, echinocandins, polyene compounds, flucytosine, azole compounds, enfumafungin, and SCY-078. As described herein, an aspect of the disclosure is a method of treating a fungal infection in a subject who has failed treatment with an antifungal therapy. The antifungal drugs used in the antifungal therapy in this aspect of the disclosure do not include a salt of Compound 1, or a neutral form thereof. As used herein, the term “echinocandin therapy” refers to a treatment for a fungal infection using an echinocandin (such as micafungin, caspofungin, and anidulafungin, but not a salt of Compound 1, or a neutral form thereof). As described herein, in some embodiments, a subject who has failed treatment with an echinocandin therapy may be administered a salt of Compound 1, or a neutral form thereof, to treat a fungal infection. In some embodiments, a subject having a fungal infection may be administered a salt of Compound 1, or a neutral form thereof, if the fungal infection has failed treatment with an echinocandin therapy.

As used herein, the term “polyene therapy” refers to a treatment for a fungal infection using a polyene compound. As described herein, in some embodiments, a subject who has failed treatment with a polyene therapy may be administered a salt of Compound 1, or a neutral form thereof, to treat a fungal infection. In some embodiments, a subject having a fungal infection may be administered a salt of Compound 1, or a neutral form thereof, if the fungal infection has failed treatment with a polyene therapy.

As used herein, the term “azole therapy” refers to a treatment for a fungal infection using an azole compound. Examples of antifungal azole compounds include, but are not limited to, VT-1161, VT-1598, fluconazole, albaconazole, bifonazole, butoconazole, clotrimazole, econazole, efinaconazole, fenticonazole, isavuconazole, isoconazole, itraconazole, ketoconazole, luliconazole, miconazole, omoconazole, oxiconazole, posaconazole, pramiconazole, ravuconazole, sertaconazole, sulconazole, terconazole, tioconazole, and voriconazole. As described herein, in some embodiments, a subject who has failed treatment with an azole therapy may be administered a salt of Compound 1, or a neutral form thereof, to treat the fungal infection. In some embodiments, a subject having a fungal infection may be administered a salt of Compound 1, or a neutral form thereof, if the fungal infection has failed treatment with an azole therapy.

As used herein, the term “1,3-β-D-glucan synthase enzyme complex” refers to the multi-subunit enzyme complex responsible for the synthesis of f 1,3-β-D-glucan, which is an essential component in the fungal cell wall. A mutant 1,3-β-D-glucan synthase enzyme complex refers to a 1,3-β-D-glucan synthase enzyme complex having one or more mutations in one or more subunits of the enzyme complex. In some embodiments, the one or more mutations are in the FKS genes (FKS1, FKS2, FKS3), which encode the catalytic subunit of 1,3-β-D-glucan synthase enzyme complex.

As used herein, the term “about” refers to a range of values that is ±10% of specific value. For example, “about 150 mg” includes ±10% of 150 mg, or from 135 mg to 165 mg. Such a range performs the desired function or achieves the desired result. For example, “about” may refer to an amount that is within less than 10% of, within less than 5% of, within less than 1% of, within less than 0.1% of, and within less than 0.01% of the stated amount.

By “dose” is meant the amount of Compound 1 administered to the subject (e.g., a human). By “clinical cure” is meant complete resolution of most or all of the clinical signs and symptoms of candidemia which were present at baseline and no new signs/symptoms or complications attributable to candidemia.

As used herein, the term “spontaneous mutations” refers to mutations that arise naturally and not as a result of the intentional used of certain mutagens. Spontaneous mutations may arise from a variety of sources, e.g., errors in DNA replication, spontaneous lesions, and transposable genetic elements. In some embodiments, the frequencies of spontaneous mutations may be calculated by dividing the number of resistant colonies on a given plate by the starting inoculum plated. In some embodiments, spontaneous mutations may confer drug resistance in a fungus (e.g., a fungus in the genus Candida or Aspergillus, or a dermatophyte). For example, a fungus may be initially susceptible to an antifungal drug, however, as the fungus grows and replicates over a period of time, spontaneous mutations may occur in the fungus that confer resistance in the fungus against the antifungal drug. As described herein, an infection may an echinocandin-resistant, a polyene-resistant, a flucytosine-resistant, or an azole-resistant fungal infection if the fungus causing the infection develops spontaneous mutations that allow the fungus to be resistant to one or more antifungal drugs (e.g., echinocandins, polyene compounds, flucytosine, and/or azole compounds). In some embodiments, a fungus may develop spontaneous mutations in the presence or absence of an antifungal drug.

As used herein, the term “mutant prevention concentration” or “MPC” refers to the concentration of a drug sufficient to suppress the development of all but very rare spontaneous mutants. The range of drug concentrations between the MIC and MPC represents a mutant selection window wherein de novo mutants are most likely to occur. Therapeutic regimens that maximize the duration of drug concentrations in excess of the MPC thereby minimize the potential for resistance development during the course of therapy.

Selection of resistance strains in vivo predominantly occurs over a range of drug concentrations falling between the MIC and MPC values. Dosing paradigms that result in plasma drug concentrations in excess of the MPC are therefore more desirable and effective in preventing the development of de novo mutants during the course of therapy (see, e.g., Drlica et al., J. Antimicrob. Chemother. 52:11-17, 2003). Currently approved treatment regimens for caspofungin, micafungin, and anidulafungin involve once-daily dosing at levels such that the Cₘₐₓ is unlikely to be equivalent to or exceed the MPC at any point during treatment. The MPC determined for Compound 1 vs. C. albicans and C. glabrata was 16 μg/ml (see Example 2). Modeling of Compound 1 total plasma concentrations based on in vivo pharmacokinetic data allows us to calculate that an IV administration of Compound 150 mg would be sufficient to generate concentrations in excess of 16 μg/ml. For other strains and/or fungal species, the dosing regimen the produces a plasma mutant prevention concentration can be one in which the plasma concentration is in excess of 20 μg/ml, 24 μg/ml, 30 μg/ml, or 36 μg/ml. Compound 1 has the potential to be dosed at levels exceeding the MPC, and thus have a stronger mutant prevention capacity than existing approved echinocandin treatment regimens.

All publications, patents, and patent applications are herein incorporated by reference in their entirety to the same extent as if each individual publication, patent or patent application was specifically and individually indicated to be incorporated by reference in its entirety.

### DESCRIPTION OF THE DRAWINGS

FIG. 1A shows effects of Compound 1 (CMP1) and micafungin (MCF) on kidney burdens in mice infected with C. albicans wild-type strain ATCC 90028.

FIG. 1B shows effects of CMP1 and MCF on kidney burdens in mice infected with and mutant strain DPL22 S645P/S at 24 h and 48 h post-inoculation.

FIG. 2A shows CMP1 and MCF inhibition profiles of enriched 1,3-β-D-glucan synthase from susceptible and resistant C. albicans and C. glabrata isolates.

FIG. 2B shows a scatter plot used to determine the MPC values of CMP1 for wild-type C. albicans and C. glabrata isolates.

FIG. 2C shows a scatter plot used to determine the MPC values of MCF for wild-type C. albicans and C. glabrata isolates.

FIG. 3A shows a scatter plot demonstrating that reduced susceptibility was observed for ANID in Candida strain C. albicans NRRL Y-447 following 20 serial passages.

FIG. 3B shows a scatter plot demonstrating that reduced susceptibility was observed for CMP1, ANID, and CAS in Candida strain C. glabrata ATCC 90030 following 20 serial passages.

FIG. 3C shows a scatter plot demonstrating that reduced susceptibility was observed for CMP1, ANID, and CAS in Candida strain C. glabrata ATCC 2001 following 20 serial passages.

FIG. 3D shows a scatter plot demonstrating that reduced susceptibility was observed for CMP1, and CAS in Candida strain C. parapsilosis CP02 following 20 serial passages.

FIG. 3E shows a scatter plot demonstrating that reduced susceptibility was observed for CAS in Candida strain C. krusei ATCC 6258 following 20 serial passages.

FIG. 4 shows the antifungal activities of caspofungin, anidulafungin, micafungin, fluconazle, and voriconazole tested against different species of Candida and Aspergillus fumigatus.

FIG. 5 shows the fungicidal properties of Compound 1 against C. albicans ATCC 44858.

FIG. 6 shows that Compound 1 in a gel formulation is highly efficacious against azole-resistant Candida albicans in a rat model of vulvovaginal candidiasis.

FIGS. 7A-7D show the fungicidal properties of Compound 1 and terconazole (TCZ) against azole-susceptible strain C. albicans ATCC 4458 and the fungicidal properties of Compound 1 against azole-resistant strains C. albicans DPL001 and R357.

FIGS. 8A-8D show the fungicidal properties of Compound 1 and TCZ against azole-susceptible strain C. glabrata CG01 and the fungicidal properties of Compound 1 against azole-resistant strains C. glabrata ATCC 200918 and MMX 7070.

FIGS. 9A-9D show the fungicidal properties of Compound 1 and TCZ against azole-susceptible strain C. tropicalis CTO2 and the fungicidal properties of Compound 1 against azole-resistant strains C. tropicalis MMX 7255 and MMX 7525.

FIGS. 10A-10D show the fungicidal properties of Compound 1 and TCZ against azole-susceptible strain C. parapsilosis CP02 and the fungicidal properties of Compound 1 against azole-resistant strains C. parapsilosis CP01 and MMX 7370.

FIGS. 11A-11B show the fungicidal properties of Compound 1 against azole-susceptible strain C. Krusei ATCC 6258 and against azole-resistant strain C. Krusei ATCC 14243.

FIG. 12 shows kidney burdens in mice infected with different inoculum densities of azole-resistant C. albicans strain R357.

FIG. 13 shows an outline of the experimental protocol used to evaluate the efficacy of Compound 1, amphotericin B, and fluconazole in a C. albicans R357 infection model.

FIGS. 14A and 14B show effects of Compound 1 (CMP1), amphotericin B (AM-B), and fluconazole (FLU) on kidney burdens in mice infected with azole-resistant C. albicans strain R357.

FIG. 15 shows pharmacokinetics of Compound 1 over doses 1 mg/kg, 4 mg/kg, and 16 mg/kg.

FIG. 16 shows net change in fungal density (logio CFU) versus different total doses of Compound 1 at different fractionation schedules.

FIG. 17 shows change in fungal density (logio CFU) reduction from baseline caused by 2 mg/kg total dose of Compound 1 at different fractionation schedules.

FIG. 18 shows simulated free-drug concentration time profiles relative to the MIC for the fractionated Compound 1 2 mg/kg regimen.

FIG. 19 shows the percentage of survival in mouse infection models of disseminated aspergillosis that are treated with Compound 1 and amphotericin B.

FIG. 20 shows pharmacokinetics and target attainment of single and multiple doses of Compound 1 administered intravenously.

### DETAILED DESCRIPTION

Provided are methods of treating a fungal infection in a subject (e.g., a human) by administering to the subject a salt of Compound 1, or a neutral form thereof. In some embodiments, the subject has failed treatment with an antifungal therapy, such as an echinocandin therapy, a polyene therapy, flucytosine therapy, and/or an azole therapy. The inventors have found that Compound 1 displays long-acting pharmacokinetics with a long half-life and slow clearance and strong activities against both wild-type and mutant 1,3-β-D-glucan synthase enzyme complex, and can suppress the emergence of resistant fungal strains at certain dosing levels and frequencies.

### I. Fungal Infection

Fungal infections that can be treated by the methods described herein include, but are not limited to, drug-resistant fungal infections (e.g., an echinocandin-resistant fungal infection, a polyene-resistant fungal infection, a flucytosine-resistant fungal infection, and an azole-resistant fungal infection). In some embodiments, the fungal infection may be caused by a fungus having one or more mutations in the 1,3-β-D-glucan synthase enzyme complex. In some embodiments, the fungal infection may be caused by a fungus having one or more mutations in the FKS genes. In some embodiments, the fungal infection or the subject (e.g., a human) having the fungal infection may have failed treatment with an echinocandin therapy, a polyene therapy, flucytosine therapy, and/or an azole therapy. In some embodiments, the subject has failed treatment with other antifungal agents and/or 1,3-β-D-glucan synthase inhibitors, such as enfumafungin and SCY-078. In some embodiments, the fungal infections that can be treated by the methods described herein are caused by fungi from the genus Candida, Aspergillus, and/or dermatophytes. In some embodiments, the fungal infection being treated can be an infection selected from tinea capitis, tinea corporis, tinea pedis, onychomycosis, perionychomycosis, or pityriasis versicolor.

### Candida Infection

A Candida infection refers to an infection caused by a fungus in the genus Candida. Fungi display several adaptive physiological mechanisms that result in their resistance to echinocandins. Echinocandin-resistant Candida infections do not respond to treatment with echinocandins, which may display highly elevated minimal inhibition concentrations (MIC) against the echinocandin-resistant Candida. As described herein, the echinocandins referred to in the term “echinocandin-resistant Candida infection” are cyclic lipopeptides, such as micafungin, caspofungin, and anidulafungin, that inhibit the synthesis of glucan in the cell wall by inhibition of the catalytic unit of the 1,3-β-D-glucan synthase enzyme complex. The echinocandins referred to in the term “echinocandin-resistant Candida infection” do not include Compound 1.

Echinocandins target the cell wall by preventing the production of 1,3-β-D-glucan through inhibition of the catalytic subunit of the 1,3-β-D-glucan synthase enzyme complex. The major mechanism of echinocandin-resistance is related to mutations in the FKS genes (Fks1, Fks2, and Fks3) that encode the catalytic subunit of 1,3-β-D-glucan synthase enzyme complex. Resistance to echinocandins is associated with mutations in two hot spot (HS) regions in the FKS genes that correlate with clinical failure or poor response to echinocandin therapy. In some embodiments, mutations in FKS genes occur spontaneously, i.e., spontaneous mutations. Spontaneous mutations may arise from a variety of sources, e.g., errors in DNA replication, spontaneous lesions, and transposable genetic elements. In some embodiments, spontaneous mutations may confer drug resistance in a fungus in the genus Candida. For example, a Candida fungus may be initially susceptible to an echinocandin (e.g., micafungin, caspofungin, anidulafungin), however, as the Candida fungus grows and replicates over a period of time, spontaneous mutations may occur in the Candida fungus that confer resistance in the Candida fungus against the echinocandin (e.g., micafungin, caspofungin, anidulafungin). As described herein, a Candida infection may an echinocandin-resistant Candida infection if the Candida fungus causing the infection develops spontaneous mutations that allow the fungus to be resistant to one or more echinocandins (e.g., micafungin, caspofungin, anidulafungin). In some embodiments, a Candida fungus may develop spontaneous mutations in the presence or absence of an echinocandin (e.g., micafungin, caspofungin, anidulafungin).

Prominent mutations in FKS genes decrease the sensitivity of 1,313-D-glucan synthase for echinocandins (e.g., micafungin, caspofungin, and anidulafungin) significantly, and Candida strains harboring such mutations may require a concomitant increase in drug dose to reduce fungal organ burdens in animal infection models. The mutations in the FKS genes are genetically dominant, conserved in a wide variety of Candida spp., and confer cross-resistance to echinocandins (e.g., micafungin, caspofungin, and anidulafungin).

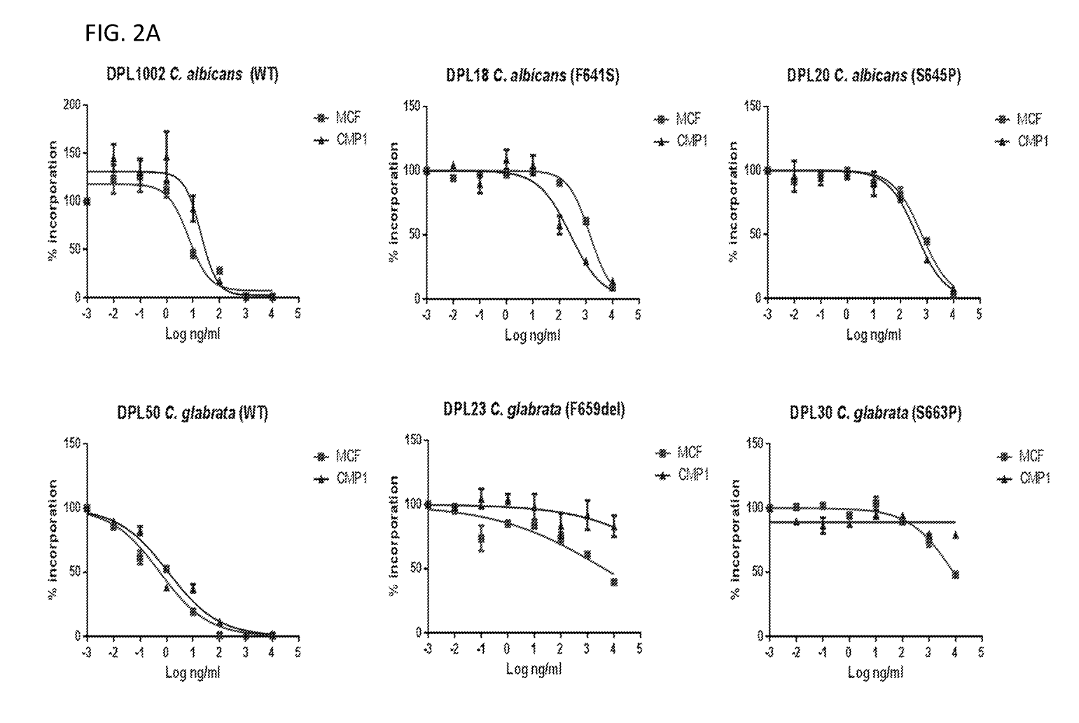

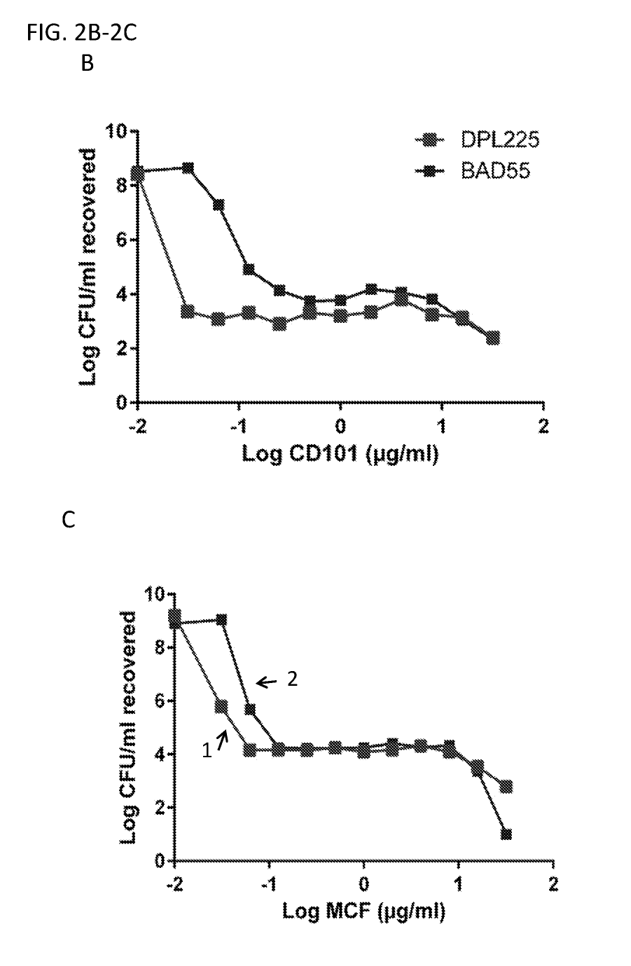

As described in detail herein (see, e.g., Examples), Compound 1 displays long-acting pharmacokinetics with a long half-life and slow clearance and strong activities against both wild-type and mutant 1,3-β-D-glucan synthase enzyme complex. For example, in some embodiments, the Candida spp. causing the Candida infection (e.g., an echinocandin-resistant Candida infection) has a mutant 1,3-β-D-glucan synthase enzyme complex that has mutations in the FKS genes. In some embodiments, a mutant 1,3-β-D-glucan synthase enzyme complex has one or more amino acid mutations listed in Table 1. Additional mutations in FKS genes that can impart anti-fungal resistance are described in, e.g., Perlin D. Drugs 74:1573-1585, 2014 (see, e.g., FIGS. 2A and 2B of Perlin)

TABLE 1

| | | | | |
|---|---|---|---|---|
| Fks1 HS1 | S629P | D632Y | F641S | S645Y |
| | | | | S645P |
| Fks1 HS2 | I1366S | | | |
| Fks2 HS1 | F659del | S663F | R665G | D666I |
| | F659I | S663P | | D666H |
| | F659S | | | D666Y |
| | | | | D666N |
| Fks2 HS2 | R1378S | | | |

### II. Methods of Treating a Fungal Infection Using a Salt of Compound 1, or a Neutral Form Thereof

Compound 1 exhibits long-acting pharmacokinetics with a long half-life and slow clearance and strong activities against both wild-type and mutant 1,3-β-D-glucan synthase enzyme complex.

The disclosure includes methods for treating a drug-resistant fungal infection in a subject (e.g., a human) by administering a salt of Compound 1, or a neutral form thereof, to the subject. The disclosure also features methods of treating fungal infections by administering a salt of Compound 1, or a neutral form thereof, to the subject (e.g., a human) who has failed treatment with an antifungal therapy, such as an echinocandin therapy, a polyene therapy, flucytosine therapy, or an azole therapy. In some embodiments, the subject has failed treatment with other antifungal agents and/or 1,3-β-D-glucan synthase inhibitors, such as enfumafungin and SCY-078. In some embodiments, the fugal infection treatable by the methods described herein is caused by a fungus having a mutant 1,3-β-D-glucan synthase enzyme complex. In some embodiments, the mutant 1,3-β-D-glucan synthase enzyme complex contains mutations in the FKS gene. In some embodiments, the administering step includes intravenously administering about 150 mg to about 800 mg of a salt of Compound 1, or a neutral form thereof, administered in two or more doses to the subject over a period of 1 to 4 weeks.

In some methods of treating a fungal infection described herein, the methods include intravenously administering to a subject about 550 mg to about 800 mg of a salt of Compound 1, or a neutral form thereof, in which the salt of Compound 1, or the neutral form thereof is administered in two or more doses to the subject over a period of 1 to 4 weeks. In some methods, the methods include intravenously administering to the subject about 150 mg to about 800 mg of a salt of Compound 1, or a neutral form thereof, in which the salt of Compound 1, or the neutral form thereof is administered to the subject one to three times per week to the subject for 2 to 4 weeks. In some methods, the methods include intravenously administering to a subject two or more doses of a composition that contains about 150 mg to about 800 mg of a salt of Compound 1, or a neutral form thereof, in which the two or more doses are administered every three to eight days. In some embodiments, the administered amount maintains at least a mutant prevention concentration of Compound 1 in plasma during treatment of the fungal infection.

In some embodiments, the fungal infection is an echinocandin-resistant, polyene-resistant, flucytosine-resistant, or azole-resistant fungal infection. In some embodiments, the fungal infection is an echinocandin-resistant infection. In some embodiments, the subject receiving the therapeutic methods described herein has failed treatment with an echinocandin therapy, a polyene therapy, flucytosine therapy, or an azole therapy (e.g., an echinocandin therapy). In some embodiments, the subject has failed treatment with anidulafungin, micafungin, or caspofungin. In some embodiments, the subject has failed treatment with other antifungal agents and/or 1,3-β-D-glucan synthase inhibitors, such as enfumafungin and SCY-078.

In some embodiments, the fungal infection is caused by a fungus having a mutant 1,3-β-D-glucan synthase enzyme complex having one or more mutations in FKS genes. In some embodiments, the fungal infection is a Candida infection. In some embodiments, a Candida infection can be caused by a fungus in the genus Candida that is selected from the group consisting of Candida albicans, C. glabrata, C. dubliniensis, C. krusei, C. parapsilosis, C. tropicalis, C. orthopsilosis, C. guilliermondii, C. rugosa, and C. lusitaniae. In some embodiments, a Candida infection can be caused by an antifungal drug-susceptible or antifungal drug-resistant strain of fungus in the genus Candida, such as an antifungal drug-susceptible or antifungal drug-resistant strain of any one of Candida albicans, C. glabrata, C. dubliniensis, C. krusei, C. parapsilosis, C. tropicalis, C. orthopsilosis, C. guilliermondii, C. rugosa, and C. lusitaniae. Antifungal drugs include, but are not limited to, echinocandins, polyene compounds, flucytosine, and azole compounds. In some embodiments, a Candida infection can be caused by an azole-susceptible or azole-resistant strain of fungus in the genus Candida, such as an azole-susceptible or azole-resistant strain of any one of Candida albicans, C. glabrata, C. dubliniensis, C. krusei, C. parapsilosis, C. tropicalis, C. orthopsilosis, C. guilliermondii, C. rugosa, and C. lusitaniae. In some embodiments, the Candida is Candida albicans. In some embodiments, an azole-resistant strain of fungus is Candida albicans, e.g., Candida albicans R357 strain. Azole-resistant Candida albicans R357 strain contains mutations in the gene ERG11 (e.g., Candida albicans ERG11 (CaERG11)). The CaERG11 gene encodes the enzyme 14α-demethylase, the target of azole antifungal compounds. Mutations in the CaERG11 gene that result in amino acid substitutions alter the abilities of the azole compounds to bind to and inhibit 14α-demethylase, thus resulting in resistance. In some embodiments, an azole-resistant Candida albicans R357 strain have an increase in CaERG11 expression, e.g., 2-15 times (e.g., 3-15, 4-15, 5-15, 6-15, 7-15, 8-15, 9-15, 10-15, 11-15, 12-15, 13-15, or 14-15 times) more increased expression relative to a wild-type strain. In some embodiments, an azole-resistant Candida albicans R357 strain have one or more mutations in the CaERG11 gene that lead to one or more amino acid substitutions, e.g., D116E, D153E, and/or E266D. In some embodiments, an azole-resistant Candida albicans R357 strain have no significant changes in CDR1 or MDR1 expression. Table 2 shows the percentage of inhibition and MIC values of three azole compounds, amphotericin B, caspofungin, and Compound 1 towards the azole-resistant Candida albicans R357 strain and susceptibility status (S: susceptible; R: resistant) as classified by CLSI (Clinical and Laboratory Standards Institute) of the Candida albicans R357 strain towards the listed compounds.

TABLE 2

| Antifungal | Endpoint | MIC | Susceptibility |
|---|---|---|---|
| agent | (% inhibition) | (μg/mL) | (CLSI) |
| Fluconazole | 50% | >64 | R |
| Voriconazole | 50% | >64 | R |
| Posaconazole | 50% | >64 | |
| Amphotericin B | 100% | 0.5 | S |
| Caspofungin | 50% | 0.25 | S |
| Compound 1 | 50% | 0.125 | |

In some embodiments, the Candida is Candida glabrata. In some embodiments, the Candida infection is candidemia, oropharyngeal candidiasis, esophageal candidiasis, mucosal candidiasis, genital candidiasis, vulvovaginal candidiasis, gastrointestinal candidiasis, rectal candidiasis, hepatic candidiasis, renal candidiasis, pulmonary candidiasis, splenic candidiasis, cardiovascular candidiasis (e.g., endocarditis), or invasive candidiasis.

In addition to a Candida infection, a fungal infection that can be treated by the therapeutic methods described herein can also be an Aspergillus infection. In some embodiments, an Aspergillus infection is caused by a fungus the genus Aspergillus, e.g., Aspergillus fumigatus, A. flavus, A. terreus. A. niger, A. candidus, A. clavatus, or A. ochraceus. In some embodiments, an Aspergillus infection is caused by Aspergillus fumigatus. In some embodiments, the Aspergillus infection is aspergillosis (e.g., invasive aspergillosis, central nervous system aspergillosis, or pulmonary aspergillosis). In some embodiments, a fungal infection may also be a dermatophyte infection, which can be caused by a fungus in the genus Microsporum, Epidermophyton, and Trichophyton.

In some embodiments of the therapeutic methods described herein, the administering step includes administering a salt of Compound 1, or a neutral form thereof, topically, intravaginally, intraorally, intravenously, intramuscularly, intradermally, intraarterially, subcutaneously, orally, or by inhalation. In some embodiments, a salt of Compound 1, or a neutral form thereof, is administered intravenously. The disclosure also features methods of killing an echinocandin-resistant, polyene-resistant, flucytosine-resistant, or azole-resistant Candida comprising exposing the echinocandin-resistant, polyene-resistant, flucytosine-resistant, or azole-resistant Candida to a salt of Compound 1, or a neutral form thereof, in an amount and for a duration sufficient to kill the echinocandin-resistant, polyene-resistant, flucytosine-resistant or azole-resistant Candida. In some embodiments, Candida is Candida albicans or Candida glabrata.

### III. Pharmaceutical Compositions and Preparations

Compound 1 may be prepared in a pharmaceutical composition. In some embodiments, the pharmaceutical composition includes a salt of Compound 1, or a neutral form thereof, and pharmaceutically acceptable carriers and excipients. Depending on the mode of administration and the dosage, Compound 1 used in the methods described herein will be formulated into suitable pharmaceutical compositions to permit facile delivery. Compound 1 may be formulated in a variety of ways that are known in the art. For use as treatment of human and animal subjects, Compound 1 can be formulated as pharmaceutical or veterinary compositions. Depending on the subject (e.g., a human) to be treated, the mode of administration, and the type of treatment desired, e.g., prevention, prophylaxis, or therapy, Compound 1 is formulated in ways consonant with these parameters. A summary of such techniques is found in Remington: The Science and Practice of Pharmacy, 22^{nd} Edition, Lippincott Williams & Wilkins, (2012); and Encyclopedia of Pharmaceutical Technology, eds. J. Swarbrick and J. C. Boylan, 2006, Marcel Dekker, New York, each of which is incorporated herein by reference. p Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol.

The pharmaceutical compositions can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Gibson (ed.) Pharmaceutical Preformulation and Formulation (2nd ed.) Taylor & Francis Group, CRC Press (2009).

The pharmaceutical compositions can be prepared in the form of an oral formulation. Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

The pharmaceutical composition may be formed in a unit dose form as needed. The amount of active component, e.g., Compound 1, included in the pharmaceutical compositions are such that a suitable dose within the designated range is provided (e.g., a dose within the range of 0.01-100 mg/kg of body weight).

### IV. Routes, Dosage, and Administration

Compound 1 or pharmaceutical compositions including Compound 1 may be formulated for, e.g., topical administration, intravaginal administration, intraoral administration, intravenous administration, intramuscular administration, intradermal administration, intraarterial administration, subcutaneous administration, oral administration, or by inhalation.

In some embodiments, Compound 1 or pharmaceutical compositions including Compound 1 may be formulated for intravenous administration. For injectable formulations, various effective pharmaceutical carriers are known in the art. See, e.g., Remington: The Science and Practice of Pharmacy, 22^{nd} ed., (2012) and ASHP Handbook on Injectable Drugs, 18^{th} ed., (2014).

The dosage of Compound 1 or the pharmaceutical composition depends on factors including the route of administration, the infection to be treated, and physical characteristics, e.g., age, weight, general health, of the subject (e.g., a human). The dosage may be adapted by the physician in accordance with conventional factors such as the extent of the disease and different parameters of the subject. Typically, the amount of Compound 1 or the pharmaceutical composition contained within a single dose may be an amount that effectively prevents, delays, or treats the infection without inducing significant toxicity.

In some embodiments of the methods described herein, the administering step includes intravenously administering about 150 mg to about 800 mg of a salt of Compound 1, or a neutral form thereof, to a subject having a fungal infection in two or more doses over a period of 1 to 4 weeks.

In some methods, a salt of Compound 1, or a neutral form thereof, is intravenously administered to a subject in about 550 mg to about 800 mg in two or more doses over a period of 1 to 4 weeks. In some methods, a salt of Compound 1, or a neutral form thereof, is administered intravenously to a subjection in about 150 mg to about 800 mg of one to three times per week for 2 to 4 weeks. In some methods, the methods include intravenously administering two or more doses of a composition containing about 150 mg to about 800 mg of a salt of Compound 1, or a neutral form thereof, to a subject, wherein the two or more doses are administered every three to eight days.

In some methods, Compound 1 in salt or neutral form is intravenously administered to a subject in two or more weekly doses (e.g., 2, 3, 4, 5, 6, 7, 8, 9, 10, 11, or 12 doses), wherein the first dose contains about 400 mg of Compound 1 in salt or neutral form and each of the subsequent doses contains about 200 mg of Compound 1 in salt or neutral form. In some embodiments, the first dose includes about 400 mg of Compound 1 in salt or neutral form, and each of the remaining doses includes about 200 mg of Compound 1 in salt or neutral form. In some embodiments, the dosing regimen consists of (a) intravenously administering a first dose of about 400 mg of Compound 1 in salt or neutral form, (b) intravenously administering a second dose of about 200 mg of Compound 1 in salt or neutral form, and (c) optionally intravenously administering a third dose of about 200 mg of Compound 1 in salt or neutral form, wherein the first dose is administered on day 1, the second dose is administered on day 8, and the third dose, if administered, is administered on day 15.

In some methods, Compound 1 in salt or neutral form is intravenously administered to a subject (e.g., a human) in two or more weekly doses (e.g., 2, 3, 4, 5, 6, 7, 8, 9, 10, 11, or 12 doses) of about 400 mg of Compound 1 in salt or neutral form. In some embodiments, the dosing regimen consists of (a) intravenously administering a first dose of about 400 mg of Compound 1 in salt or neutral form, (b) intravenously administering a second dose of about 400 mg of Compound 1 in salt or neutral form, and (c) optionally intravenously administering a third dose of about 400 mg of Compound 1 in salt or neutral form, wherein the first dose is administered on day 1, the second dose is administered on day 8, and the third dose, if administered, is administered on day 15.

As used herein, the amount in each dose refers to the amount of Compound 1 (structure of Formula I shown above) that does not include the negative counterion (e.g., an acetate) if Compound 1 is in its salt form. For example, a dose of about 400 mg or 200 mg of Compound 1 in salt or neutral form refers to 400 mg or 200 mg of Compound 1, not including the acetate ion if Compound 1 is in an acetate salt form.

In some embodiments, the third dose of about 200 mg of Compound 1 in salt or neutral form is administered if on day 15 the subject displays symptoms of a fungal infection. In some embodiments, the third dose of about 400 mg of Compound 1 in salt or neutral form is administered if on day 15 the subject displays symptoms of a fungal infection. In some embodiments, symptoms of the fungal infection includes fever, cough, shortness of breath, weight loss, and/or night sweats.

In some embodiments of the methods described herein, Compound 1 in salt or neutral form is administered for 2-12 doses (e.g., 2-3 doses). In some embodiments, Compound 1 in salt or neutral form is administered until mycological eradication and/or clinical cure is achieved as determined by a standard test known in the art. In some embodiments, mycological eradication is defined as two negative blood cultures drawn at 12 hours apart without intervening positive blood cultures and no change of antifungal therapy for the fungal infection. In some embodiments, Compound 1 in salt or neutral form is administered until the subject is free of symptoms of the fungal infection, such as fever, cough, shortness of breath, weight loss, and night sweats, as determined by a physician. In some embodiments, the amount of Compound 1 in salt or neutral form is administered for a duration sufficient to treat the fungal infection. In some embodiments, the fungal infection may be a drug-resistant fungal infection, an echinocandin-resistant fungal infection, a polyene-resistant fungal infection, a flucytosine-resistant fungal infection, and/or an azole-resistant fungal infection. In some embodiments, the fungal infection may be caused by a fungus having one or more mutations in the 1,3-β-D-glucan synthase enzyme complex. In some embodiments, the fungal infection may be caused by a fungus having one or more mutations in the FKS genes.

Compound 1 or pharmaceutical compositions containing Compound 1 are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, and oral dosage forms (e.g., ingestible solutions, drug release capsules). Compound 1 or pharmaceutical compositions may be in the form of, e.g., tablets, capsules, pills, powders, granulates, suspensions, emulsions, solutions, gels including hydrogels, pastes, ointments, creams, plasters, drenches, osmotic delivery devices, suppositories, enemyas, injectables, implants, sprays, preparations suitable for iontophoretic delivery, or aerosols. The compositions may be formulated according to conventional pharmaceutical practice. The timing between administrations may decrease as the medical condition improves or increase as the health of the patient declines.

The following Examples are intended to further illustrate but not to limit the disclosure herein.

### EXAMPLES

### Example 1

### Efficacy of Compound 1 to Treat Echinocandin-Resistant Candida albicans in a Murine Model of Invasive Candidiasis

In this experiment, we evaluated the in vivo efficacy of Compound 1 (CMP1) against echinocandin-susceptible and -resistant C. albicans strains in an invasive candidiasis murine model. Female 6-week-old BALB/c mice weighing 18-22 g were rendered neutropenic by receiving 150 mg/kg and 100 mg/kg of cyclophosphamide via IP injection on day -4 and day -1 prior to infection, respectively.

Two C. albicans strains, ATCC 90028 (FKS WT) and DPL22 (fks/FKS mutant S645P/S), were used in this experiment. ATCC 90028 is an FKS wild-type strain and sensitive to echinocandin drugs. DPL22 is a heterozygous fks/FKS mutant (S645P/S) strain. The organisms were subcultured in liquid yeast extract-peptone-dextrose (YPD) medium at 37° C. with shaking overnight. Cells were collected by centrifugation, washed twice with sterile phosphate-buffered saline (PBS), and counted with a hemocytometer. The inoculum was adjusted to 5×10⁶ colony-forming units (CFU)/mL and 100 μI was used to infect each mouse. Actual infection dose was verified by viable counts on YPD plates spread with proper dilutions of the inoculum and incubated at 37° C. for 24 h.

On day 0, mice were infected with 5×10⁵ CFU of C. albicans FKS wild-type (ATCC 90028) (n=50) or heterozygous mutant strain (DPL22 S645P/S) (n=50) via lateral tail vein injection. Groups consisting of 10 mice were given single doses of vehicle, Compound 1 at 20 mg/kg, 40 mg/kg, or 60 mg/kg, or antifungal control (micafungin (MCF), 5 mg/kg, equivalent to clinical therapeutic dose) at 3 h post-infection via IP injection. At 24 h post-inoculation and at the experiment endpoint 48 h post-inoculation, 5 mice from each group were euthanized via CO₂ inhalation and kidneys were aseptically removed for enumeration of fungal burdens.

All graphic data are expressed as means±SD and were statistically analyzed by analysis of variance (ANOVA) using computer Prism software (Prism 5; GraphPad Software, Inc., San Diego, Calif.). A P value of <0.05 is considered statistically significant.

Table 3 shows the minimal inhibitory concentration (MIC) and glucan synthase half maximal inhibitory concentration (IC₅₀) values for both micafungin (MCF) and Compound 1 (CMP1) against fungal strains ATCC 90028 (FKS WT) and DPL22 (fks/FKS mutant S645P/S). Both micafungin (MCF) and Compound 1 (CMP1) display elevated MIC and IC₅₀ values against fungal strain DPL22.

TABLE 3
Strains used in mouse study

| | | Fksp | | MIC | | | |
|---|---|---|---|---|---|---|---|
| | | Phenotype | | (μg/mL) | | IC₅₀ (ng/mL) | |
| Strain | Organism | Fks1p | Fks2p | MCF | CMP1 | MCF | CMP1 |
| ATCC 90028 | C. albicans | WT | WT | <0.03 | <0.03 | 18 | 14 |
| DPL22 | C. albicans | S645P/S | WT | 0.5 | 0.5 | 245 | 30 |

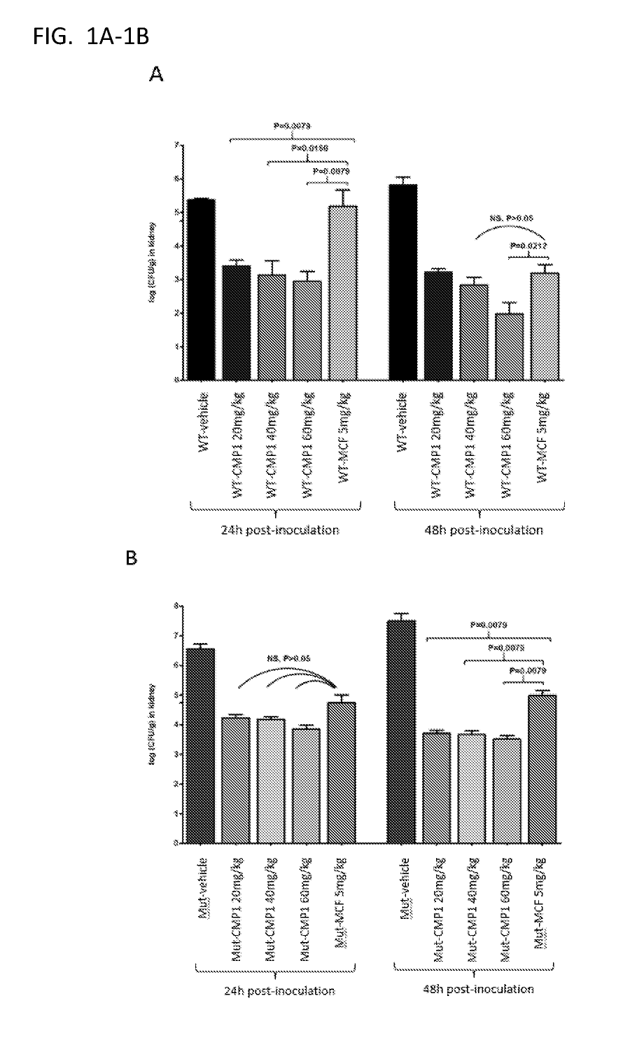

Compound 1 (CMP1) at all three doses exhibited strong activities against both wild-type and fks/FKS mutant strains of C. albicans, as demonstrated by significant kidney burden reduction in all treatment groups at both 24 h and 48 h post-inoculation time points (P<0.05) (FIGS. 1A and 1B). FIG. 1A shows that in wild-type strain infected mice, CMP1 exhibited better efficacy than micafungin at 24 h post-inoculation at all three doses. Although the superiority of CMP1 relative to MCF was only seen with the highest dose (60 mg/kg) at 48 h post-inoculation, the efficacy of CMP1 at 20 mg/kg and 40 mg/kg was still comparable with micafungin at 5 mg/kg.

Regarding the fks/FKS mutant strain (DPL22) infected mice, CMP1 treatment significantly reduced kidney burdens by over 2 logs at 24 h post-inoculation compared to vehicle control (P<0.05). The 24 h burden reduction was not significantly different among three CMP1 dosage groups or MCF treatment group. However, better efficacy of CMP1 compared to MCF at 5 mg/kg was observed for all three doses at 48 h post-inoculation. Burden reduction was comparable in three CMP1 groups (FIG. 1B).

### Example 2

### Minimal Inhibitory Concentration (MIC) Values and Mutant Prevention Concentration (MPC) Values of Micafungin (MCF) and Compound 1 (CMP1) Against Echinocandin-Susceptible and Resistant Candida spp.

This experiment characterizes the in vitro activity of Compound 1 (CMP1) through assessment of enzymatic inhibition of wild-type and mutant β-(1,3)-glucan synthase (GS), MIC values against well-characterized echinocandin-susceptible and resistant isolates and mutant prevention concentration (MPC) in Candida spp.

Glucan Synthase (GS) purification and assay. Three C. albicans strains (DPL1002, DPL18, DPL20) and three C. glabrata strains (DPL50, DPL23, DPL30) were grown with vigorous shaking at 37° C. to early stationary phase in YPD (1% Yeast extract, 2% Peptone, 2% Dextrose) broth, and cells were collected by centrifugation. Cell disruption, membrane protein extraction and partial 1,3-β-D-glucan synthase purification by product-entrapment were performed as described in Park et al., Antimicrob. Agents Chemother. 49:3264-3273, 2005. The kinetic inhibition parameter IC₅₀ (half-maximal inhibitory concentration) was determined for GS extracted from wild-type and fks mutant strains by measuring incorporation of radiolabeled glucose from UDP-[¹⁴C]-glucose into ethanol-insoluble polymeric products. Sensitivity to micafungin (MCF) and Compound 1 (CMP1) was measured in a polymerization assay using a 96-well 0.65 μm multiscreen HTS filtration system (Millipore Corporation, Bedford, Mass.) in a final volume of 100 μl, as previously described Garcia-Effron et al., Antimicrob. Agents Chemother. 53:3690-3699, 2009. Serial dilutions of the drugs (0.001-10,000 ng/mL) were used to determine IC₅₀ values. MCF was dissolved in water and CMP1 was dissolved in 100% dimethyl sulfoxide (DMSO). Reactions were initiated by addition of glucan synthase. Inhibition profiles and half maximal inhibitory concentrations (IC₅₀s) were determined using a normalized response (variable-slope) curve fitting algorithm with GraphPad Prism, version 5.04, software (Prism Software, Irvine, Calif.).

Antifungal susceptibility testing. Antifungal susceptibility testing was performed in triplicate for a collection of 95 Candida strains (20 C. albicans, 20 C. glabrata, 2 C. dubliniensis, 15 C. krusei, 19 C. parapsilosis, and 19 C. tropicalis) that included 30 isolates showing an echinocandin resistance (ER) phenotype (caspofungin-resistant) in accordance with the guidelines described in the Clinical and Laboratory Standards Institute (CLSI) document M27-S4. MCF was dissolved in water and CMP1 was dissolved in 100% DMSO. Stock solutions of the drugs were kept at −86° C. Microtiter plates were read visually at 24 and 48 hr and the MIC determined using prominent inhibition (corresponding to 50%) as endpoint.

Mutant prevention concentration (MPC) determination. The MPC represents a threshold above which the selective proliferation of resistant mutants is expected to occur only rarely. It is based on the concept that mutants are selected within a concentration range that extends from the MIC up to the MPC (see, e.g., Drlica et al., Clin. Infect. Dis. 44:681-8, 2007 and Drlica et al., J Antimicrob. Chemother. 52:11-17,2003). Strains were grown overnight in YPD (1% Yeast extract, 2% Peptone, 2% Dextrose) broth with vigorous shaking at 37° C. Cells were collected by centrifugation and washed 1× with distilled water. Samples were diluted to 1×10⁸ CFU/mL in a total volume of 1.5 mL. One hundred microliters of fungal suspension was added to 0.9 mL of RPMI 1640 medium buffered with MOPS to pH 7.0 with or without drug, providing the starting inoculum of approximately 1×10⁷ CFU/mL. The range of CMP1 or MCF concentrations tested was 0.03-32 μg/mL. The culture vials were incubated with agitation at 37° C. for 24 hr. A 100 μl sample was removed from each culture vial and serially diluted with sterile water. Subsequently, 100 μl aliquots of several dilutions were plated on YPD. When colony counts were suspected to be low, 100 μl was taken directly from the culture vials and plated without dilution. Plates were incubated at 37° C. for 1-2 days prior to colony counting.

Inhibition curves for CMP1 and MCF against the wild-type (WT) C. albicans isolates showed the typical pattern of β-(1,3)-glucan synthase echinocandin susceptibility (FIG. 2A and Table 4 below). The F641 S mutant exhibited a 100-fold and 24.3-fold increase in ICso values for MCF and CMP1, respectively, compared to the WT. The S645P mutant exhibited 144-fold and 185.3-fold increases for MCF and CMP1, respectively. Mean IC₅₀ values for the WT C. glabrata enzyme were 0.447 and 2.57 ng/mL for MCF and CMP1, respectively. The C. glabrata F659del mutant GS did not exhibit significant reductions in activity after treatment with a high dose (10,000 ng/mL) of either MCF or CMP1. The S663P mutant exhibited a lower ICso for MCF compared to CMP1. CMP1 is a potent inhibitor of glucan synthase with comparable activity to other echinocandins against Candida spp. CMP1 presented similar IC₅₀ values as MCF against the WT and fks mutant strains of C. albicans and C. glabrata analyzed, although it showed enhanced activity relative to MCF against the common Fks1-F641S mutant enzyme from C. albicans.

TABLE 4
IC₅₀s of β-(1,3)-glucan synthases from susceptible and resistant
C. albicans and C. glabrata isolates used in the experiment.

| | | Fks1 | Fks2 | IC₅₀ (ng/mL) | |
|---|---|---|---|---|---|
| DPL# | Organism | Phenotype | Phenotype | MCF | CMP1 |
| 1002 | C. albicans | WT | WT | 17.65 | 14.25 |
| 18 | C. albicans | F641S | | 1782 | 347.4 |
| 20 | C. albicans | S645P | | 2555.77 | 2641.4 |
| 50 | C. glabrata | WT | WT | 0.447 | 2.57 |
| 23 | C. glabrata | | F659del | >10,000 | >10,000 |
| 30 | C. glabrata | | S663P | 6772.33 | >10,000 |

CMP1 did not show significant differences in MIC values for the wild-type (WT) isolate population relative to MCF with the exception of C. krusei isolates, which were 2- to 4-fold lower for CMP1 than MCF. Although MIC values were comparable for both CMP1 and MCF for the different Candida ER isolates, 60% of C. albicans ER isolates had an MIC of ≤0.5 mg/L for CMP1 compared to 40% for MCF (Table 5). However, only 18% (2/11) of C. glabrata ER isolates had an MIC of ≤5 mg/L for CMP1 compared to 63% (7/11) for MCF. This finding does not appear to be genotype-dependent, as mutations in either FKS1 or FKS2 showed comparable results with MCF. CMP1 MICs were 1- to 2-fold lower compared to MCF for C. krusei ER isolates.

TABLE 5
In vitro whole-cell susceptibility (MIC) distributions of MCF and
CMP1 for the Candida isolates included in this experiment.

| | Phenotype | | |
|---|---|---|---|
| | (no. of | MIC median (range), mg/L^{a} | |
| Species | isolates) | MCF | CMP1 |
| C. albicans | WT (10) | <0.03 (<0.03) | <0.03 (<0.03) |
| | ER (10) | 0.50 (0.03-4) | 0.50 (0.06-2) |
| C. glabrata | WT (9) | <0.03 (<0.03) | 0.06 (0.03-0.12) |
| | ER (11) | 0.50 (0.06-4) | 1.00 (0.12-4) |
| C. | WT (1) | 0.03 | 0.03 |
| dubliniensis | ER (1) | 0.03 | 0.03 |
| C. krusei | WT (11) | 0.12 (0.03-0.25) | 0.03 (<0.03-0.06) |
| | ER (4) | 1.00 (0.03-8) | 0.50 (<0.03-4) |
| C. | WT (19) | 4 (1-4) | 2 (2-4) |
| parapsilosis | | | |
| C. tropicalis | WT (15) | 0.03 (0.03) | 0.03 (0.03) |
| | ER (4) | 2.00 (1-4) | 2.00 (0.5-2) |

^{a}Data represent median MIC values and ranges after 24 hr of growth at 37° C. MIC values at 48 hr were the same or within a 2-fold range of the 24 hr value. All values represent averages of the results of triplicate experiments.

To measure MPCs, we used a modified version of the original method as described in Zhao et al., Clin Infect Dis 33 Suppl 3:S147-156, 2001. We observed a 3- to 5-log decrease in CFUs around the MICs for CMP1 (FIG. 2B) and MCF (FIG. 2C) and a second reduction at around 16-32 μg/mL. We determined the MPC for both MCF and CMP1 to be 16 μg/mL against C. albicans and C. glabrata.

Selection of resistance strains in vivo predominantly occurs over a range of drug concentrations falling between the MIC and MPC values. Dosing paradigms that result in plasma drug concentrations in excess of the MPC are therefore more desirable and effective in preventing the development of de novo mutants during the course of therapy (see, e.g., Drlica et al., J. Antimicrob. Chemother. 52:11-17, 2003). Currently approved treatment regimens for caspofungin, micafungin, and anidulafungin involve once-daily dosing at levels such that the Cₘₐₓ is unlikely to be equivalent to or exceed the MPC at any point during treatment. The MPC determined for CMP1 vs. C. albicans and C. glabrata was 16 μg/mL. Modeling of CMP1 total plasma concentrations based on pharmacokinetic data allowed us to calculate that an IV administration of CMP1 50 mg would be sufficient to generate concentrations in excess of 16 μg/mL. CMP1 has the potential to be dosed at levels exceeding the MPC, and thus have a stronger mutant prevention capacity than existing approved echinocandin treatment regimens.

### Example 3

### Determination of Compound 1 Spontaneous Mutation Frequencies and Underlying Resistance Mechanisms in Candida spp.

The experiment investigated the frequency of and genetic basis for spontaneous, single-step mutations in Candida spp.

Reagents. Compound 1 (CMP1), caspofungin (CAS), anidulafungin (ANID), and amphotericin B (AMB) stocks were prepared freshly in 100% DMSO prior to use in MIC assays and spontaneous resistance experiments.

Strains. Representative wild-type strains of C. albicans (NRRL Y-477), C. glabrata (ATCC 2001, ATCC 90030), C. parapsilosis (CP02), and C. krusei (ATCC 6258) were chosen following prescreening on Sabouraud dextrose agar (SDA) plates containing each echinocandin to ensure that they had non-paradoxical agar growth phenotypes.

Antifungal susceptibility testing. Candida strains were cultured aerobically at 35° C. on SDA plates or in RPMI 1640 broth (pH 7.0). MIC assays were performed via broth microdilution in accordance with Clinical and Laboratory Standards Institute (CLSI) with the exception that test compounds were made up at 50× final assay concentration (2 μL added to 98 μL of broth containing cells at 0.5-2.5×10³ CFU/mL). MIC plates were read following a 24-hour incubation at 35° C. and MIC values were reported as concentrations resulting in prominent growth inhibition (˜50%) per CLSI guidance for echinocandins. MIC assays were performed in triplicate.

Spontaneous mutant selection. Large-format 245×245 mm assay dishes (Corning cat. no. 431272) were prepared with 150 mL SDA containing CMP1, ANID, or CAS at the minimum concentration required to cleanly inhibit growth for each Candida strain. Three individual colonies from each strain were used to start cultures in RPMI. When cultures reached ˜1.0 OD₅₃₀ they were pelleted and resuspended in phosphate-buffered saline to a cell density of −1×10⁸ CFU/mL. One milliliter aliquots were spread onto SDA plates containing drug with sterile glass beads. Starting viable count was enumerated by triplicate plating of serial dilutions of the starting inoculum. Plates were incubated at 35° C. for 48 hours. Glycerol stocks of putative mutant colonies were stored at −80° C. Mutant resistance phenotypes were confirmed by subculturing on SDA plates containing an equivalent amount of drug as was used in their initial selection. Spontaneous mutation frequencies were calculated by dividing the number of resistant colonies on a given plate by the starting inoculum plated. A subset of mutants (weighted heavily towards those selected with CMP1) were then evaluated by MIC and sequencing of FKS gene hot spot regions.

Sequence analysis of FKS gene hot spot regions. FKS1 hot spot 1 (HS1) and hot spot 2 (HS2) regions were amplified by PCR as described in Garcia-Effron et al., Antimicrobial Agents Chemother. 55:2245-2255, 2011. For C. glabrata strains, FKS2 HS1 and HS2 regions were also amplified. PCR products were sequenced using upstream forward primers for each HS region and analyzed alongside wild-type FKS sequences using Vector NTI®software (Life Technologies).

Agar drug concentrations required to provide complete background growth inhibition were between 1× and 4× the corresponding broth microdilution values.

TABLE 6

| | Broth MIC (μg/mL) | | | Plate concentration (μg/mL) | | |
|---|---|---|---|---|---|---|
| Strain | CMP1 | ANID | CAS | CMP1 | ANID | CAS |
| C. albicans | 0.03 | 0.015 | 0.25 | 0.06 | 0.015 | 0.25 |
| NRRL Y-477 | | | | | | |
| C. glabrata | 0.06 | 0.06 | 0.25 | 0.25 | 0.125 | 0.5 |
| ATCC 90030 | | | | | | |
| C. glabrata | 0.125 | 0.06 | 0.25 | 0.25 | 0.125 | 0.5 |
| ATCC 2001 | | | | | | |
| C. parapsilosis | 2 | 4 | 0.5 | 8 | 16 | 1 |
| CP02 | | | | | | |
| C. krusei | 0.06 | 0.06 | 0.5 | 0.25 | 0.25 | 0.5 |
| ATCC 6258 | | | | | | |

Tables 7a and 7b show the replicate and median mutation frequencies in Candida spp. A total of 472 spontaneous mutants were recovered following selection of CMP1, ANID, and CAS vs. 5 Candida strains (63 for CMP1, 128 for ANID, and 281 for CAS). Spontaneous mutation frequencies were lower for C. krusei and C. parapsilosis than for C. albicans and C. glabrata. CMP1 median mutation frequencies ranged from 5.0×10⁻⁸ to 3.86×10⁻⁹, comparable to those for CAS and ANID.

TABLE 7a
Replicate mutation frequencies

| | | CMP1 | | ANID | | CAS | |
|---|---|---|---|---|---|---|---|
| Strain | Plate # | Col. # | Freq. | Col. # | Freq. | Col. # | Freq. |
| C. albicans | 1 | 4 | 5.00E−08 | 15 | 1.88E−07 | 1 | 1.25E−08 |
| NRRL Y-477 | 2 | 2 | 2.27E−08 | 14 | 1.59E−07 | 1 | 1.14E−08 |
| | 3 | 7 | 5.47E−08 | 20 | 1.56E−07 | 1 | 7.81E−09 |
| C. glabrata | 1 | 3 | 1.35E−08 | 2 | 9.01E−09 | 57 | 2.57E−07 |
| ATCC 90030 | 2 | 8 | 3.52E−08 | 1 | 4.41E−09 | 110 | 4.85E−07 |
| | 3 | 3 | 1.26E−08 | 5 | 2.10E−08 | 82 | 3.45E−07 |
| C. glabrata | 1 | 9 | 3.16E−08 | 20 | 7.02E−08 | 9 | 3.16E−08 |
| ATCC 2001 | 2 | 13 | 3.79E−08 | 23 | 6.71E−08 | 10 | 2.92E−08 |
| | 3 | 3 | 1.17E−08 | 28 | 1.09E−07 | 9 | 3.50E−08 |
| C. parapsilosis | 1 | 2 | 2.08E−08 | 0 | <1.04E−08 | 0 | <1.04E−08 |
| CP02 | 2 | 1 | 9.62E−09 | 0 | <9.62E−09 | 0 | <9.62E−09 |
| | 3 | 2 | 2.08E−08 | 0 | <1.04E−08 | 0 | <1.04E−08 |
| C. krusei | 1 | 4 | 1.54E−08 | 0 | <3.38E−09 | 0 | <3.86E−09 |
| ATCC 6258 | 2 | 1 | 3.51E−09 | 0 | <3.51E−09 | 1 | 3.51E−09 |
| | 3 | 1 | 3.86E−09 | 0 | <3.38E−09 | 0 | <3.86E−09 |

TABLE 7b
Media mutation frequencies

| Strain | CMP1 | ANID | CAS |
|---|---|---|---|
| C. albicans NRRL Y-477 | 5.00E−08 | 1.59E−07 | 1.14E−08 |
| C. glabrata ATCC 90030 | 1.35E−08 | 9.01E−09 | 3.45E−07 |
| C. glabrata ATCC 2001 | 3.16E−08 | 7.02E−08 | 3.16E−08 |
| C. parapsilosis CP02 | 2.08E−08 | <1.04E−08 | <1.04E−08 |
| C. krusei ATCC 6258 | 3.86E−09 | <3.38E−09 | <3.86E−09 |

Additionally seven different fks hot spot mutations were identified in 19 strains which typically had the most shifted MIC values. fks mutations were identified in residues with clinical precedence for echinocandin resistance. Echinocandin cross-resistance was observed for all mutants. Table 8 shows a summary of mutants possessing fks mutations.

TABLE 8

| | MIC (μg/mL) | | | | Fks amino acid substitutions | | | |
|---|---|---|---|---|---|---|---|---|
| Strain | CMP1 | ANID | CAS | AMB | Fks1 HS1 | Fks1 HS2 | Fks2 HS1 | Fks2 HS2 |
| C. albicans | 0.03 | 0.015 | 0.25 | 0.5 | WT | WT | WT | WT |
| NRRL Y-477 | 0.25 | 0.25 | 0.5 | 0.25 | S645P† | WT | NS | NS |
| C. glabrata | 0.06 | 0.06 | 0.25 | 0.5 | WT | WT | WT | WT |
| ATCC 90030 | 2 | 2 | 4 | 0.5 | WT | WT | ΔF659 | WT |
| | 0.25 | 1 | 1 | 0.5 | WT | WT | D666H | WT |
| C. glabrata | 0.125 | 0.06 | 0.25 | 0.5 | WT | WT | WT | WT |
| ATCC 2001 | 0.25 | 0.25 | 0.25 | 0.25 | WT | WT | WT | R1378S |
| | 2 | 4 | 16 | 0.25 | WT | WT | ΔF659 | WT |
| | 0.5 | 0.5 | 0.5 | 0.5 | WT | WT | D666Y | WT |
| | 1 | 1 | 1 | 0.25 | WT | WT | S663F | WT |
| | 0.5 | 0.5 | 0.5 | 0.25 | WT | WT | R665G | WT |
| | 0.5 | 0.5 | 0.25 | 0.5 | WT | WT | D666N | WT |

This experiment shows that median spontaneous mutation frequencies for CMP1 among the Candida spp. tested were low, ranging from 5.00×10⁻⁸ to 3.86×10⁻⁹. These values were within the ranges generated for ANID and CAS. Of the 4 Candida spp. evaluated, the C. glabrata strains had the highest mutation frequencies and MIC fold-shift increases for all three drugs. All CMP1-selected mutants with MIC values increasing 2-fold also demonstrated cross-resistance to ANID and/or CAS. Eight different fks HS mutations (Fks1 HS1: S645P; Fks2 HS1: ΔF659, S663F, R665G, D666H, D666Y, D666N; Fks2 HS2: R1378S) were identified among 25 strains out of the 82 sequenced, and were typically found in mutants with the largest MIC shifts for all three drugs. Consistent with clinical trends, the C. glabrata strains had the highest resistance incidences of all the Candida spp. evaluated. Many of the “spontaneous mutant” colonies selected at 1× the agar inhibition level demonstrated insignificant changes in MIC values (i.e., 2-fold) suggesting that spontaneous mutations conferring >2-fold MIC shifts to all 3 echinocandins are even less frequent than the values derived in these plating experiments.

Single-step mutations conferring reduced susceptibility to CMP1 in Candida spp. are rare. Because of CMP1's unique front-loading dosing paradigm and extended half-life, the higher achievable Cₘₐₓ and AUC values may help prevent the emergence of spontaneous resistance during the course of therapy.

### Example 4

### Characterization of Resistance Following Serial Passage of Candida spp. in the Presence of Compound 1

The purpose of this experiment was to investigate the potential for clinically relevant Candida spp. to develop reduced susceptibility to Compound 1 (CMP1) following serial exposure, genetically characterize the underlying resistance mechanisms, and compare cross-resistance trends with anidulafungin and caspofungin.

Reagents. Compound 1 (CMP1), caspofungin (CAS), anidulafungin (ANID), and amphotericin B (AMB) stocks were prepared freshly in 100% DMSO prior to use in MIC assays and serial passage experiments.

Strains. Representative wild-type strains of C. albicans (NRRL Y-477), C. glabrata (ATCC 2001, ATCC 90030), C. parapsilosis (CP02), and C. krusei (ATCC 6258) were chosen following prescreening on Sabouraud dextrose agar (SDA) plates containing each echinocandin to ensure that they had clean, non-paradoxical agar growth phenotypes.

Antifungal susceptibility testing. Candida strains were cultured aerobically at 35° C. on SDA plates or in RPMI 1640 broth (pH 7.0). MIC assays were performed via broth microdilution in accordance with Clinical and Laboratory Standards Institute (CLSI) with the exception that test compounds were made up at 50× final assay concentration (2 μL added to 98 μL of broth containing cells at 0.5-2.5×10³ CFU/mL). MIC plates were read following a 24-hour incubation at 35° C. and MIC values are reported as concentrations resulting in prominent growth inhibition (˜50%) per CLSI guidance for echinocandins. MIC assays were performed in triplicate.

Serial passage. SDA drug gradient plates were created by pouring two overlapping layers of media as described in Bryson et al., Science 116:45-51, 1952, using 90×90 mm square petri dishes. As reduced susceptibility developed, drug concentrations were increased to maintain the leading edge of growth within the drug gradient. Following each passage the leading edge of growth (i.e., most resistant cells) was resuspended in 0.85% NaCl to an absorbance of ˜1.0 OD₅₃₀ and a 100 μL aliquot (˜1.0×10⁶ CFU) was spread onto a fresh passage plate using sterile glass beads. As strains developed reduced susceptibility to selecting drugs and were able to grow past the halfway point on the gradient plate, drug concentrations were increased 2-fold for subsequent passages. A glycerol stock was made from the total cell population for each culture condition for each passage. Twenty serial passages were completed for each drug/strain combination. MIC testing was performed on total cell populations for each group every 5th passage.

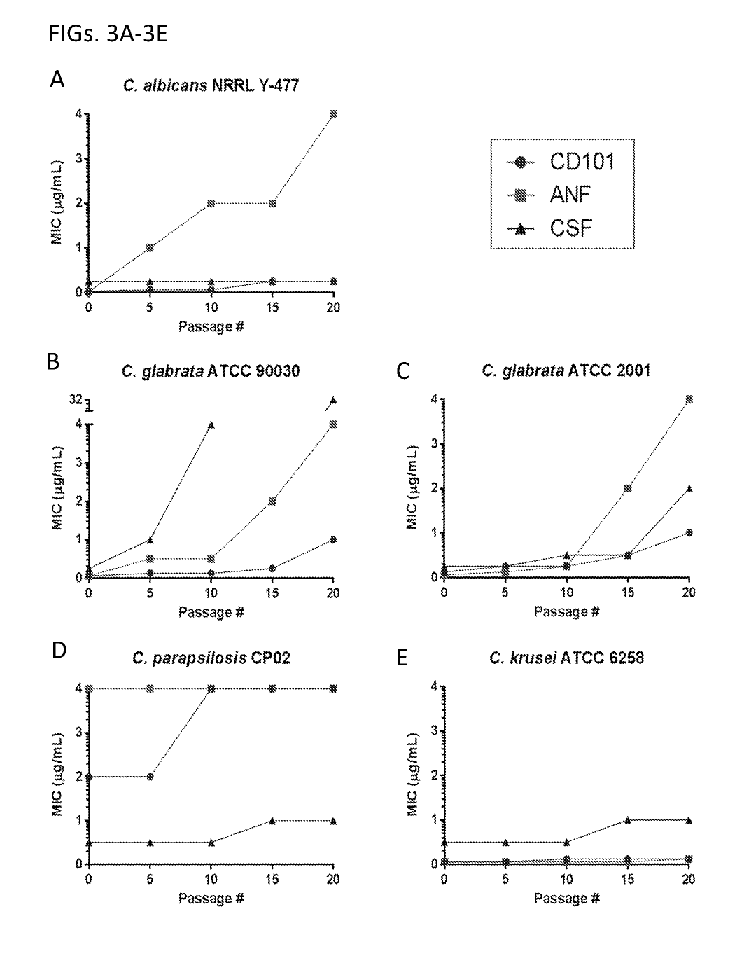

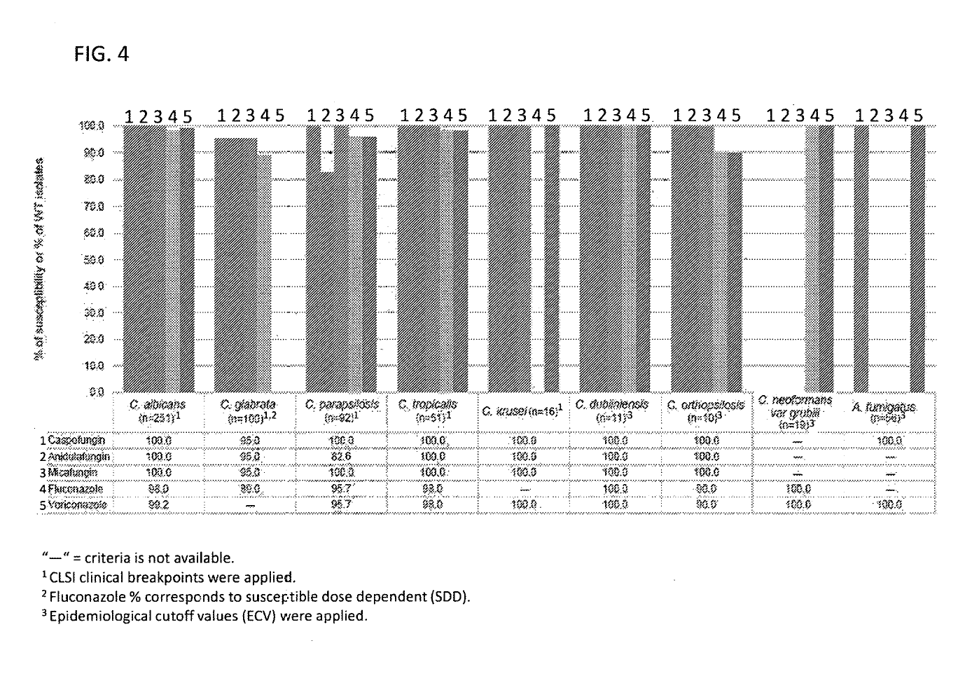

Analysis of individual passage 20 colonies. Passage #20 (P20) total populations were streaked to isolation on SDA and three colonies were selected. All three colonies were assessed via MIC, and a representative colony of the total population MIC was selected for further analysis. Sequence analysis of FKS gene hot spot regions. FKS1 hot spot 1 (HS1) and hot spot 2 (HS2) regions were amplified by PCR as described in Garcia-Effron et al., Antimicrobial Agents Chemother. 55:2245-2255, 2011. For C. glabrata strains, FKS2 HS1 and HS2 regions were also amplified. PCR products were sequenced using upstream forward primers for each HS region and analyzed alongside WT FKS sequences using Vector NTI® software (Life Technologies). FIGS. 3A, 3B, 3C, 3D, and 3E show that reduced susceptibility was observed for CMP1, ANID, and CAS vs. most of the 5 Candida strains following 20 serial passages. C. glabrata strains (FIGS. 4B and 4C) had the most consistently high MIC shifts at P20 for all drugs, followed by C. albicans (FIG. 3A), and finally C. parapsilosis (FIG. 3D) and C. krusei (FIG. 3E) had the lowest resistance potential of all strains tested. Table 9 shows the MIC values and MIC fold-shifts of CMP1, ANID, and CAS against Candida spp. from wild-type strain to the strain at P20. Serial passage generated strains that were CAS-resistant for both C. glabrata strains and C. krusei and ANID-resistant for both C. glabrata strains and for C. albicans following 20 passages.

TABLE 9
MIC fold shift increases over 20 serial passages

| | | MIC (μg/mL) | | |
|---|---|---|---|---|
| Strain | Selecting drug | WT | P20 | MIC fold-shift |
| C. albicans | CMP1 | 0.03 | 0.25* | 8 |
| NRRL Y-477 | ANID | 0.015 | 4*^{‡} | 256 |
| | CAS | 0.25 | 0.25 | 1 |
| C. glabrata | CMP1 | 0.06 | 1 | 16 |
| ATCC 90030 | ANID | 0.06 | 4*^{‡} | 64 |
| | CAS | 0.25 | 32*^{‡} | 128 |
| C. glabrata | CMP1 | 0.125 | 1 | 8 |
| ATCC 2001 | ANID | 0.06 | 4^{‡} | 64 |
| | CAS | 0.25 | 2^{‡} | 8 |
| C. parapsilosis | CMP1 | 2 | 4 | 2 |
| CP02 | ANID | 4 | 4 | 1 |
| | CAS | 0.5 | 1 | 2 |
| C. krusei | CMP1 | 0.06 | 0.125 | 2 |
| ATCC 6258 | ANID | 0.06 | 0.125 | 2 |
| | CAS | 0.5 | 1^{‡} | 2 |

*required 48 hours of incubation to read MIC values;
† homozygous mutation;
WT, wild-type; HS1, hot spot 1; HS2, hot spot 2; NS, not sequenced.

Additionally, MIC and FKS gene hot spot sequence analyses of individual P20 strains were performed (Table 10). Cross-resistance with at least one other drug was observed for all P20 strains with any level of reduced susceptibility to the selecting drug. With the exception of C. krusei, only strains with highly shifted MIC values possessed mutations in FKS hot spot regions. Fks residues mutated at hotspot positions S645 (C. albicans), D632, D666, and F659 (C. glabrata) have clinical precedence while the substitution at 11366 in P20 C. krusei has not been observed.

TABLE 10

| | | | | | | | Fks amino acid changes | | | |
|---|---|---|---|---|---|---|---|---|---|---|
| | Selecting | | MIC (μg/mL) | | | | Fks1 | Fks1 | Fks2 | Fks2 |
| Strain | drug | Strain | CMP1 | ANID | CAS | AMB | HS1 | HS2 | HS1 | HS2 |
| C. albicans | — | WT | 0.03 | 0.015 | 0.25 | 0.5 | WT | WT | WT | WT |
| NRRL | CMP1 | P20-1* | 0.25 | 0.125 | 0.5 | 0.5 | WT | WT | NS | NS |
| Y-477 | ANID | P20-1* | 2 | 4 | 2 | 0.5 | S645Y^{†} | WT | NS | NS |
| | CAS | P20-1 | 0.03 | 0.015 | 0.25 | 0.25 | WT | WT | NS | NS |
| C. glabrata | — | WT | 0.06 | 0.06 | 0.25 | 0.5 | WT | WT | WT | WT |
| ATCC | CMP1 | P20-2 | 1 | 1 | 1 | 0.5 | WT | WT | WT | WT |
| 90030 | ANID | P20-1* | 2 | 4 | 2 | 0.5 | WT | WT | D666I | WT |
| | CAS | P20-1* | 8 | 8 | 32 | 0.25 | WT | WT | F659I | WT |
| | | | | | | | | | D666Y | |
| C. glabrata | — | WT | 0.125 | 0.06 | 0.25 | 0.5 | WT | WT | WT | WT |
| ATCC | CMP1 | P20-2 | 1 | 1 | 1 | 0.25 | WT | WT | WT | WT |
| 2001 | ANID | P20-2 | 2 | 4 | 2 | 1 | D632Y | WT | WT | WT |
| | CAS | P20-2 | 2 | 2 | 2 | 0.5 | WT | WT | WT | WT |
| C. parapsilosis | — | WT | 2 | 4 | 0.5 | 0.5 | WT | WT | WT | WT |
| CP02 | CMP1 | P20-1 | 4 | 8 | 1 | 0.5 | WT | WT | NS | NS |
| | ANID | P20-1 | 4 | 4 | 0.5 | 0.5 | WT | WT | NS | NS |
| | CAS | P20-1 | 2 | 4 | 1 | 0.5 | WT | WT | NS | NS |
| C. krusei | — | WT | 0.06 | 0.06 | 0.5 | 1 | WT | WT | WT | WT |
| ATCC | CMP1 | P20-2 | 0.125 | 0.25 | 1 | 1 | WT | I1366S^{†} | NS | NS |
| 6258 | ANID | P20-2 | 0.125 | 0.125 | 0.5 | 1 | WT | WT | NS | NS |
| | CAS | P20-2 | 0.25 | 0.25 | 1 | 1 | WT | I1366S^{†} | NS | NS |

*required 48 hours of incubation to read MIC values;
^{†}homozygous mutation;
WT, wild-type;
HS1, hot spot 1;
HS2, hot spot 2;
NS, not sequenced.

This experiment shows that CMP1 had the smallest overall MIC fold-shift increases at passage #20. The potential for resistance development vs. CMP1 among 4 key clinically-relevant Candida species was low. Maximal P20 CMP1 MIC values for CMP1-selected strains were 0.25 μg/mL for C. albicans, 1 μg/mL for both C. glabrata strains, 4 μg/mL for C. parapsilosis, and 0.125 μg/mL for C. krusei, corresponding to MIC fold shift increases equivalent to or lower than the majority of those generated under selection with ANID and CAS. Cross-resistance was broadly observed among CMP1, ANID, and CAS evaluated and there were no CMP1-selected mutants that conferred reduced susceptibility to CMP1 but not also to ANID and/or CAS. Of the 15 selection groups, 6 of them had P20 strains possessing a total of 5 different fks HS mutations (Fks1 HS1: S645Y, D632Y; Fks1 HS2: I1366S; Fks2 HS1: D6661, F6591/D666Y), all of which were homozygous. With the exception of C. krusei, only P20 strains with the largest MIC shifts possessed fks HS mutations. Consistent with clinical observations and its haploid nature, C. glabrata demonstrated the highest potential for echinocandin resistance development.

### Example 5

### Evaluation of Compound 1 With Echinocandin and Azole Comparators Against Candida spp. Isolated From Patients With Vulvovaginal Candidiasis (VVC)

Fungicidal activity against Candida spp. makes echinocandins appealing for treatment of VVC, particularly refractory or azole-resistant infections. However, the instability and infusion delivery of approved agents (MCF, CAS, and ANID) are barriers to their utilization. Unlike currently marketed echinocandins (MCF, CAS, and ANID), Compound 1 (CMP1) has stability suitable for topical formulations. Over 90% of azole-resistant and recurrent VVC are caused by C. albicans or C. glabrata. Based on observations from systemic Candida infections, the echinocandins should have many advantages over azoles for the treatment of VVC: cidal vs. static, less development of resistance, fewer drug interactions, and better overall safety. In this experiment, we evaluated Compound 1 (CMP1), echinocandins such as caspofungin (CAS), micafungin (MCF), and anidulafungin (ANID), and therapeutic azoles such as fluconazole (FLU) and itraconazole (ITR) against VVC isolates in vitro at a vaginal pH to assess whether CMP1 has sufficient potency to treat VVC, including infections caused by azole-resistant pathogens.

Vaginal isolates of Candida spp. were obtained from the Wayne State Vaginitis Clinic organism bank and were comprised of C. albicans (60 total, 10 fluconazole-resistant), C. glabrata (21 total, 11 fluconazole-resistant), C. parapsilosis (14 total, 7 fluconazole-resistant), and C. tropicalis (14). Isolates were plated on CHROMagar to verify purity; plates were incubated for 48 h at 37° C. A single colony was resubcultured on Sabouraud Dextrose agar and incubated for 24 h at 35° C. Susceptibility testing was performed at pH 7 and 4 using the broth microdilution method described in CLSI document M27-A3. A MOPS (morpholinepropane-sulfonic acid) buffer solution was used for pH adjustment. Fluconazole was tested at a range of 0.125-64 μg/mL; all other antifungal agents were tested at 0.008-4 μg/mL. A yeast inoculum (approx 1.5×10³ CFU/mL) in RPMI 1640 medium was added to each well, and trays were incubated for 24 h and 48 h at 35° C. MICs were read as the lowest antifungal concentration with 80% growth reduction compared to growth in the antifungal-free growth well for all test articles.

Table 11 a shows the MIC90 values for CMP1 and comparators against 108 VVC clinical isolates at pH 7/4 read at 24 h. Table 11b shows the MIC90 values for CMP1 and comparators against 108 VVC clinical isolates at pH 7/4 read at 48 h. From 24 to 48 h, MICs shifted upward 2- to 4-fold for each of CMP1, CAS, MCF, and ANID and at least that much for the azoles. Multiple isolates from C. albicans, C. glabrata, and C. tropicalis had MICs>4 μg/mL for the two azoles, but none had MICs>4 μg/mL for CMP1, CAS, MCF, and ANID.

TABLE 11a

| | MIC₉₀ (μg/mL) at pH 7/4 read at 24 h | | | |
|---|---|---|---|---|
| | C. albicans | C. glabrata | C. parapsilosis | C. tropicalis |
| | (60) | (21) | (14) | (13) |
| CMP1 | 0.06/0.25 | 0.125/0.5 | 2/2 | 0.125/0.5 |
| FLU | 8/8 | 16/64 | i.g.*/8 | 2/8 |
| ITR | 0.06/0.125 | 1/1 | 0.03/0.016 | 0.125/0.125 |
| CAS | 0.5/0.5 | 1/1 | 1/1 | 1/1 |
| MCF | 0.008/0.125 | 0.008/0.016 | 1/0.5 | 0.016/0.06 |
| ANID | 0.008/0.03 | 0.03/0.125 | 2/1 | 0.03/0.125 |

*i.g. = Insufficient growth. Only 7 of the 14 organisms exhibited sufficient growth to obtain MIC values. The range was from 0.125 to 16 μg/mL.

TABLE 11b

| | MIC₉₀ (μg/mL) at pH 7/4 read at 48 h | | | |
|---|---|---|---|---|
| | C. albicans | C. glabrata | C. parapsilosis | C. tropicalis |
| | (60) | (21) | (14) | (13) |
| CMP1 | 0.125/0.5 | 0.25/1 | 4/2 | 0.125/2 |
| FLU | 16/>64 | >64/>64 | 32/>64 | 64/>64 |
| ITR | 0.125/05 | >4/>4 | 0.125/0.125 | 0.5/0.5 |
| CAS | 1/1 | 2/2 | 2/4 | 2/2 |
| MCF | 0.03/0.25 | 0.016/0.03 | 2/1 | 0.03/0.06 |
| ANID | 0.03/0.06 | 0.06/0.25 | 4/2 | 0.06/0.5 |

Table 12 shows MIC values for CMP1 and comparators against 10 VVC clinical isolates of fluconazole-resistant C. albicans at pH 7/4 read at 48 h.

TABLE 12

| | MIC (μg/mL) at pH 7/4 at 48 h | | | | | |
|---|---|---|---|---|---|---|
| Isolate | CMP1 | FLU | ITR | CAS | MCF | ANID |
| 1 | 0.03/0.5 | 16/>64 | 0.125/0.125 | 0.5/0.25 | 0.008/0.016 | 0.008/0.03 |
| 2 | 0.25/0.125 | 16/32 | 0.25/0.25 | 0.5/1.0 | 0.016/0.016 | 0.03/0.03 |
| 3 | 0.03/0.5 | 16/>64 | 0.5/0.5 | 0.5/0.5 | 0.008/0.016 | 0.008/0.03 |
| 4 | 0.06/0.25 | 8/>64 | 0.03/0.125 | 0.5/1.0 | 0.008/0.03 | 0.008/0.06 |
| 5 | 0.125/0.25 | >64/>64 | 0.25/>4 | 2/4 | 0.06/0.016 | 0.016/0.125 |
| 6 | 0.03/0.5 | 0.25/>64 | 0.016/>4 | 1/1 | 0.008/0.03 | 0.008/0.06 |
| 7 | 0.03/0.5 | 16/32 | 0.5/0.5 | 0.5/1 | 0.06/0.25 | 0.008/0.06 |
| 8 | 0.125/0.25 | 32/>64 | 2/>4 | 0.5/0.5 | 0.008/0.06 | 0.008/0.06 |
| 9 | 1/1 | >64/>64 | >4/>4 | 2/1 | 0.06/0.125 | 0.125/0.25 |
| 10 | 0.125/0.25 | 16/64 | 0.125/0.25 | 0.5/1 | 0.008/0.06 | 0.008/0.06 |

Additionally, Table 13 shows the distribution of MIC values for CMP1 and approved azole antifungals against 60 C. albicans and 21 C. glabrata VVC clinical isolates at pH 7 and 4 read at 24 and 48 h.

TABLE 13

| | | MIC (μg/mL) | | | | | | | | | | |
|---|---|---|---|---|---|---|---|---|---|---|---|---|
| | | 0.008 | 0.016 | 0.03 | 0.06 | 0.125 | 0.25 | 0.5 | 1 | 2 | 4 | ≥4 |
| C. albicans 24 h | | | | | | | | | | | | |
| CMP1 | pH = 7 | | 5 | 43 | 10 | 2 | | | | | | |
| | pH = 4 | | | | 1 | 33 | 23 | 3 | | | | |
| FLU | pH = 7 | | | | | 25 | 19 | 7 | 1 | 2 | | 6 |
| | pH = 4 | | | | | | 35 | 13 | 2 | 2 | 1 | 7 |
| ITR | pH = 7 | 21 | 20 | 9 | 3 | | 2 | 2 | | 1 | | |
| | pH = 4 | 8 | 24 | 13 | 8 | 2 | 2 | 2 | | 1 | | |
| C. albicans 48 h | | | | | | | | | | | | |
| CMP1 | pH = 7 | | 2 | 29 | 16 | 9 | 1 | 1 | 2 | | | |
| | pH = 4 | | | | | 6 | 16 | 34 | 4 | | | |
| FLU | pH = 7 | | | | | 3 | 37 | 8 | 2 | 1 | | 9 |
| | pH = 4 | | | | | | | 27 | 13 | 5 | 1 | 14 |
| ITR | pH = 7 | 6 | 34 | 8 | | 6 | 2 | 2 | | 1 | | 1 |
| | pH = 4 | | 10 | 23 | 10 | 6 | 3 | 3 | 1 | | | 4 |
| C. glabrata 24 h | | | | | | | | | | | | |
| CMP1 | pH = 7 | | | | 3 | 17 | | | | 1 | | |
| | pH = 4 | | | | | 2 | 3 | 15 | | 1 | | |
| FLU | pH = 7 | | | | | | | 4 | 2 | 5 | 2 | 8 |
| | pH = 4 | | | | | | | | | 5 | 3 | 13 |
| ITR | pH = 7 | | | 4 | 2 | 6 | 3 | 3 | 2 | | 1 | |
| | pH = 4 | | | 1 | 2 | | 2 | 9 | 6 | 1 | | |
| C. glabrata 48 h | | | | | | | | | | | | |
| CMP1 | pH = 7 | | | | 2 | 16 | 1 | 1 | | 1 | | |
| | pH = 4 | | | | | | 2 | 8 | 9 | 2 | | |
| FLU | pH = 7 | | | | | | | | 3 | 4 | 2 | 12 |
| | pH = 4 | | | | | | | | | 1 | 2 | 18 |
| ITR | pH = 7 | | | 1 | 1 | 1 | 5 | 8 | | 2 | | 3 |
| | pH = 4 | | | 1 | 1 | 1 | | 2 | 4 | 2 | 2 | 8 |

This experiment shows that VVC isolates resistant to FLU and ITR were not cross-resistant to CMP1, CAS, MCF, and ANID. CMP1, CAS, MCF, and ANID showed little MIC shift as a function of pH. CAS generally had the highest MIC values; MCF had the lowest. CMP1 has sufficient potency and stability at low pH to be evaluated in cream and gel formulations as a potential topical treatment for VVC, including refractory and azole-resistant infections.

### Example 6

### Activity of Compound 1 and Comparator Antifungal Agents Tested Against Contemporary Invasive Fungal Isolates

In this experiment, we determined the activity and potency of Compound 1 (CMP1) and comparator antifungal agents tested against 606 clinical fungal isolates collected worldwide from invasive fungal infections.

Fungal organisms. A total of 606 non-duplicate prospectively collected fungal isolates from 38 medical centers located in North America (161 isolates; 10 sites), Europe (294; 17), the Asia-Pacific Region (82; 6) and Latin America (69; 5) were evaluated. Isolates selected were from the following sources: bloodstream, (379 strains), normally sterile body fluids, tissues or abscesses (22 strains), respiratory tract specimens (96 strains) and 109 were collected from other or non-specified body sites.

Species identification. Yeast isolates were subcultured and screened using CHROMagar Candida (Becton Dickinson, Sparks, Md. USA) to ensure purity and to differentiate Candida albicans/Candida dubliniensis, Candida tropicalis and Candida krusei. Isolates suspected to be either C. albicansor C. dubliniensis (green colonies on CHROMagar) were incubated at 45° C. All other yeast isolates were submitted to Matrix-Assisted Laser Desorption Ionization-Time of Flight Mass Spectrometry (MALDI-TOF MS) using the MALDI Biotyper according to the manufacturer's instructions (Bruker Daltonics, Billerica, Mass. USA). Isolates that were not identified by either phenotypic or proteomic methods were identified using sequencing-based methods as previously described.

Antifungal susceptibility testing. All isolates were tested by broth microdilution according to Clinical and Laboratory Standards Institute (CLSI) methods outlined in documents M27-A3 and M38-A2. Frozen-form panels used RPMI 1640 broth supplemented with MOPS (morpholinepropanesulfonic acid) buffer and 0.2% glucose and inoculated with 0.5 to 2.5×10³ CFU/mL suspensions. MIC/MEC values were determined visually, after 24, 48 or 72 hours of incubation at 35° C., as the lowest concentration of drug that resulted in ≥50% inhibition of growth relative to the growth control or complete (100%) inhibition. CLSI clinical breakpoints were used for the five most common species of Candida (C. albicans, C. glabrata, C. parapsilosis, C. tropicalis, and C. krusei) for echinocandins, fluconazole and voriconazole. Epidemiological cutoff values (ECV) were applied when available.

Quality control was performed as recommended in CLSI documents M27-A3 and M38-A2 using strains C. krusei ATCC 6258, C. parapsilosis ATCC 22019, A. flavus ATCC 204304 and A. fumigatus MYA-3626.

As shown in Table 14, Compound 1 (CMP1) (MIC_{50/90}, 0.03/0.06 μg/mL) inhibited all 251 C. albicans isolates at ≤0.12 μg/mL. CMP1 displayed activity most similar to that of caspofungin (CAS) (MIC_{50/90}, 0.03/0.06 μg/mL). CMP1 inhibited 95 (95.0%) of the C. glabrata isolates at ≤0.12 μg/mL, the activity of which was two-fold greater when compared to anidulafungin (ANID) or CAS (MIC₅₀ and MIC₉₀, 0.06 and 0.12 μg/mL for both compounds) and two-fold less than the activity of micafungin (MCF) (MIC₅₀ and MIC₉₀, 0.015 and 0.03 μg/mL). Moreover, all C. parapsilosis isolates were inhibited by CMP1 (MIC₅₀ and MIC₉₀, 1 and 2 μg/mL) at pg/mL. CMP1 displayed similar activity to that of MCF (MIC50/90, 1/2 μg/mL), slightly greater activity when compared to ANID (MIC_{50/90}, 2/4 μg/mL) and was two-fold less active than CAS (MIC_{50/90}, 0.5/1 μg/mL). C. tropicalis isolates (n=51) were considered susceptible to MCF, CAS, ANID, and CMP1 (MIC_{50/90}, 0.015/0.06 μg/mL) inhibited all isolates at ≤0.06 μg/mL. CMP1 (MIC₅₀ and MIC₉₀, 0.03 and 0.06 μg/mL) was very active against 16 C. krusei and all isolates were inhibited at ≤0.06 μg/mL. The activity of CMP1 (MIC₅₀ and MIC₉₀, 0.03 and 0.06 μg/mL) against C. dubliniensis isolates was comparable to that of CAS (MIC₅₀ and MIC₉₀, 0.03 and 0.06 pg/mL). CMP1 (MIC₅₀ and MIC₉₀, 0.5 and 1 μg/mL) activity against C. orthopsilosis was similar to the activity of ANID and MCF (MIC_{50/90}, 0.5/1 μg/mL for both). CAS was two-fold more active against C. orthopsilosis isolates (MIC₅₀ and MIC₉₀, 0.25 and 0.5 μg/mL) when compared to others. MCF, CAS, ANID, and CMP1 had limited activity against C. neoformans var. grubii isolates (n=19); all isolates had MIC values at μg/mL for these compounds. MCF, CAS, ANID, and CMP1 displayed good activity against A. fumigatus; CMP1 (MEC₅₀ and MEC₉₀, 0.015 and 0.015 μg/mL) activity was two-fold greater than that of CAS (MEC50/90, 0.03/0.03 μg/mL) and similar to that of MCF. ANID (MEC_{50/90}, 0.008/0.015 μg/mL) was slightly more active than the other compounds from the same class.

TABLE 14
Antifungal activity of Compound 1 (CMP1), anidulafungin (ANID), caspofungin (CAS), and
micafungin (MCF) against organisms/organism groups tested using the CLSI reference method

| Organism | | | | | | | | | | | | | | |
|---|---|---|---|---|---|---|---|---|---|---|---|---|---|---|
| species/ | | | | | | | | | | | | | | |
| groups | | | | | | | | | | | | | | |
| (no. | | | | | | | | | | | | | MIC/MEC | |
| tested)/ | Number (cumulative %) of isolates inhibited at MIC/MEC (μg/mL) | | | | | | | | | | | | (μg/mL) | |
| agent | ≤0.008 | 0.015 | 0.03 | 0.06 | 0.12 | 0.25 | 0.5 | 1 | 2 | 4 | 8 | >8 | 0% | 0% |

Candida albicans (251)

| | | | | | | | |
|---|---|---|---|---|---|---|---|
| CMP1 | 17 | 107 | 91 | 28 | 8 | .03 | .06 |
| | (6.8) | (49.4) | (85.7) | (96.8) | (100.0) | | |
| ANID | 69 | 101 | 54 | 22 | 5 | .015 | .06 |
| | (27.5) | (67.7) | (89.2) | (98.0) | (100.0) | | |
| CAS | 4 (1.6) | 76 | 135 | 34 | 2 | .03 | .06 |
| | | (31.9) | (85.7) | (99.2) | (100.0) | | |
| MCF | 17 | 171 | 63 | | | .015 | .03 |
| | (6.8) | (74.9) | (100.0) | | | | |

Candida glabrata (100)

| | | | | | | | | | | | | | |
|---|---|---|---|---|---|---|---|---|---|---|---|---|---|
| CMP1 | | 3 | 59 | 29 | 4 | 0 | 0 | 1 | 3 | 1 | | .03 | .06 |
| | | (3.0) | (62.0) | (91.0) | (95.0) | (95.0) | (95.0) | (96.0) | (99.0) | (100.0) | | | |
| ANID | 1 | 0 | 11 | 62 | 21 | 0 | 0 | 0 | 3 | 2 | | .06 | .12 |
| | (1.0) | (1.0) | (12.0) | (74.0) | (95.0) | (95.0) | (95.0) | (95.0) | (98.0) | (100.0) | | | |
| CAS | | | 38 | 51 | 6 | 0 | 0 | 0 | 3 | 0 | 3 | .06 | .12 |
| | | | (38.0) | (89.0) | (95.0) | (95.0) | (95.0) | (95.0) | (97.0) | (97.0) | (100.0) | | |
| MCF | 1 | 73 | 21 | 0 | 0 | 0 | 0 | 0 | 3 | 2 | | .015 | .03 |
| | (1.0) | (74.0) | (95.0) | (95.0) | (95.0) | (95.0) | (95.0) | (95.0) | (98.0) | (100.0) | | | |

Candida parapsilosis (92)

| | | | | | | |
|---|---|---|---|---|---|---|
| CMP1 | | 22 | 35 | 34 | 1 | |
| | | (23.9) | (62.0) | (98.9) | (100.0) | |
| ANID | | 4 | 28 | 44 | 16 | |
| | | (4.3) | (34.8) | (82.6) | (100.0) | |
| CAS | 31 | 49 | 11 | 1 | | .5 |
| | (33.7) | (87.0) | (98.9) | (100.0) | | |
| MCF | | 10 | 57 | 25 | | |
| | | (10.9) | (72.8) | (100.0) | | |

Candida tropicalis (51)

| | | | | | | | |
|---|---|---|---|---|---|---|---|
| CMP1 | 2 | 27 | 16 | 6 | | .015 | .06 |
| | (3.9) | (56.9) | (88.2) | (100.0) | | | |
| ANID | 5 | 27 | 18 | 1 | | .015 | .03 |
| | (9.8) | (62.7) | (98.0) | (100.0) | | | |
| CAS | | 12 | 23 | 15 | 1 | .03 | .06 |
| | | (23.5) | (68.6) | (98.0) | (100.0) | | |
| MCF | 1 | 15 | 27 | 8 | | .03 | .06 |
| | (2.0) | (31.4) | (84.3) | (100.0) | | | |

Candida krusei (16)

| | | | | | | | |
|---|---|---|---|---|---|---|---|
| CMP1 | 3 | 11 | 2 | | | .03 | .06 |
| | (18.8) | (87.5) | (100.0) | | | | |
| ANID | 1 (6.2) | 6 | 9 | | | .06 | .06 |
| | | (43.8) | (100.0) | | | | |
| CAS | | | 4 | 10 | 2 | .12 | .25 |
| | | | (25.0) | (87.5) | (100.0) | | |
| MCF | | | 8 | 8 | | .06 | .12 |
| | | | (50.0) | (100.0) | | | |

Candida dubliniensis (11)

| | | | | | | |
|---|---|---|---|---|---|---|
| CMP1 | | 6 | 4 | 1 | .03 | .06 |
| | | (54.5) | (90.9) | (100.0) | | |
| ANID | 1 | 3 | 7 | | .06 | .06 |
| | (9.1) | (36.4) | (100.0) | | | |
| CAS | 1 | 5 | 5 | | .03 | .06 |
| | (9.1) | (54.5) | (100.0) | | | |
| MCF | 3 | 8 | | | .03 | .03 |
| | (27.3) | (100.0) | | | | |

Candida orthopsilosis (10)

| | | | | | | | | |
|---|---|---|---|---|---|---|---|---|
| CMP1 | | 1 | 3 | 1 | 4 | 1 | .5 | |
| | | (10.0) | (40.0) | (50.0) | (90.0) | (100.0) | | |
| ANID | | | 3 | 2 | 4 | 1 | .5 | |
| | | | (30.0) | (50.0) | (90.0) | (100.0) | | |
| CAS | 1 | 3 | 3 | 3 | | | .25 | .5 |
| | (10.0) | (40.0) | (70.0) | (100.0) | | | | |
| MCF | | 2 | 2 | 3 | 3 | | .5 | |
| | | (20.0) | (40.0) | (70.0) | (100.0) | | | |

Cryptococcus neoformans var. grubii (19)

| | | | | |
|---|---|---|---|---|
| CMP1 | 6 | 13 | 8 | 8 |
| | (31.6) | (100.0) | | |
| ANID | | 19 | 8 | 8 |
| | | (100.0) | | |
| CAS | 4 | 15 | 8 | 8 |
| | (21.1) | (100.0) | | |
| MCF | | 19 | 8 | 8 |
| | | (100.0) | | |

Aspergillus fumigatus (56)

| | | | | | | |
|---|---|---|---|---|---|---|
| CMP1 | 23 | 29 | 4 | | .015 | .015 |
| | (41.1) | (92.9) | (100.0) | | | |
| ANID | 30 | 21 | 5 | | 0.008 | .015 |
| | (53.6) | (91.1) | (100.0) | | | |
| CAS | | 18 | 35 | 3 | .03 | .03 |
| | | (32.1) | (94.6) | (100.0) | | |
| MCF | 16 | 38 | 2 | | .015 | .015 |
| | (28.6) | (96.4) | (100.0) | | | |

Among the five C. glabrata isolates displaying resistant MIC results for MCF, CAS, ANID, and CMP1, one harbored a mutation in fksl HS1 encoding alteration S629P and another two carried alterations in fks2 HS1 F659S or S663P. The two remaining isolates were collected from the same patient in Edmonton, Canada and both strains carried alterations in fks1HS1 S629P and fks2HS1 S663P conferring elevated CAS MIC results (>8 μg/mL) and MIC results of 2-4 μg/mL for CMP1, ANID and MCF (Table 15).

TABLE 15
Activity of Compound 1 (CMP1) compared to anidulafungin (ANID), caspofungin (CAS), and
micafungin (MCF) for Candida spp. isolates harboring FKS alterations detected in this experiment.

| | | MIC according | | | | | | | |
|---|---|---|---|---|---|---|---|---|---|
| | | to CLSI method | | | | | | | |
| State and/or | | (μg/mL): | | | | 1,3-β-D-glucan synthase mutations^{a}: | | | |
| country | Organism | CMP1 | ANF | CAS | MCF | fks1 HS1 | fks1 HS2 | fks2 HS1 | fks2 HS2 |
| Israel | C. glabrata | 1 | 2 | 2 | 2 | WT | WT | F659S | WT |
| CA, USA | C. glabrata | 2 | 2 | 8 | 2 | S629P | WT | WT | WT |
| NY, USA | C. glabrata | 2 | 2 | 2 | 2 | WT | WT | S663P | WT |
| Canada | C. glabrata | 4 | 4 | >8 | 4 | S629P | WT | S663P | WT |
| Canada | C. glabrata | 2 | 4 | >8 | 4 | S629P | WT | S663P | WT |

Additionally, the activity of comparator agents tested against organisms/organism groups is displayed in FIG. 4. Fluconazole resistance was noted among 2.0% of C. albicans and C. tropicalis, 11.0% of C. glabrata and 4.3% of C. parapsilosis. All C. neoformans var. grubii and A. fumigatus isolates were considered wild-type for the azoles.

This experiment shows that Compound 1 (CMP1) was as active as anidulafungin (ANID), caspofungin (CAS), and micafungin (MCF) against common fungal organisms recovered from invasive fungal infections. The extended half-life profile is very desirable for prevention and treatment of serious fungal infections, especially in patients that can then be discharged.

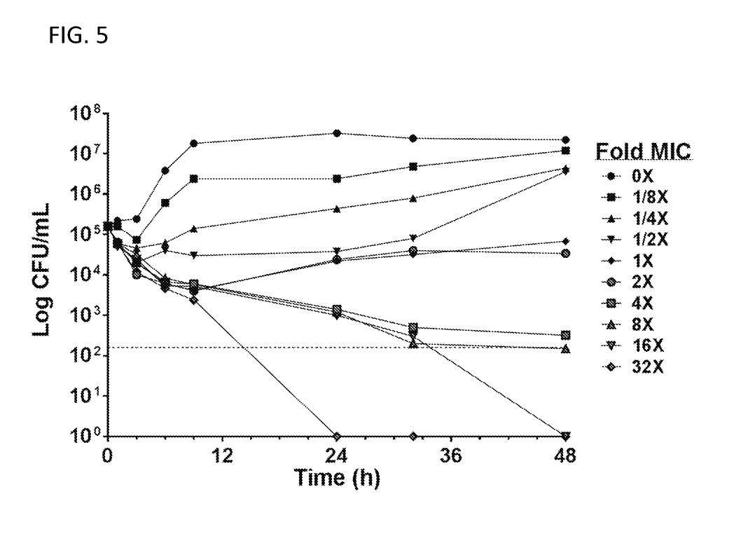

Furthermore, CMP1 displayed an MIC of 0.25 μg/mL against C. albicans ATCC 44858 (experiment was performed at pH 4). FIG. 5 shows the fungicidal properties of CMP1 against ATCC 44858.

### Example 7

### Compound 1 is Highly Efficacious Against Azole-Resistant Candida albicans in a Rat Model of Vulvovaginal Candidiasis

The efficacy of vaginally administered Compound 1 was compared to marketed miconazole and nystatin creams and oral fluconazole in an immunosuppressed rat model of VVC.

Groups of oophorohysterectomized female Wistar rats were used. Estradiol (ED) was administered at 10 mg/kg subcutaneously 3 days before C. albicans (R357) challenge then maintained with 4 mg/kg weekly injections throughout the study. Animals were immunosuppressed with dexamethasone applied in drinking water (2 mg/L) three days before challenge and throughout the study. To establish vaginal infection, anesthetized rats were inoculated intravaginally with C. albicans (10⁷ CFU) in PBS. All treatments began 48 hour after challenge. Compound 1 3% gel or 2% miconazole cream or nystatin cream were administered intravaginally at 0.1 mL/rat once daily for 3 days. For the gel, hydroxypropyl methylcellulose (0.4 g) was dissolved in 9.5 g of 50 mM sodium lactate buffer, pH 4.5. To this solution was added Compound 1 (0.3 g, acetate salt), methyl paraben (0.015 g), and EDTA disodium dihydrate (0.01 g). The solution was stirred until dissolution was complete. Sodium lactate buffer (50 mM, pH 4.2) was then added to bring the final volume to 10 mL.

Oral fluconazole was also administered at 20 mg/kg once daily for three days. Rats were sacrificed at two different time points after treatment end (days 5 and 12 corresponding to 1 and 8 days after treatment end) followed by vaginal lavage. C. albicans counts were measured in lavage fluid and also in excised vaginal tissue.

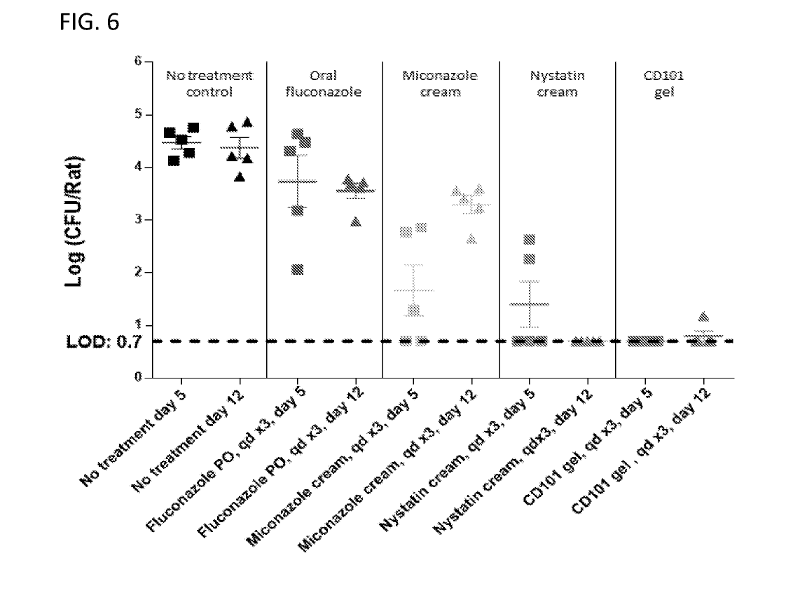

Results at days 5 and 12 (corresponding to 1 and 8 days after treatment end, respectively) are shown in FIG. 6. Compared with no treatment control, oral fluconazole showed minimal (<1 log-fold) reduction in vaginal CFU against the azole-resistant strain of C. albicans as expected. Topical administration of miconazole cream showed a >2 log-fold reduction in vaginal CFU one day after treatment end that was short-lived, as vaginal CFU rebounded a week later. Nystatin cream showed >2 log-fold reduction in vaginal CFU one day after treatment end that persisted through a week later. Compound 1 showed the greatest efficacy of the agents tested as vaginal CFUs were below the limit of detection (3.8 log-fold reduction) from one day after treatment end and remained >3 log-fold lower for a week after treatment end.

### Example 8

### Time-Kill Kinetics of Compound 1 for Azole-Susceptible and -Resistant Candida spp. at pH 4 in Vagina-Simulative Medium

The echinocandin class of antifungal agents has not been considered for treatment of VVC because currently available echinocandins are limited to IV administration. This study investigated the killing kinetics of the novel echinocandin, Compound 1, which is in development as a topical formulation, against Candida spp., including azole-S and -R strains, in conditions and at concentrations relevant to topical treatment of VVC.

Three strains (one azole-susceptible (azole-S) and two azole-resistant (azole-R)) were selected for 5 Candida spp. including C. albicans, C. glabrata, C. tropicalis, C. parapsilosis and C. krusei (which is intrinsically azole-R and so only two isolates were evaluated). Time-kill assays used a starting inoculum of mid-10⁵ colony-forming units (CFU)/mL in 10 mL of vagina-simulative medium (VSM) at pH 4.0. Compound 1 and an azole comparator, terconazole (TCZ), were used at fixed concentrations of 0, 2, 8, 32, and 128 μg/mL. Samples were taken at 0, 1, 3, 6, 9, 24, 48 and 72 h and were pelleted/washed 2× in PBS to remove residual drug then serial diluted in PBS and plated on SDA to enumerate CFU. Log-fold changes in CFU were calculated based on no-drug control groups. Cidality was defined as a ≥3-log reduction in CFU. Fungicidal activity was defined as a ≥3-log reduction in CFU from the starting assay inoculum.

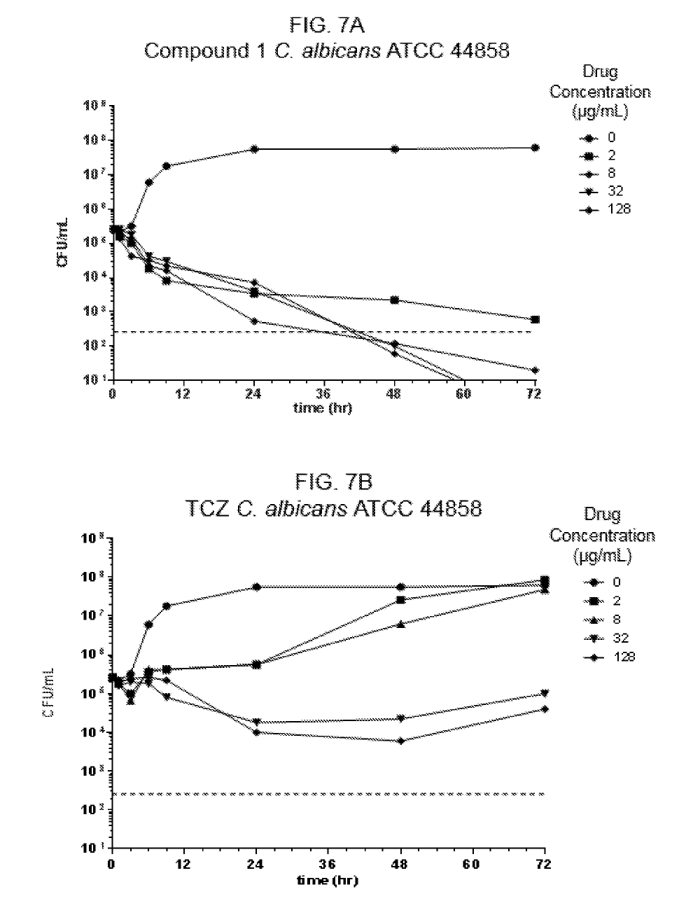

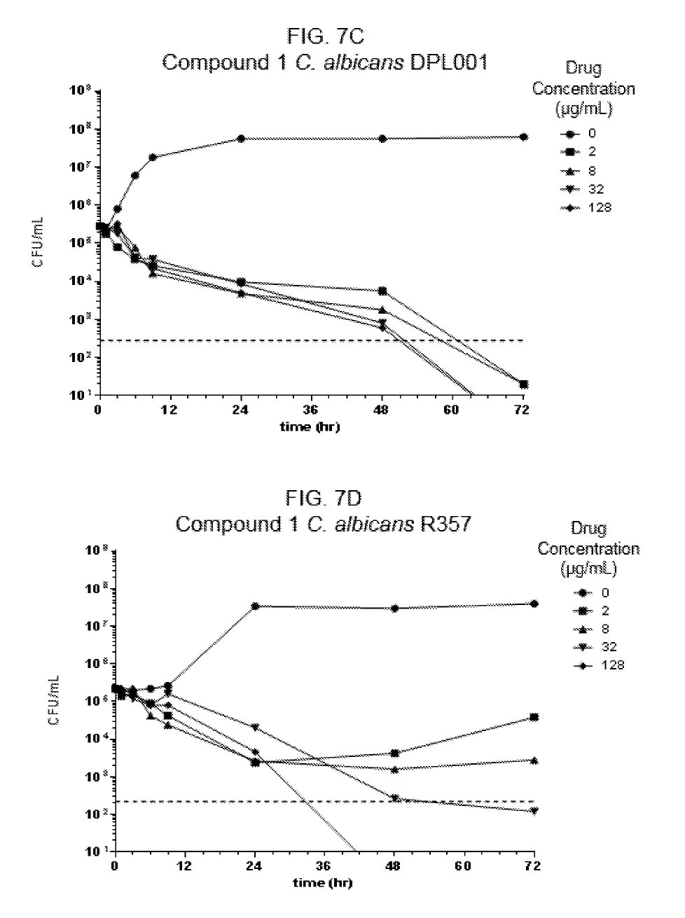

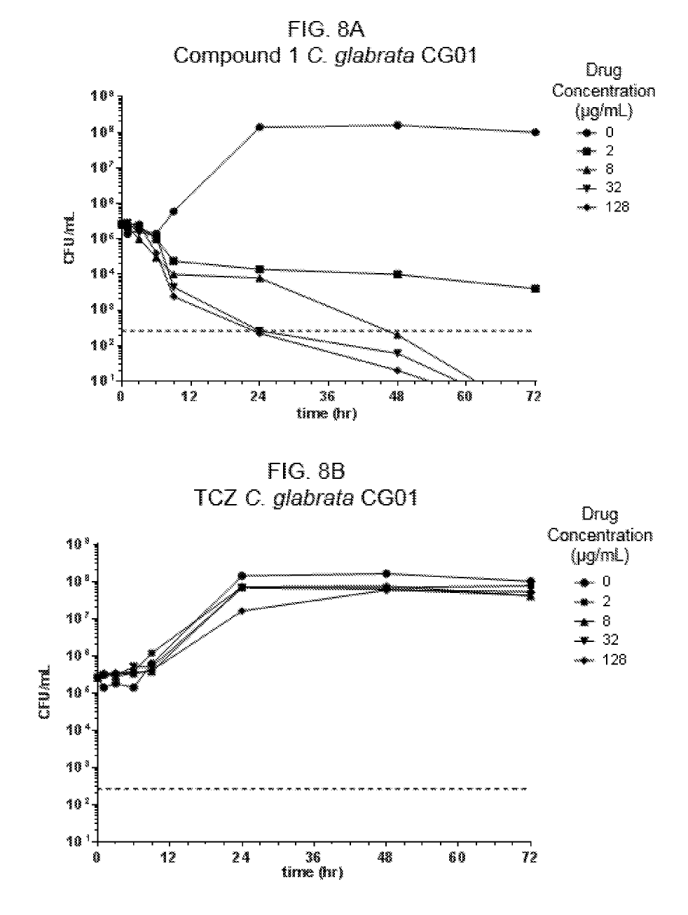

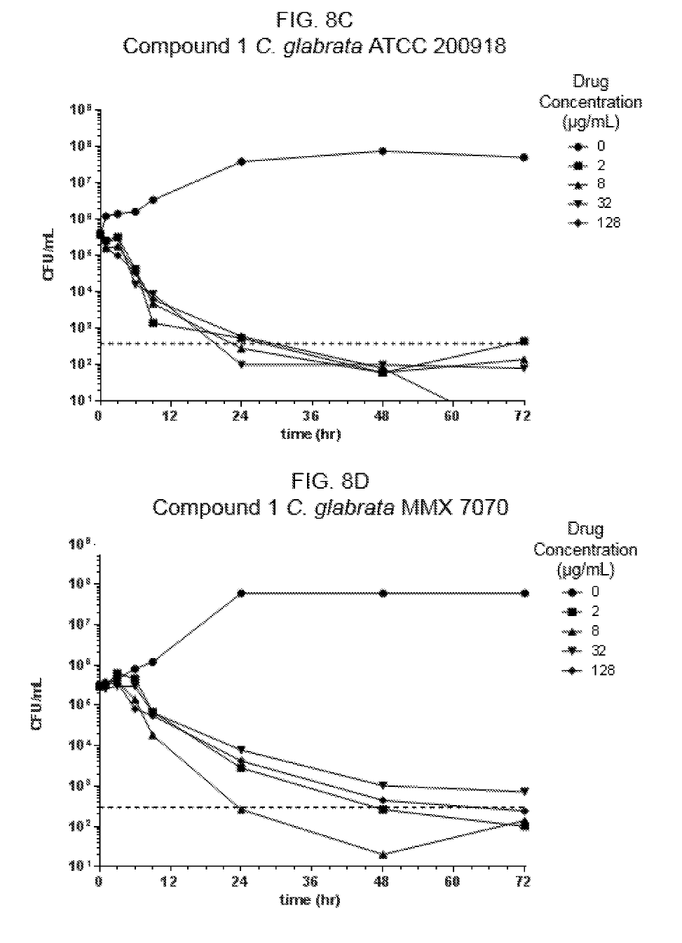

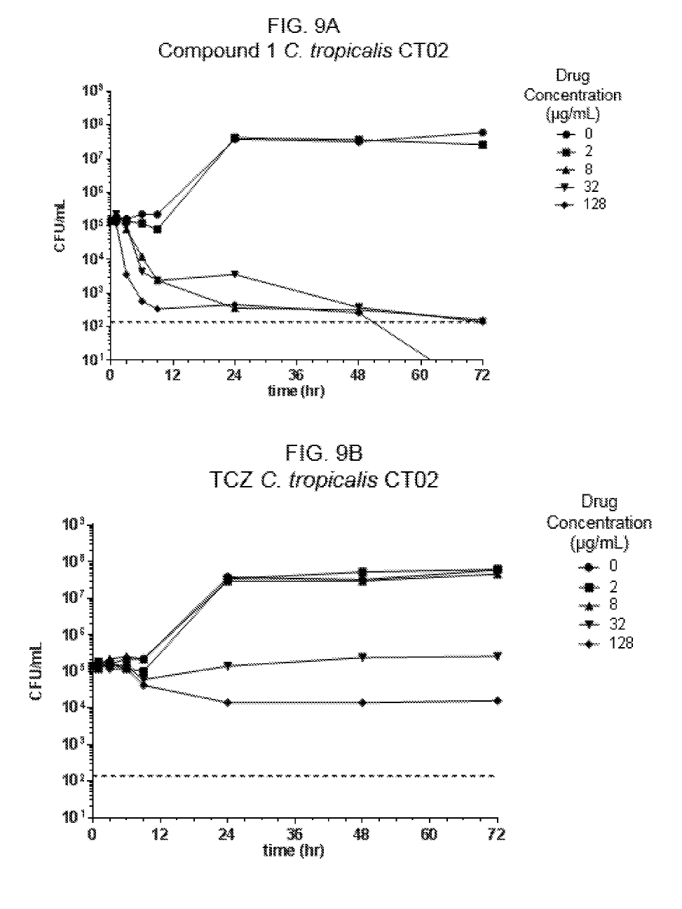

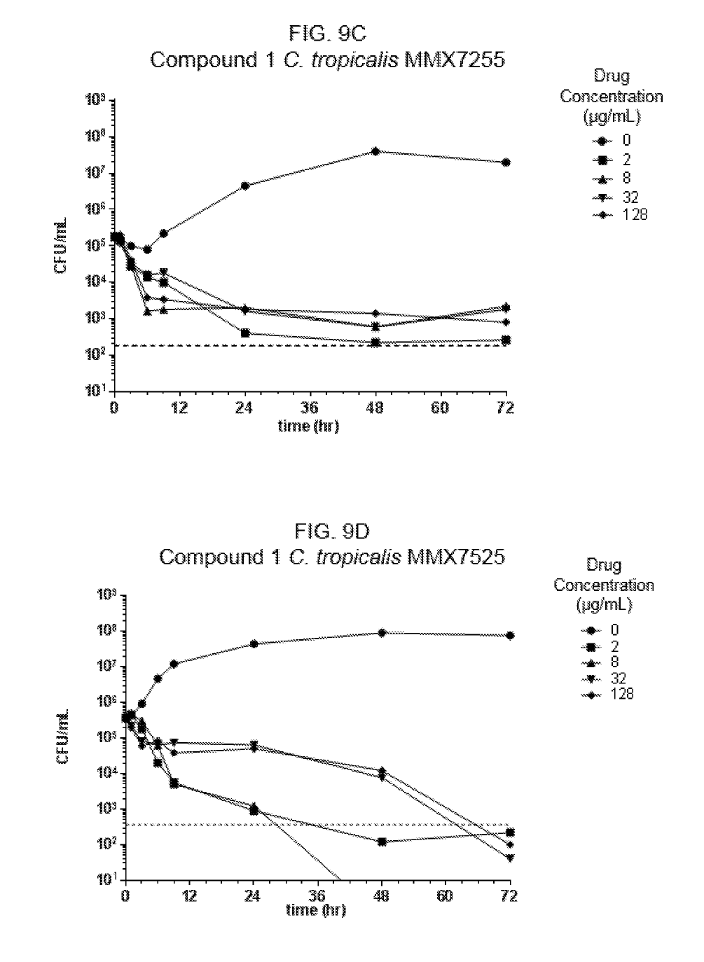

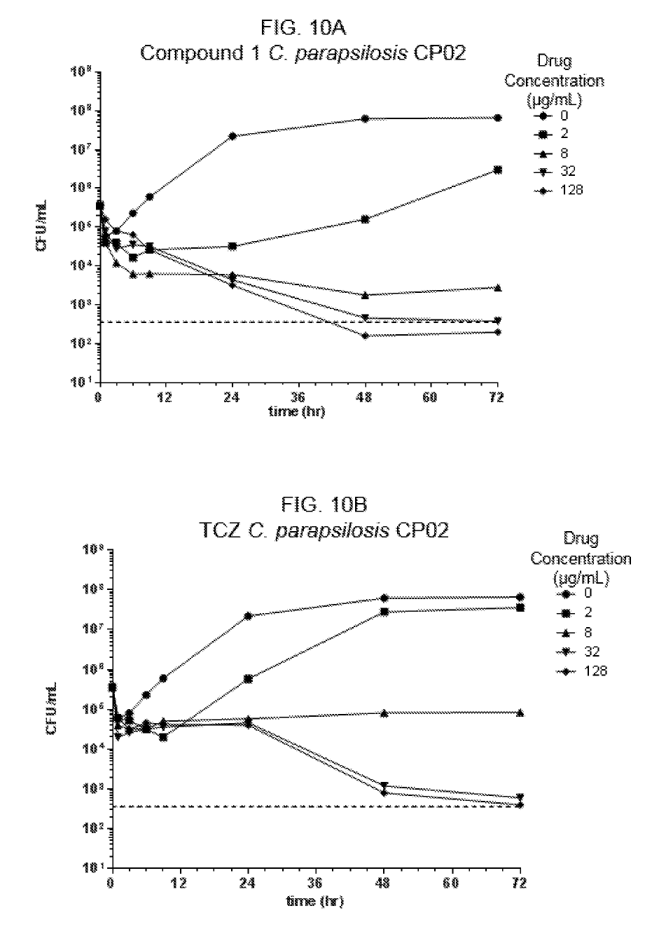

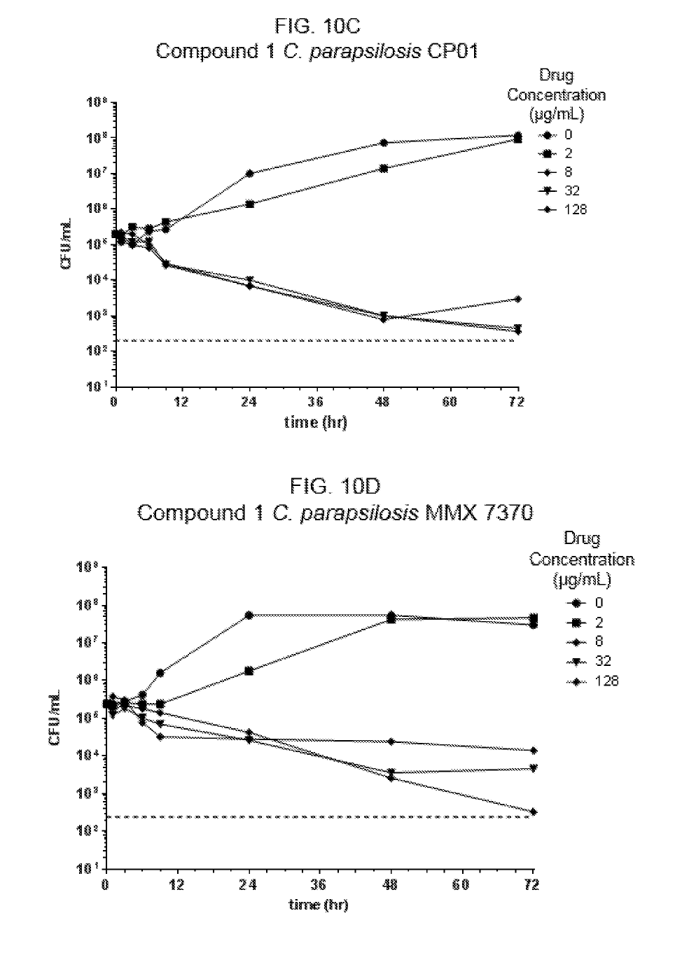

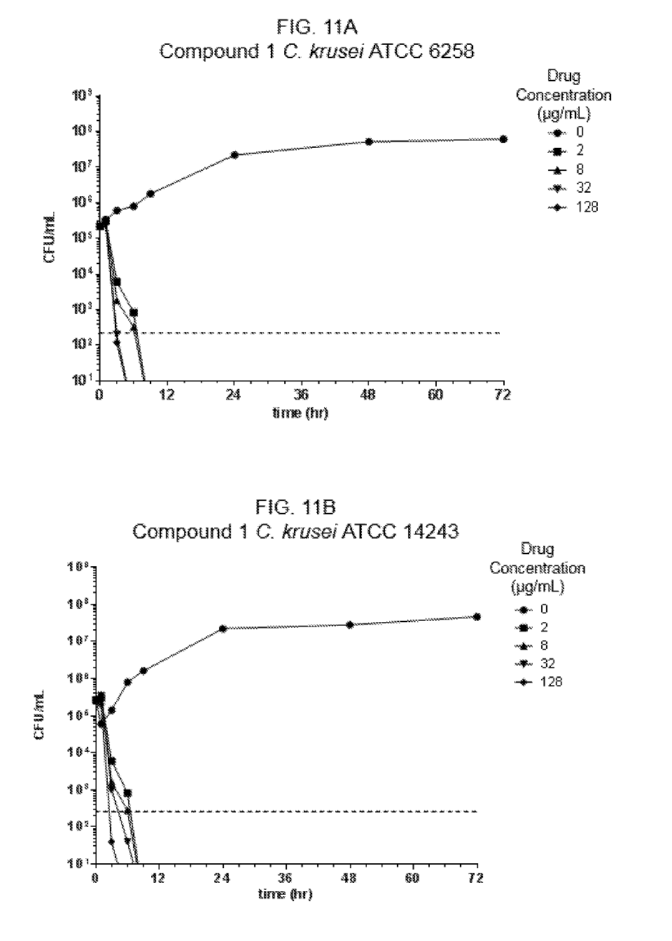

Compound 1 achieved cidality or near-cidality by 72 h for all strains in a strongly dose-dependent fashion, albeit to a lesser extent for C. tropicalis. The most rapid killing for Compound 1 occurred for the two C. krusei strains where cidality was achieved at all concentrations <24 h. Dose-dependent activity was also observed for TCZ. Even though TCZ was only tested against FLU-S strains, cidality was not achieved for any strain at any time point and typically only the highest concentrations resulted in CFU reductions. FIGS. 7A-7D, 8A-8D, 9A-9D, 10A-10D, and 11A-11B show the fungicidal properties of Compound 1 and TCZ against azole-S and azole-R strains of 5 Candida spp. (C. albicans, C. glabrata, C. tropicalis, C. parapsilosis and C. krusei). Tables 16-20 summarize the log-fold changes in CFU for each of the 5 Candida spp. (C. albicans, C. glabrata, C. tropicalis, C. parapsilosis, and C. krusei).

TABLE 16
C. albicans log-fold changes in CFU at 24, 48 and 72 hours

| | Susceptibility | | Time | Drug concentration (μg/mL) | | | | |
|---|---|---|---|---|---|---|---|---|
| Strain | to FLU (S/R) | Drug | (h) | 0 | 2 | 8 | 32 | 128 |
| C. albicans | S | Compound 1 | 24 | 2.33 | −1.88 | −2.68 | −1.81 | −1.56 |
| ATCC 44858 | | | 48 | 2.33 | −2.07 | −3.34 | −3.41 | −3.64 |
| | | | 72 | 2.38 | −2.64 | −4.11 | −5.41 | −5.41 |
| | | TCZ | 24 | 2.33 | 0.33 | 0.33 | −1.16 | −1.41 |
| | | | 48 | 2.33 | 2.00 | 1.38 | −1.07 | −1.64 |
| | | | 72 | 2.38 | 2.52 | 2.27 | −0.41 | −0.81 |
| C. albicans | R | Compound 1 | 24 | 2.30 | −1.46 | −1.77 | −1.51 | −1.75 |
| DPL001 | | | 48 | 2.30 | −1.70 | −2.19 | −2.54 | −2.67 |
| | | | 72 | 2.35 | −4.15 | −4.15 | −5.45 | −5.45 |
| C. albicans | R | Compound 1 | 24 | 2.19 | −1.96 | −1.93 | −1.04 | −1.68 |
| R357 | | | 48 | 2.13 | −1.72 | −2.14 | −2.93 | −5.34 |
| | | | 72 | 2.26 | −0.76 | −1.90 | −3.26 | −5.34 |

TABLE 17
C. glabrata log-fold changes in CFU at 24, 48 and 72 hours

| | Susceptibility | | Time | Drug concentration (μg/mL) | | | | |
|---|---|---|---|---|---|---|---|---|
| Strain | to FLU (S/R) | Drug | (h) | 0 | 2 | 8 | 32 | 128 |
| C. glabrata | S | Compound 1 | 24 | 2.73 | −1.27 | −1.51 | −3.00 | −3.07 |
| CG01 | | | 48 | 2.79 | −1.41 | −3.11 | −3.64 | −4.11 |
| | | | 72 | 2.59 | −1.81 | −5.41 | −5.41 | −5.41 |
| | | TCZ | 24 | 2.73 | 2.43 | 2.43 | 2.40 | 1.79 |
| | | | 48 | 2.79 | 2.42 | 2.45 | 2.36 | 2.35 |
| | | | 72 | 2.59 | 2.47 | 2.19 | 2.21 | 2.30 |
| C. glabrata | R | Compound 1 | 24 | 2.00 | −2.85 | −3.13 | −3.58 | −2.80 |
| ATCC | | | 48 | 2.29 | −3.80 | −3.80 | −3.58 | −3.68 |
| 200918 | | | 72 | 2.12 | −2.94 | −3.43 | −3.68 | −5.58 |
| C. glabrata | R | Compound 1 | 24 | 2.30 | −2.03 | −3.06 | −1.59 | −1.85 |
| MMX 7070 | | | 48 | 2.30 | −3.06 | −4.18 | −2.47 | −2.83 |
| | | | 72 | 2.30 | −3.48 | −3.33 | −2.62 | −3.10 |

TABLE 18
C. tropicalis log-fold changes in CFU at 24, 48 and 72 hours

| | Susceptibility to | | Time | Drug concentration (μg/mL) | | | | |
|---|---|---|---|---|---|---|---|---|
| Strain | FLU (S/R) | Drug | (h) | 0 | 2 | 8 | 32 | 128 |
| C. tropicalis | S | Compound 1 | 24 | 2.43 | 2.48 | −2.59 | −1.59 | −2.48 |
| CT02 | | | 48 | 2.36 | 2.41 | −2.64 | −2.57 | −2.73 |
| | | | 72 | 2.63 | 2.27 | −2.94 | −3.00 | −5.15 |
| | | TCZ | 24 | 2.43 | 2.39 | 2.33 | 0.00 | −1.00 |
| | | | 48 | 2.36 | 2.57 | 2.33 | 0.23 | −1.00 |
| | | | 72 | 2.63 | 2.65 | 2.52 | 0.27 | −0.94 |
| C. tropicalis | R | Compound 1 | 24 | 1.40 | −2.65 | −1.95 | −2.05 | −2.00 |
| MMX 7255 | | | 48 | 2.35 | −2.91 | −2.48 | −2.48 | −2.11 |
| | | | 72 | 2.05 | −2.84 | −1.91 | −2.00 | −2.35 |
| C. tropicalis | R | Compound 1 | 24 | 2.09 | −2.60 | −2.47 | −0.75 | −0.86 |
| MMX 7525 | | | 48 | 2.40 | −3.48 | −5.56 | −1.68 | −1.48 |
| | | | 72 | 2.32 | −3.21 | −5.56 | −3.95 | −3.56 |

TABLE 19
C. parapsilosis log-fold changes in CFU at 24, 48 and 72 hours

| | Susceptibility to | | Time | Drug concentration (μg/mL) | | | | |
|---|---|---|---|---|---|---|---|---|
| Strain | FLU (S/R) | Drug | (h) | 0 | 2 | 8 | 32 | 128 |
| C. parapsilosis | S | Compound 1 | 24 | 1.79 | −1.05 | −1.78 | −1.91 | −2.05 |
| CP02 | | | 48 | 2.24 | −0.35 | −2.30 | −2.89 | −3.35 |
| | | | 72 | 2.26 | 0.92 | −2.11 | −2.98 | −3.26 |
| | | TCZ | 24 | 1.79 | 0.21 | −0.79 | −0.89 | −0.95 |
| | | | 48 | 2.24 | 1.89 | −0.64 | −2.48 | −2.65 |
| | | | 72 | 2.26 | 2.00 | −0.63 | −2.78 | −2.95 |
| C. parapsilosis | R | Compound 1 | 24 | 1.70 | 0.85 | −1.46 | −1.30 | −1.47 |
| CP01 | | | 48 | 2.57 | 1.85 | −2.41 | −2.30 | −2.30 |
| | | | 72 | 2.78 | 2.67 | −1.83 | −2.66 | −2.74 |
| C. parapsilosis | R | Compound 1 | 24 | 2.35 | 0.88 | −0.93 | −0.97 | −0.76 |
| MMX 7370 | | | 48 | 2.35 | 2.24 | −1.00 | −1.82 | −1.97 |
| | | | 72 | 2.10 | 2.28 | −1.23 | −1.72 | −2.86 |

TABLE 20
C. krusei log-fold changes in CFU at 24, 48 and 72 hours

| | Susceptibility to | | Time | Drug concentration (μg/mL) | | | | |
|---|---|---|---|---|---|---|---|---|
| Strain | FLU (S/R) | Drug | (h) | 0 | 2 | 8 | 32 | 128 |
| C. krusei | R | Compound 1 | 24 | 2.00 | −5.34 | −5.34 | −5.34 | −5.34 |
| ATCC 6258 | | | 46 | 2.37 | −5.34 | −5.34 | −5.34 | −5.34 |
| | | | 72 | 2.45 | −5.34 | −5.34 | −5.34 | −5.34 |
| C. krusei | R | Compound 1 | 24 | 1.93 | −5.41 | −5.41 | −5.41 | −5.41 |
| ATCC | | | 46 | 2.03 | −5.41 | −5.41 | −5.41 | −5.41 |
| 14243 | | | 72 | 2.25 | −5.41 | −5.41 | −5.41 | −5.41 |

Compound 1, a novel echinocandin in development for topical administration, demonstrates potent activity against all major Candida spp. etiological agents of VVC, including azole-R strains, in in vitro time-kill assays performed under conditions relevant to the treatment of VVC. The activity of Compound 1 was more potent than that of TCZ for all strains evaluated.

### Example 9

### Assessment of Infection Following Azole-Resistant Candida albicans R357 Infection

The azole-resistant Candida albicans R357 was obtained from a frozen working stock and thawed at room temperature. A 0.1 mL aliquot of the stock was transferred to a sabouraud agar (SA) plate and incubated at 35-37° C. overnight. The culture was re-suspended in 1 mL cold PBS (>2.0×10⁹ CFU/mL, OD₆₂₀ 3.0-3.2) and diluted with PBS to target inoculum sizes of 5×10⁶, 5×10⁵, 5×10⁴, and 5×10³ CFU/mL. The actual colony counts were determined by plating dilutions to SA plates followed by 20-24 hr incubation.

Groups of male ICR (Institute of Cancer Research) mice (n=3 per group) weighing 22±2 g were used. Immune suppression was induced by two intraperitoneal injections of cyclophosphamide at 150 mg/kg 4 days (Day −4) and at 100 mg/kg 1 day (Day −1) before C. albicans infection. On Day 0, animals were intravenously inoculated (0.2 mL/mouse) with the R357 suspension. The animals were euthanized by CO₂ asphyxiation at 2 and 72 hr post-inoculation. A summary of the experimental design is shown in Table 21.

TABLE 21
Experimental Design

| | Inoculum size | Time at sacrifice | | ICR Mice |
|---|---|---|---|---|
| Group | (CFU/animal) | Post-infection | | (male) |
| 1a | 1E6 | 2 | hr | 3 |
| 1b | 1E6 | 72 | hr | 3 |
| 2a | 1E5 | 2 | hr | 3 |
| 2b | 1E5 | 72 | hr | 3 |
| 3a | 1E4 | 2 | hr | 3 |
| 3b | 1E4 | 72 | hr | 3 |
| 4a | 1E3 | 2 | hr | 3 |
| 4b | 1E3 | 72 | hr | 3 |

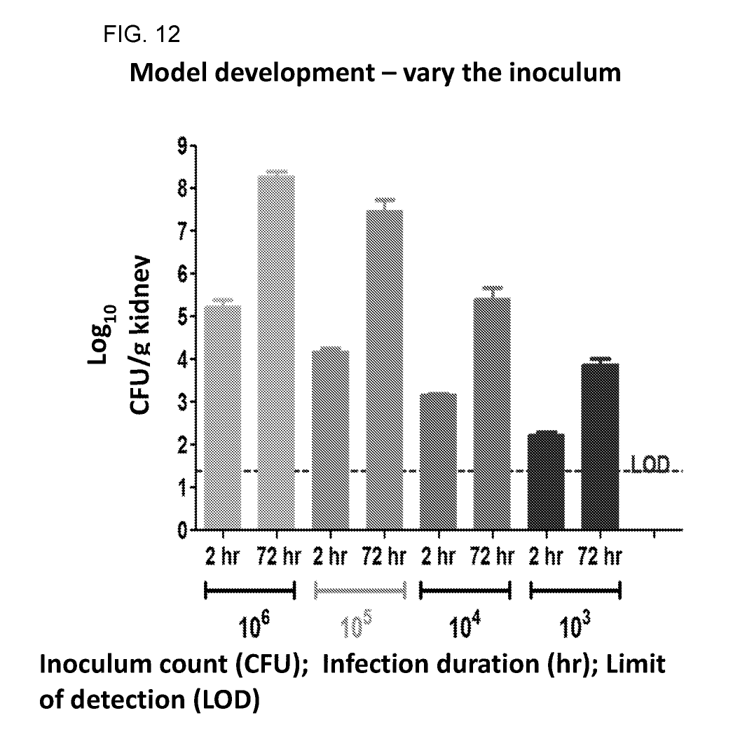

Paired kidneys were harvested and weighed. The harvested kidneys were homogenized in 1 mL sterile PBS (pH 7.4) and 10-fold dilutions were prepared and separately plated onto SA plates for further 20-24 hr incubation and then the fungal counts (CFU/g) in kidneys were calculated. Kidney fungal burdens from different inoculum densities of azole-resistant C. albicans strain R357 are shown in FIG. 12.

### Example 10

### Efficacy of Amphotericin B, Fluconazole, and Compound 1 in the Disseminated Infection Model With C. albicans R357

Materials

Test Articles. Compound 1 was dissolved in the vehicle containing 10% DMSO and 1% Tween 20 in 0.9% NaCl (see formulation table below). Amphotericin B and fluconazole were in powder form. Amphotericin B was dissolved in 0.9% NaCl. Fluconazole was dissolved in water (WFI: water for injection). A summary of the test articles is shown in Table 22.

TABLE 22
Test Articles

| | | | | Light | | Formulation |
|---|---|---|---|---|---|---|
| Test Article | Vehicle | Solubility^{a} | Color | Protection^{b} | Temp. | mg/mL |
| Compound 1 | 10% DMSO/1% Tween | S | colorless | Yes | 4° C. | 0.3, 1 and 3 |
| | 20 in 0.9% NaCl | | | | | |
| Amphotericin B | 0.9% NaCl | S | light yellow | Yes | 4° C. | 0.1 and 0.3 |
| Fluconazole | WFI | S | colorless | Yes | RT | 2 |

^{a}This is based on visual observation (S: soluble; SS: slightly soluble; I: insoluble (suspension or precipitation).
^{b}Test article is kept in tube or vial with brown color, or covered with aluminum foil.
c: 4° C.: prepared fresh and stored in the refrigerator or kept on ice; ET: prepared fresh and stored between 20-25° C.

Organism. The Candida albicans strains R357 was cryopreserved as single-use frozen working stock cultures stored at −70° C.

Animals. Male ICR mice weighing 22±2 g were acclimated for 3 days prior to use and were confirmed to be in good health. Space allocation for 3 or 5 animals was 27×20×14 cm. All animals were maintained in a hygienic environment with controlled temperature (20-24° C.), humidity (30%-70%) and 12 hours light/dark cycles. Free access to sterilized standard lab diet and autoclaved tap water were granted. All aspects of this work including housing, experimentation, and animal disposal were performed in general accordance with the “Guide for the Care and Use of Laboratory Animals: Eighth Edition” (National Academies Press, Wash., D.C., 2011).

Chemicals. Amphotericin B powder (Cat #A-9528, Sigma, USA), Bacto agar (Cat #214040, BD DIFCO, USA), cyclophosphamide (Cat #C-0768, Sigma, USA), dimethyl sulfoxide (Cat #1.02931.1000, Merck, Germany), fluconazole powder (Cat #F8929, SIGMA-Aldrich, USA), Fluid Sabouraud medium (Cat #264210, BD DIFCO, USA), Phosphate buffer saline (PBS) (Cat #P4417, Sigma, USA), Sodium chloride (Cat #S7653, SIGMA-Aldrich, USA), Tween 20 (Cat #P-7949, Sigma, USA) and Water for injection (WFI) (Tai-Yu, Taiwan).

Equipment. Biological safety cabinet (NuAire, USA), Absorbance microplate readers (Tecan, Infinite F50, USA), Centrifuge (Model 5922, Kubota, Japan), Individually Ventilated Cages (IVC, 36 Mini Isolator systems) (Tecniplast, Italy), Laminar flow (Tsao-Hsin, Taiwan), Orbital shaking incubator (Firstek Scientific, Taiwan), Pipetman (Rainin, USA), Polytron homogenizer (Kinematica, Switzerland) and Ultra-Low temperature freezer (NuAire, USA).

Methods

The azole-resistant Candida albicans (R357) strain was obtained from a frozen working stock and thawed at room temperature. A 0.1 mL aliquot stock was transferred to a sabouraud agar (SA) plate and incubated at 35-37° C. overnight. The culture was re-suspended in 1 mL cold PBS (>2.0×10⁹ CFU/mL, OD₆₂₀ 3.0-3.2) and diluted with PBS to 5×10⁵ CFU/mL. The actual colony counts were determined by plating dilutions to SA plates followed by 20-24 hr incubation. The actual inoculum count was 7.05×10⁵ CFU/mL.

Groups of male ICR mice (n=5 per group) weighing 22±2 g were used. Immune suppression was induced by two intraperitoneal injections of cyclophosphamide at 150 mg/kg 4 days (Day −4) and at 100 mg/kg 1 day (Day −1) before C. albicans infection. On Day 0, animals were intravenously inoculated (0.2 mL/mouse) with 5 inoculum sizes at 1.41×10⁵ CFU/0.2 mL/mouse of C. albicans (R357). Compound 1 (CMP1) was administered by intraperitoneal (IP) injection at 3, 10 and 30 mg/kg. Amphotericin B (AM-B) was administered by intravenous (IV) injection at 1 and 3 mg/kg. Fluconazole (FLU) was administered by oral gavage (PO) at 20 mg/kg. All test articles were administered once 2 hours after inoculation. The dosing volume was 10 mL/kg for all groups. A summary of the experimental design is shown in Table 23.

TABLE 23
Experimental Design

| | | Animal | Dose | Conc. | Dosage | | ICR Mice |
|---|---|---|---|---|---|---|---|
| Group | Test Article | Sacrifice | Route | mg/mL | mL/kg | mg/kg | (male) |
| 1 | N/A | 2 hr | — | — | — | — | 5 |
| 2 | Vehicle | 72 hr | IP | — | 10 | — | 5 |
| 3 | Vehicle | 48 hr | IP | — | 10 | — | 5 |
| 4 | Amphotericin B | 72 hr | IV | 0.1 | 10 | 1 | 5 |
| 5 | Amphotericin B | 48 hr | IV | 0.1 | 10 | 1 | 5 |
| 6 | Amphotericin B | 72 hr | IV | 0.3 | 10 | 3 | 5 |
| 7 | Amphotericin B | 48 hr | IV | 0.3 | 10 | 3 | 5 |
| 8 | Fluconazole | 72 hr | PO | 2 | 10 | 20 | 5 |
| 9 | Fluconazole | 48 hr | PO | 2 | 10 | 20 | 5 |
| 10 | Compound 1 | 72 hr | IP | 0.3 | 10 | 3 | 5 |
| 11 | Compound 1 | 48 hr | IP | 0.3 | 10 | 3 | 5 |
| 12 | Compound 1 | 72 hr | IP | 1 | 10 | 10 | 5 |
| 13 | Compound 1 | 48 hr | IP | 1 | 10 | 10 | 5 |
| 14 | Compound 1 | 72 hr | IP | 3 | 10 | 30 | 5 |
| 15 | Compound 1 | 48 hr | IP | 3 | 10 | 30 | 5 |

Target inoculum size 1E05 CFU/mouse (the actual inoculum size was 1.41E05 CFU/mouse).
Vehicle: 10% DMSO/1% Tween 20 in 0.9% NaCl
Test articles were dosed once 2 hrs after infection. Animals were sacrificed at assigned time points after infection.

The animals were euthanized by CO₂ asphyxiation 48 and 72 hr post-inoculation. Paired kidneys were harvested and weighed. The harvested kidneys were homogenized in 1 mL sterile PBS (pH 7.4) and 10-fold dilutions were prepared and separately plated onto SA plates. The fungal counts (CFU/g) in kidneys were calculated and the decrease percentage was calculated by the following formula:

Decrease (%)=[(CFU/g of vehicle−CFU/g of treatment)/(CFU/g of vehicle)]×100%

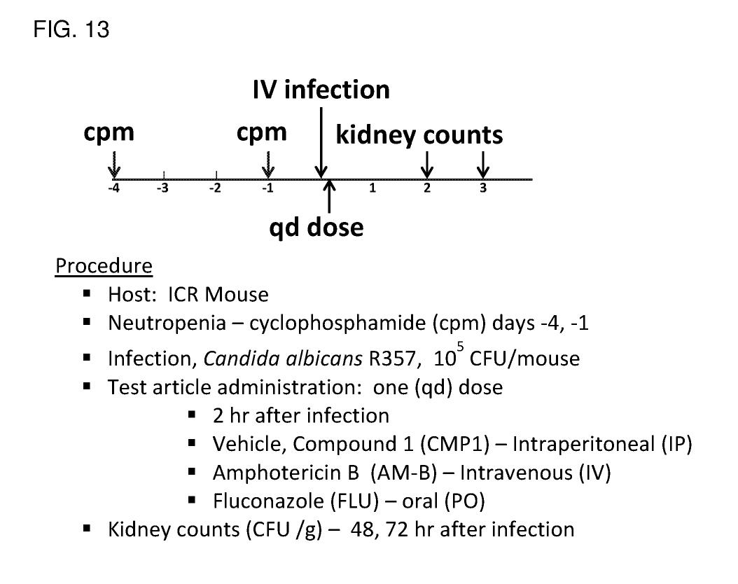

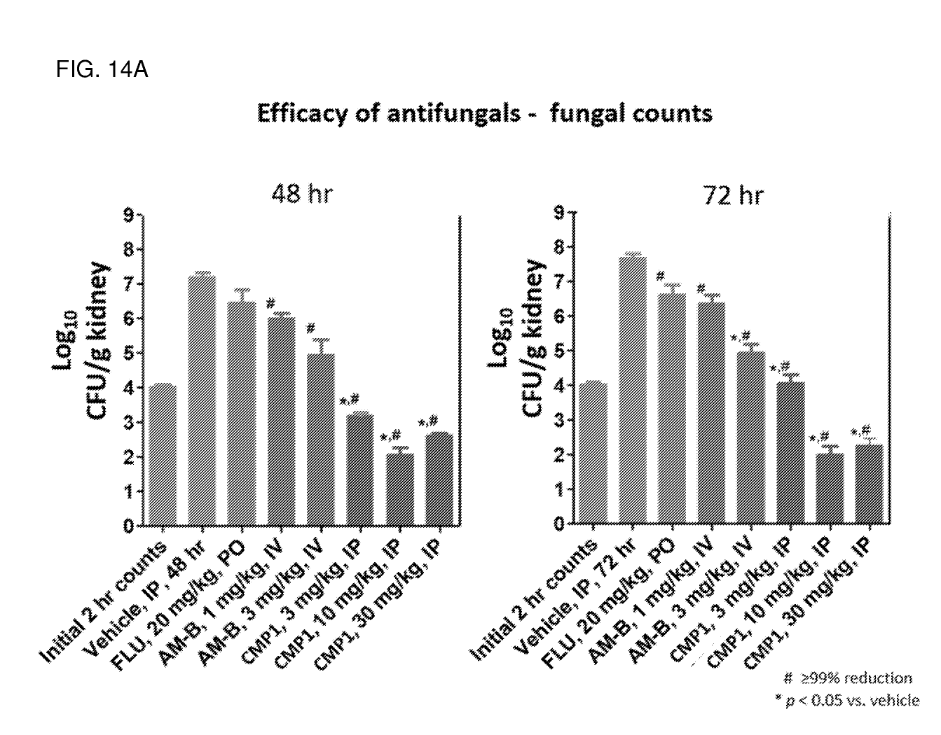

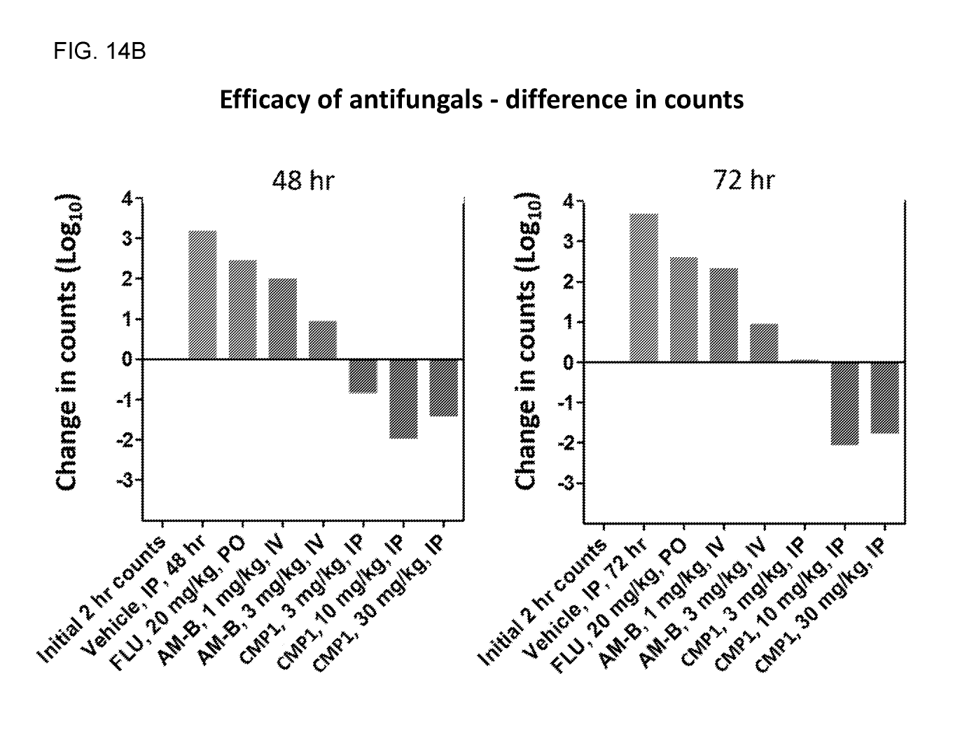

An outline of the experimental protocol is shown in FIG. 13. FIGS. 14A and 14B show the absolute fungal counts and the difference in fungal counts, respectively, of the test article treatment groups measured 48 or 74 hr after infection. A decrease of 99% or more (99%, 2-log) in the fungal counts of treated animals compared to those in the vehicle group measured 48 or 72 hr after infection indicated significant antimicrobial activity. One-way ANOVA followed by Dunnett's test was also applied to assess statistical significance.

Significant antimicrobial effects (P <0.05) were observed with Compound 1 treatment groups at 3, 10, and 30 mg/kg IP at 48 and 72 hr after infection. A two log reduction in fungal counts was observed with all Compound 1 treatment groups at the 48 and 72 hr time points. Significant effects were observed following amphotericin B treatment at 1 and 3 mg/kg IV at 48 and 72 hr after infection. Amphotericin B treatment at 3 mg/kg IV resulted in a two log reduction in counts at 72 hr time point. Administration of fluconazole at 20 mg/kg PO elicited a moderate reduction (51% and 84%) in colony counts 48 and 72 hr after infection compared to the vehicle control group that was not significant with one-way ANOVA followed by Dunnett's test analysis (P>0.05).

### Example 11

### Pharmacological Basis of Compound 1 Efficacy

Methods

Pharmacokinetic Study. Healthy female ICR mice were given a single dose of Compound 1 via intraperitoneal (IP) injection. The following doses, at three mice per dose, were studied: 1, 4, and 16 mg/kg. Compound 1 plasma concentrations were determined at 0, 1, 3, 6, 12, 24, 48, 72, 96 hours post-dose using a validated LC/MS assay with a lower limit of quantification of 0.02 μg/mL.

Dose-Fractionation Study. Male or female ICR mice (5 per regimen and observation time) weighing 22±2 g were rendered neutropenic for the study by injecting the mice with cyclophosphamide treatment four days (−Day 4) (150 mg/kg IP) and one day (−Day 1) prior to infection at 100 mg/kg IP. Neutropenia was sustained for the duration of the study with cyclophosphamide doses (100 mg/kg IP) every 48 hours on days +1, +3, +5 and +7 after infection. Each animal was inoculated intravenously with 1×10³ CFU of Candida albicans (Strain R303, MIC=0.125 mg/L). Compound 1 (or vehicle) was administered 24 hours post-infection via IP injection. The doses studied are shown in Table 24.

TABLE 24
Summary of Compound 1 dosing regimens evaluated

| Total Dose | Dosing Interval | Fractionated Doses |
|---|---|---|
| 0.7 mg/kg | Single Dose | 0.7 mg/kg × 1 |
| | Twice Weekly | 0.35 mg/kg × 2 |
| | Daily | 0.1 mg/kg × 7 |
| 2 mg/kg | Single Dose | 2 mg/kg × 1 |
| | Twice Weekly | 1 mg/kg × 2 |
| | Daily | 0.29 mg/kg × 7 |
| 7 mg/kg | Single Dose | 7 mg/kg × 1 |
| | Twice Weekly | 3.5 mg/kg × 2 |
| | Daily | 1 mg/kg × 7 |

Mice were sacrificed 168 hours (7 days) following the start of treatment. Control arm mice were sacrificed 0, 24, and 48 hours post administration of vehicle. Paired kidneys are aseptically harvested, homogenized, and plated for colony counts to determine the fungal burden (CFU/g).

Pharmacokinetic-Pharmacodynamic Analyses. Using the data collected from the PK study, a PK model was developed in S-ADAPT. Using the developed PK model, concentration-time profiles and AUC₀₋₁₆₈ₕ values were computed for each dosing regimen administered in the dose-fractionation study. Free-drug plasma concentrations were generated using a murine protein binding value of 99.1%. Relationships between the change in logio CFU from start of therapy and AUC₀₋₁₆₈ₕ were explored.

Results

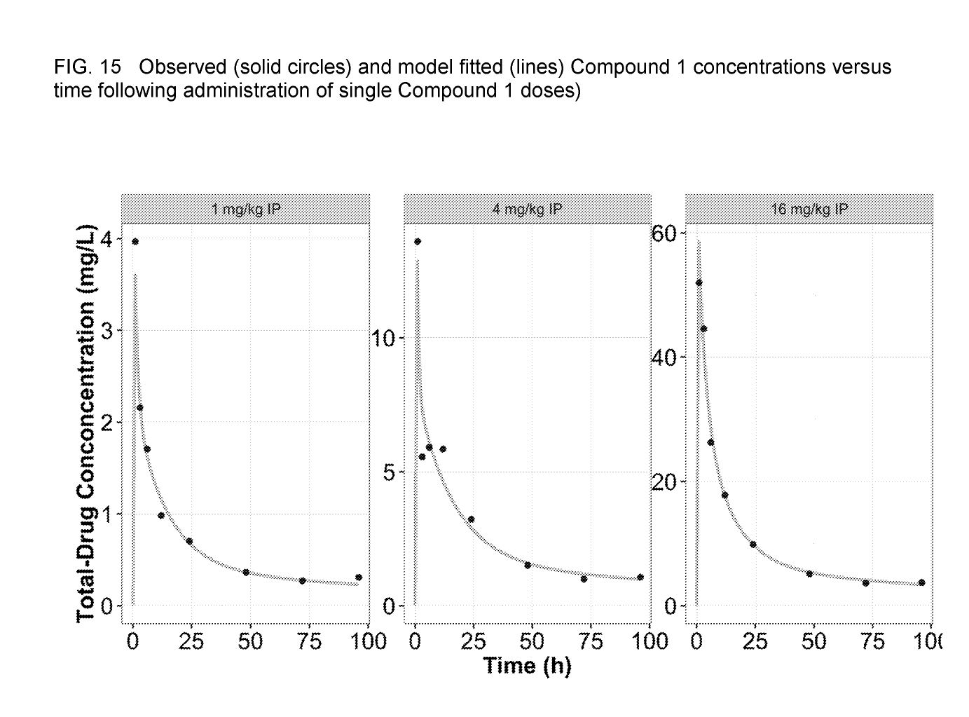

Compound 1 exhibited linear PK over the dose ranged studied (1 to 16 mg/kg IP). A 4-compartment model best described the PK data. Model fits are displayed in FIG. 15.

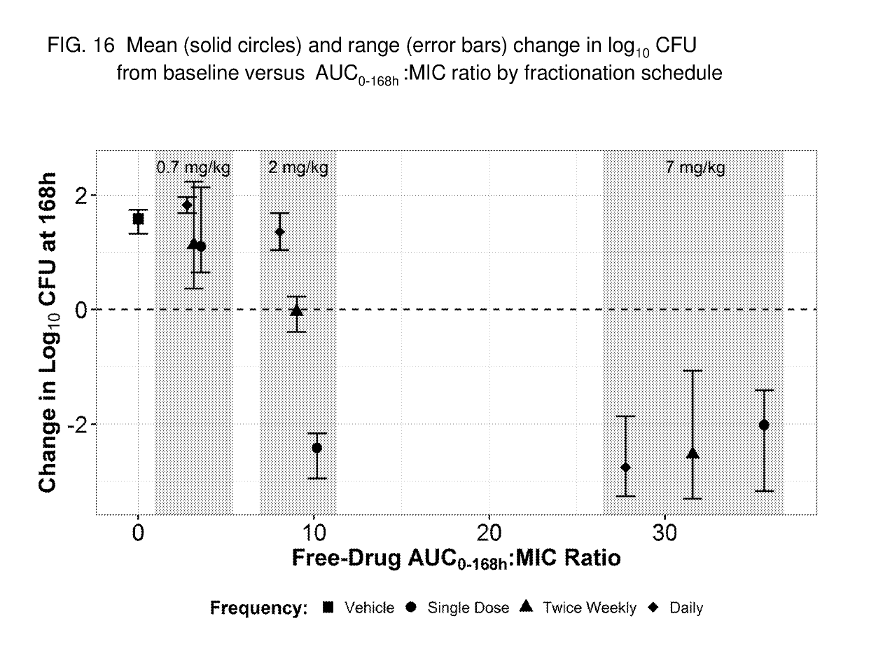

The results of the dose-fractionation study are displayed in FIG. 16, which shows that fungi grew well in the no-treatment control group. The magnitude of net change in fungal density (logio CFU) was similar regardless of fractionation schedule within the Compound 1 0.7 and 7 mg/kg dosing groups. However, results within the Compound 1 2 mg/kg group varied by the fractionation schedule.

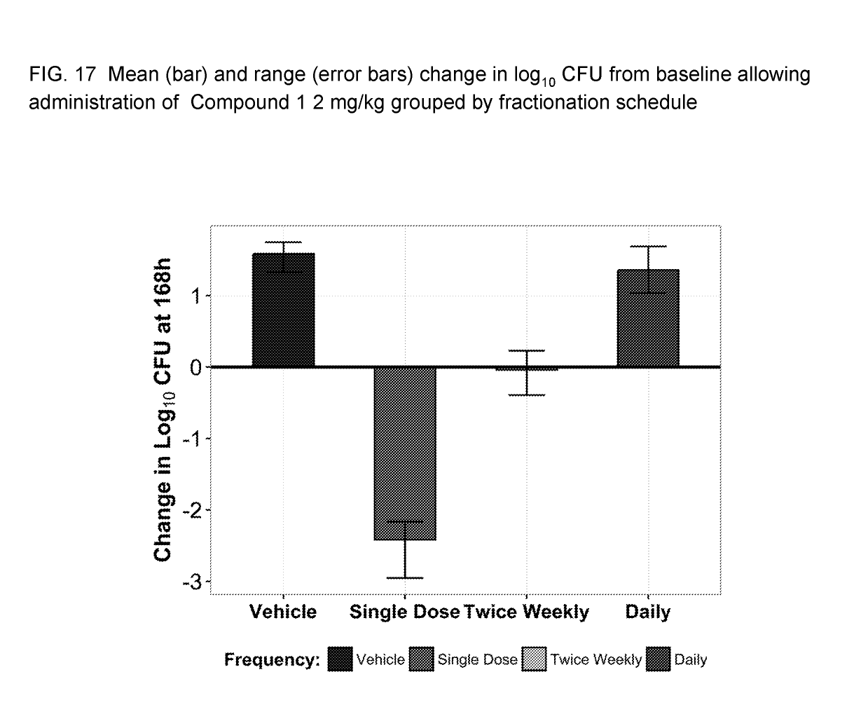

The change in logio CFU reduction from baseline at 168 hours by fractionation schedule for the Compound 1 2 mg/kg group is displayed in FIG. 17. When a total dose of 2 mg/kg was delivered daily (0.29 mg/kg/day), the magnitude of net change in fungal density (logio CFU) was similar to the no-treatment control group. However, when 2 mg/kg is delivered as a single dose, there was a greater than 2-logio CFU reduction from baseline at 168 hours. The 2 mg/kg×1 and 0.29 mg/kg daily×7 regimens had similar cumulative Compound 1 exposures at 168 hours, as displayed in FIG. 16. Despite having similar exposures, which influences efficacy, these regimens showed very different effects.

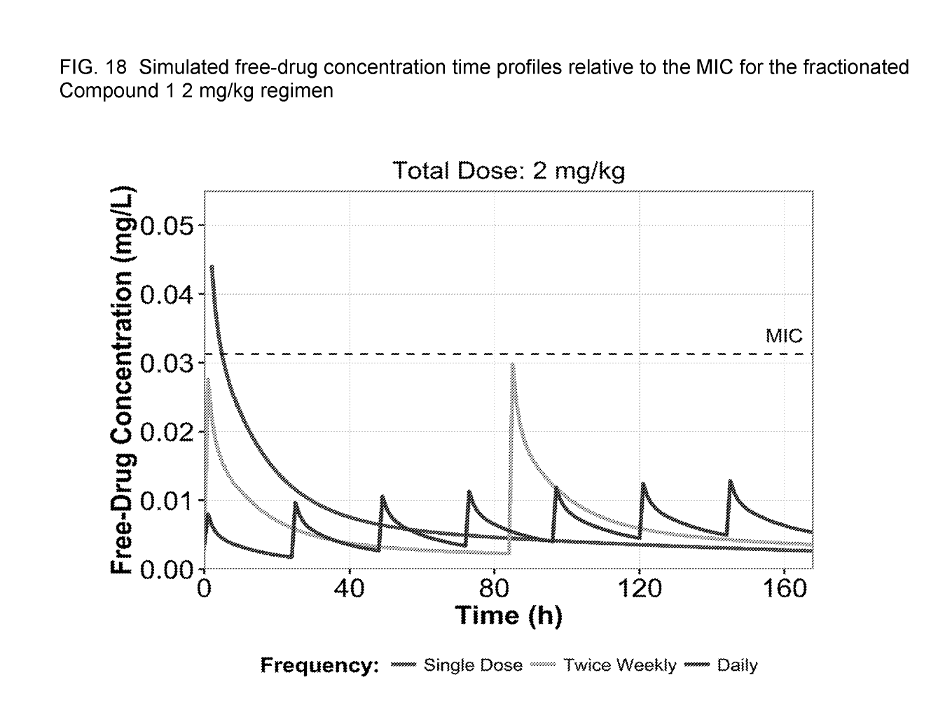

Free-drug plasma concentration-time profiles of the three fractionated Compound 1 2 mg/kg dosing regimens are displayed in FIG. 18. All three regimens display very different exposure profiles. In particular, the single dose regimen results in larger Compound 1 exposures early in therapy. Free-drug plasma AUC₀₋₂₄ is 0.0654, 0.0303, and 0.00948 mg·h/L following administration of Compound 1 2 mg/kg as a single dose, twice weekly, and daily regimen, respectively. Further, administration of a single dose results in free-drug plasma concentrations which remain above those for the twice weekly and daily regimens for 84 and 48 hours, respectively.

Three Compound 1 regimens with similar total exposures, yet very different exposure shapes, display considerably different efficacy. This suggests that the shape of the Compound 1 AUC is a determinant of efficacy, with front loaded regimens demonstrating greater efficacy. The magnitude of the net change in fungal burden was similar regardless of fractionation schedule within the Compound 1 0.7 and 7 mg/kg dosing groups, but differed within the 2 mg/kg group. A 2 mg/kg dose was considerably more effective when given once per week compared to the same dose divided into twice-weekly or daily regimens.

### Example 12

### Efficacy of Amphotericin B, Fluconazole, and Compound 1 in Mouse Models of Aspergillosis and Azole-Resistant Disseminated Candidiasis

Methods

An azole-resistant strain of Candida albicans (R357; resistant to fluconazole (FLU), voriconazole, and posaconazole, but susceptible to amphotericin B (AMB) and echinocandins) isolated from human blood was used for the mouse candidiasis model. A test strain of Aspergillus fumigatus (ATCC 13073) was used for the mouse aspergillosis model. Mice were rendered neutropenic by cyclophosphamide and then infected by injections of C. albicans (10⁵ CFU/mouse) or A. fumigatus (10⁴ CFU/mouse) into the tail vein. Test articles were administered starting 2 hours after infection. In the mouse candidiasis model, groups of 5 mice each received one dose of AMB (3 mg/kg IV), FLU (20 mg/kg orally), or Compound 1 (3, 10, or 30 mg/kg by intraperitoneal administration (IP)). After 72 hours post-infection, mice were euthanized and C. albicans counts in kidney tissue (CFU/g) were measured. In the mouse aspergillosis model, groups of 10 mice each received one dose of AMB (2 mg/kg IP) or Compound 1 (2 mg/kg IV and IP). Survival was monitored daily for 10 days. Differences between vehicle and test article groups were assessed for significance by one-way ANOVA followed by Dunnett's test and Fisher's Exact test in the candidiasis and aspergillosis models, respectively.

Results

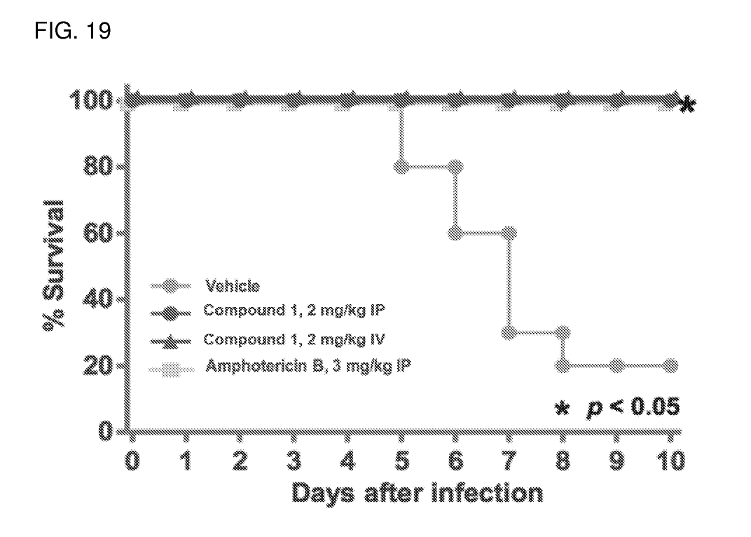

Compound 1 administered at 3 mg/kg produced a >99.9% (or >3-log; P<0.001) reduction in C. albicans CFUs compared with vehicle through at least 72 hours post-dose following a single IP dose. AMB showed similar, albeit less robust, efficacy (>99% or >2-log reduction in CFU; P<0.05), whereas fluconazole was less efficacious (83.9% or <2-log reduction in CFU). In the aspergillosis model, Compound 1 administered 2 mg/kg IV or IP showed similar efficacy to that of AMB 2 mg/kg IP, both with significantly longer survival than vehicle (P<0.05; see FIG. 19).

Conclusions

A single dose of Compound 1 administered at 3 mg/kg produced significant reduction in C. albicans burden compared with vehicle (P<0.001) in the neutropenic mouse model of azole-resistant candidiasis, demonstrating efficacy comparable, if not better, to that of AMB at the same dose. One dose of Compound 1 also demonstrated efficacy in the mouse model of aspergillosis. These data support the efficacy of Compound 1 for treatment of serious infections caused by Candida, including azole-resistant strains, and Aspergillus spp.

### Example 13

### Pharmacokinetics and Target Attainment of Single and Multiple Doses of Compound 1 by IV Administration

Compound 1 has shown robust efficacy in neutropenic mouse infection models of disseminated candidiasis, as well as aspergillosis, and an excellent nonclinical safety/toxicology profile. Two randomized, double-blind, placebo-controlled, phase 1, dose-escalation trials were conducted to establish the pharmacokinetics (PK) of single and multiple weekly dosing of Compound 1 after IV administration. PK-pharmacodynamic (PD) target attainment analyses of these data were conducted.

Methods

Sequential cohorts of 8 healthy human subjects (n=6, Compound 1; n=2, placebo) received a single dose of Compound 1 (50, 100, 200, 400 mg) or multiple weekly doses (100 mg×2, 200 mg×2, 400 mg×3) infused intravenously over 1 hour. PK was assessed using plasma and urine samples collected over 21 days. Safety and tolerability were assessed by adverse events (AEs), vital signs, physical exams, electrocardiograms (ECGs), and safety laboratory values up to 21 days after dosing. Data from these clinical trials were used to develop a population PK model and perform Monte Carlo simulation (n=2000) evaluating a single dose of 400 mg and multiple weekly doses (400 mg×3 or 400 mg×1 followed by 200 mg×2) chosen for an upcoming Phase 2, dose-ranging trial in candidemia. For each dosing regimen, percent probabilities of PK-PD target attainment against Candida albicans were calculated.

Results

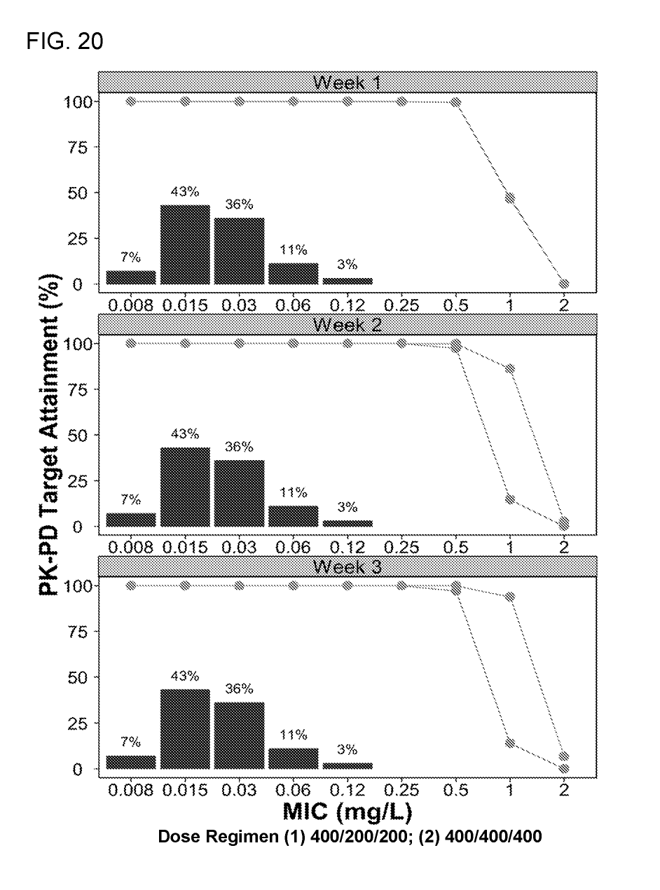

Compound 1 administered by IV infusion and placebo groups had similar incidences of AEs. The majority were mild, and all resolved completely. Slightly higher incidences of AEs and mild transient infusion reactions were seen in the group that received Compound 1 400 mg×3 weekly doses. There were no clinically significant safety issues in observed or laboratory assessments, and no deaths, serious AEs, severe AEs, or withdrawals due to an AE. Compound 1 administered IV demonstrated dose-proportional plasma exposures, low apparent clearance (<0.3 L/h), long half-life (t_{1/2}>80 h), minimal urinary excretion (<1%), and minor accumulation (30% to 55%, multiple-dose study). Target attainment (% probability) against Candida albicans was 100% for both weekly dosing regimens of Compound 1 (see FIG. 20) and >99% for the single dose of Compound 1 400 mg.

Conclusion

Compound 1 IV was safe and well tolerated as single and multiple doses up to 400 mg once weekly for up to 3 weeks. Target attainment analyses support the dosing regimens evaluated. The high plasma exposures achieved with Compound 1 IV may improve treatment outcomes compared to other echinocandins, and its long t_{1/2} enables weekly dosing. These findings help to establish PK-PD optimized drug exposures (e.g., Compound 1 IV dosed once-weekly), while reducing the resources required for therapeutic drug monitoring.

What is claimed is:

1. A method of treating a drug-resistant fungal infection in a subject comprising administering two or more doses of a salt of Compound 1, 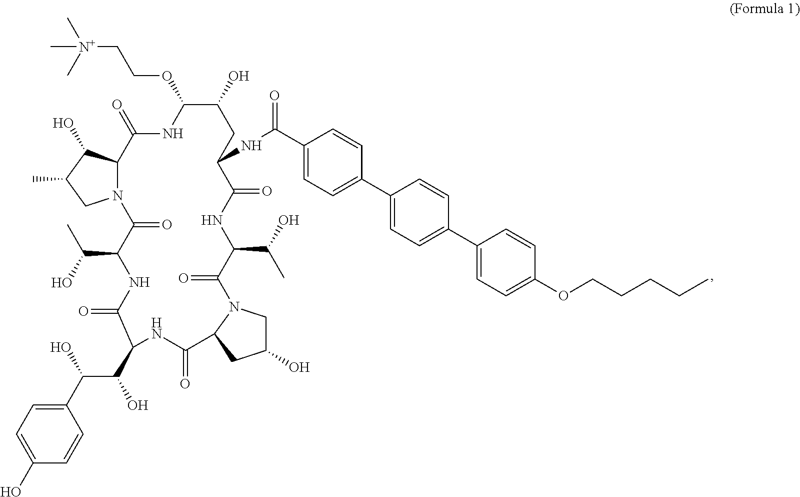 [SMILES: CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1]
or a neutral form thereof, to said subject in an amount and for a duration sufficient to treat said drug-resistant fungal infection.

2. A method of treating a fungal infection in a subject who has failed treatment with an antifungal therapy, said method comprising administering a salt of Compound 1, or a neutral form thereof, to said subject in an amount and for a duration sufficient to treat said fungal infection.

3. The method of claim 1 or 2, wherein said fungal infection is caused by a fungus having a mutant 1,3-β-D-glucan synthase enzyme complex.

4. A method of treating a fungal infection in a subject comprising administering a salt of Compound 1, or a neutral form thereof, to said subject, in an amount and for a duration sufficient to treat said fungal infection, wherein said fungal infection is caused by a fungus having a mutant 1,3-β-D-glucan synthase enzyme complex.

5. The method of any one of claims 1-4, wherein the administering step comprises intravenously administering doses of about 150 mg to about 800 mg of a salt of Compound 1, or a neutral form thereof, to said subject, wherein two or more doses are administered to said subject over a period of 1 to 4 weeks.

6. A method of treating a fungal infection in a subject comprising intravenously administering doses of about 550 mg to about 800 mg of a salt of Compound 1, or a neutral form thereof, to said subject, wherein two or more doses are administered to said subject over a period of 1 to 4 weeks.

7. A method of treating a fungal infection in a subject comprising intravenously administering doses of about 150 mg to about 800 mg of a salt of Compound 1, or a neutral form thereof, one to three times per week to said subject for 2 to 4 weeks.

8. A method of treating a fungal infection in a subject comprising intravenously administering two or more doses of a composition comprising about 150 mg to about 800 mg of a salt of Compound 1, or a neutral form thereof, to said subject.

9. The method of any of claims 6-8, wherein the administered amount maintains at least a mutant prevention concentration of Compound 1 in the plasma of the subject for a period of at least 8 hours.

10. The method of any one of claims 1-9, wherein said fungal infection is an echinocandin-resistant, polyene-resistant, flucytosine-resistant, or azole-resistant fungal infection.

11. The method of claim 10, wherein said fungal infection is an echinocandin-resistant infection.

12. The method of any one of claims 1-11, wherein said subject has failed treatment with an echinocandin therapy.

13. The method of claim 12, wherein said subject has failed treatment with anidulafungin, micafungin, or caspofungin.

14. The method of any one of claims 1-11, wherein said subject has failed treatment with a polyene therapy, flucytosine therapy, or an azole therapy.

15. The method of any one of claims 1-14, wherein said fungal infection is caused by a fungus having a mutant 1,3-β-D-glucan synthase enzyme complex comprising one or more mutations in FKS genes.

16. The method of any one of claims 1-15, wherein said fungal infection is a Candida infection.

17. The method of claim 16, wherein said Candida is selected from the group consisting of Candida albicans, C. glabrata, C. dubliniensis, C. krusei, C. parapsilosis, C. tropicalis, C. orthopsilosis, C. guilliermondii, C. rugosa, and C. lusitaniae.

18. The method of claim 17, wherein said Candida is Candida albicans.

19. The method of claim 17, wherein said Candida is Candida glabrata.

20. The method of any one of claims 16-19, wherein said Candida infection is candidemia, oropharyngeal candidiasis, esophageal candidiasis, mucosal candidiasis, genital candidiasis, vulvovaginal candidiasis, gastrointestinal candidiasis, rectal candidiasis, hepatic candidiasis, renal candidiasis, pulmonary candidiasis, splenic candidiasis, cardiovascular candidiasis, or invasive candidiasis.

21. The method of claim 20, wherein said cardiovascular candidiasis is endocarditis.

22. The method of any one of claims 1-15, wherein said fungal infection is an Aspergillus infection.

23. The method of any one of claims 1-15, wherein said fungal infection is a dermatophyte infection.

24. The method of any one of claim 1-4 or 9-23, wherein the administering step comprises administering a salt of Compound 1, or a neutral form thereof, topically, intravaginally, intraorally, intravenously, intramuscularly, intradermally, intraarterially, subcutaneously, orally, or by inhalation.

25. The method of claim 24, wherein the administering step comprises administering a salt of Compound 1, or a neutral form thereof, intravenously.

26. A method of killing an echinocandin-resistant, polyene-resistant, flucytosine-resistant, or azole-resistant Candida comprising exposing the echinocandin-resistant, polyene-resistant, flucytosine-resistant, or azole-resistant Candida to a salt of Compound 1, or a neutral form thereof, in an amount and for a duration sufficient to kill the echinocandin-resistant, polyene-resistant, flucytosine-resistant, or azole-resistant Candida.

27. The method of claim 26, wherein said Candida is selected from the group consisting of Candida albicans, C. glabrata, C. dubliniensis, C. krusei, C. parapsilosis, C. tropicalis, C. orthopsilosis, C. guilliermondii, C. rugosa, and C. lusitaniae.

28. The method of claim 27, wherein said Candida is Candida albicans.

29. The method of claim 27, wherein said Candida is Candida glabrata.

30. A method of treating an echinocandin-resistant fungal infection in a subject comprising intravenously administering two or more doses of a salt of Compound 1, or a neutral form thereof, to said subject in an amount sufficient to treat said echinocandin-resistant fungal infection over a period of 1 to 4 weeks, wherein the echinocandin-resistant fungal infection is caused by a fungus having a mutant 1,3-β-D-glucan synthase enzyme complex comprising one or more mutations in FKS genes.

31. A method of treating a fungal infection in a subject comprising intravenously administering two or more doses of about 150 mg to about 800 mg of Compound 1 in salt or neutral form, to said subject in a dosing regimen that maintains at least a mutant prevention concentration of Compound 1 in the plasma of the subject for a period of at least 8 hours.

32. The method of any one of claim 1-5 or 7-31, wherein the method comprises weekly dosing of Compound 1 in salt or neutral form, the method comprising
(a) intravenously administering a first dose of about 400 mg of Compound 1 in salt or neutral form,
(b) intravenously administering a subsequent dose of about 200 mg of Compound 1 in salt or neutral form, and
(c) optionally repeating step (b) for one to ten weeks.

33. The method of claim 32, wherein the method consists of
(a) intravenously administering a first dose of about 400 mg of Compound 1 in salt or neutral form,
(b) intravenously administering a second dose of 200 mg of Compound 1 in salt or neutral form, and
(c) optionally intravenously administering a third dose of about 200 mg of Compound 1 in salt or neutral form,
wherein the first dose is administered on day 1, the second dose is administered on day 8, and the third dose, if administered, is administered on day 15.

34. The method of any one of claim 1-5 or 7-31, wherein the method comprises weekly dosing of Compound 1 in salt or neutral form, the method comprising
(a) intravenously administering a dose of about 400 mg of Compound 1 in salt or neutral form, and
(b) intravenously administering a subsequent dose of about 400 mg of Compound 1 in salt or neutral form, and
(c) optionally repeating step (b) for one to ten weeks.

35. The method of claim 34, wherein the method consists of
(a) intravenously administering a first dose of about 400 mg of Compound 1 in salt or neutral form,
(b) intravenously administering a second dose of about 400 mg of Compound 1 in salt or neutral form, and
(c) optionally intravenously administering a third dose of about 400 mg of Compound 1 in salt or neutral form,
wherein the first dose is administered on day 1, the second dose is administered on day 8, and the third dose, if administered, is administered on day 15.

36. The method of claim 33, wherein the third dose of about 200 mg of Compound 1 in salt or neutral form is administered if on day 15 mycological eradication and/or clinical cure is not achieved in the subject.

37. The method of claim 35, wherein the third dose of about 400 mg of Compound 1 in salt or neutral form is administered if on day 15 mycological eradication and/or clinical cure is not achieved in the subject.

38. The method of claim 36 or 37, wherein mycological eradication is determined by two negative blood cultures drawn at 12 hours apart without intervening positive blood cultures.

39. The method of claim 33, wherein the third dose of about 200 mg of Compound 1 in salt or neutral form is administered if on day 15 the subject displays symptoms of a fungal infection.

40. The method of claim 35, wherein the third dose of about 400 mg of Compound 1 in salt or neutral form is administered if on day 15 the subject displays symptoms of a fungal infection.

41. The method of claim 39 or 40, wherein symptoms of the fungal infection comprises fever, cough, shortness of breath, weight loss, and/or night sweats.

42. A method of administering Compound 1 to a subject, the method consisting of
(a) intravenously administering a first dose comprising 400 mg of Compound 1 in salt or neutral form,
(b) intravenously administering a second dose comprising 200 mg of Compound 1 in salt or neutral form, and
(c) optionally intravenously administering a third dose comprising 200 mg of Compound 1 in salt or neutral form,
wherein the first dose is administered on day 1, the second dose is administered on day 8, and the third dose, if administered, is administered on day 15.

43. A method of administering Compound 1 to a subject, the method consisting of
(a) intravenously administering a first dose comprising 400 mg of Compound 1 in salt or neutral form,
(b) intravenously administering a second dose comprising 400 mg of Compound 1 in salt or neutral form, and
(c) optionally intravenously administering a third dose comprising 400 mg of Compound 1 in salt or neutral form,
wherein the first dose is administered on day 1, the second dose is administered on day 8, and the third dose, if administered, is administered on day 15.

44. The method of any one of claims 1-43, wherein Compound 1 is administered over a time period of 30 to 180 minutes (e.g., 60±5 minutes).

45. The method of any one of claims 1-44, wherein the Compound 1 is administered as an aqueous pharmaceutical composition.

46. The method of claim 45, wherein the pharmaceutical composition has a pH of from 4.0 to 8.

47. The method of any one of claims 1-46, wherein the Compound 1 salt is Compound 1 acetate.
